(19) United States
(12) Patent Application Publication
(10) Pub. No.: US 2026/0027564 A1
(43) Pub. Date: Jan. 29, 2026

(54) SYSTEMS AND METHODS TO FILTER CRUDE SAMPLES FOR NUCLEIC ACID AMPLIFICATION

(71) Applicant: Becton, Dickinson and Company, Franklin Lakes, NJ (US)

(72) Inventors: Tanya Ferguson, San Diego, CA (US); Patrick Truitt, New Freedom, PA (US); Alyssa Shedlosky, Reisterstown, MD (US); Kelsey Ann Graves, Baltimore, MD (US)

(21) Appl. No.: 19/344,319

(22) Filed: Sep. 29, 2025

Related U.S. Application Data
(63) Continuation of application No. PCT/US2024/023558, filed on Apr. 8, 2024.
(60) Provisional application No. 63/495,271, filed on Apr. 10, 2023.
(60) Provisional application No. 63/570,750, filed on Mar. 27, 2024.

(51) Int. Cl. B01L 3/00; C12Q 1/6806; C12Q 1/6844

(52) U.S. Cl. CPC B01L 3/5082; C12Q 1/6806; C12Q 1/6844; B01L 2200/0689; B01L 2300/042; B01L 2300/0681; B01L 2300/0858; B01L 2300/123

(57) ABSTRACT

A filter tube assembly is provided. In one aspect, the filter tube assembly includes a filter, a dispense cap, and a tube body. The filter tube assembly can be used to filter buffer fluid containing a sample in preparation for use in a molecular assay. The filter tube assembly can dispense a fluid volume for a molecular assay in no more than two compressions of the tube body of the filter tube assembly.

### CROSS REFERENCE TO RELATED APPLICATIONS

This application is a continuation of PCT International Application No. PCT/US2024/023558, filed Apr. 8, 2024, which claims the benefit of U.S. Provisional Application No. 63/495,271, filed Apr. 10, 2023, and U.S. Provisional Application No. 63/570,750, filed Mar. 27, 2024, which are hereby incorporated by reference in their entireties.

### BACKGROUND

### Technological Field

The systems and methods disclosed herein are directed to biological sample collection, processing, and testing. More particularly, the systems, devices and methods disclosed herein are directed to a test kit for preparing a sample for use in a molecular assay.

### Description of the Related Technology

Molecular assays can be used in medical diagnosis. For example, molecular assays may be used to test for presence of markers indicative of a particular illness and/or condition.

Nucleic acid amplification is an exemplary molecular assay. The amplification of nucleic acids is important in many fields, including medical, biomedical, environmental, veterinary and food safety testing. Example methods of nucleic acid amplification include polymerase chain reaction (PCR) amplification, isothermal amplification, and/or archaeal polymerase amplification (APA).

Nucleic acid amplification can generate a large number of copies of a target genetic sequence in a test solution. Specific markers can be designed to link to the target sequences as part of a test assay. Once bound, the markers can provide a detectable signal, for example an optical signal, from the test solution. Changes in an optical signal can include changes in the color, opacity, bioluminescence, and/or fluorescence of the test solution. In the case of a fluorescence marker beacon, each marker molecule may be configured with a florescence quencher in close proximity to a fluorescence atom or arrangement of atoms. This marker molecule can be configured such that when selectively bound to a target nucleic acid sequence, the quencher and fluorophore are separated and a fluorescence signal can then be detected by the action of the fluorophore. In this arrangement, the florescence intensity of the target solution is indicative of the relative amount of target genetic material in the test solution. This signal can then be used to form the basis of a diagnostic test to determine the presence or absence and the relative quantity of the target material, or analyte of interest, in the sample under test.

### SUMMARY

The devices, systems, and methods of the present disclosure each have several innovative aspects, no single one of which is solely responsible for its desirable attributes. Without limiting the scope of the present disclosure, its more prominent features will now be discussed briefly.

In one aspect, a filter tube assembly for preparing a sample-containing fluid for use with a molecular assay is provided. The filter tube assembly can include a dispense cap including a flow path and an aperture; a filter positioned within the dispense cap; and a tube body including an open end and a closed end. The tube body is configured to hold a sample-containing fluid, and the dispense cap is configured to secure to the open end of the tube body. At least a portion of the tube body includes a flexible material is configured to be compressed to force one or more continuous streams of the sample-containing fluid through the filter, the flow path, and the aperture when the dispense cap is secured to the open end of the tube body and the flexible material is compressed. The dispense cap can be configured to propel about 2.5 mL to about 3.0 mL of filtered sample-containing fluid through the aperture during no more than two compressions of the flexible material. The filtered sample-containing fluid can be propelled through the aperture in continuous streams.

The filter tube assembly can be configured to propel a continuous stream of at least about 1 mL of filtered sample-containing fluid through the aperture upon a single compression of the tube. The flow path can include a transition zone positioned proximate the filter, the transition zone configured to reduce back pressure on the filter upon compression of the flexible material. A diameter of the filter can be from about 1.2 to about 1.6 times larger than a diameter of the transition zone. The diameter of the transition zone can be between about 8 mm to about 12 mm. The filter tube assembly can include a retaining ring configured to secure the filter to the dispense cap. The retaining ring can include a groove configured to matingly engage with a ridge of the interior surface of the dispense cap. In some other embodiments, the filter is a cup filter comprising walls configured to secure the filter to the dispense cap. The hardness of a material of the dispense cap can be greater than the hardness of a material of the tube body. The Young's modulus of a material of the dispense cap can be at least twice as large as the Young's modulus of a material of the tube body. The Young's modulus of a material of the dispense cap can be at least about 800 MPa. The Young's modulus of a material of the tube body is from about 200 to about 300 MPa. The dispense cap can include a high-density polyethylene (HDPE) or a polypropylene. The tube body material can include a low-density polyethylene (LDPE) or a linear low-density polyethylene (LLDPE). The dispense cap can include a tether ring configured to engage the exterior of the tube body. The filter tube assembly can include a travel cap configured to secure to the open end of the tube body. The travel cap can include a collar configured create a plug seal thereby inhibiting leakage of a fluid from an interior volume of the tube body when the travel cap is secured to the open end of the tube body. The travel cap can include a threaded portion configured to engage a threaded portion of the tube body. The dispense cap can comprise a threaded portion configured to engage a threaded portion of the tube body. The tube body can be at least partially transparent to visible light. A thickness of at least a portion of walls of the tube body can be between about 0.20 mm and about 1 mm. The filter can include a plurality of openings having diameter between about 1 μm and about 250 μm. The filter can include a polyethylene, a glass fiber, a polypropylene, or a polytetrafluoroethylene. The tube body can have an interior volume between about 3 mL and about 5 mL. The tube body can be configured to hold from about 2.5 mL to about 3.5 mL of sample-containing fluid. The tube body can be configured to include at least 2 mL of headspace volume when the sample-containing fluid is present in the tube body. A diameter of the aperture can be between about 2.8 mm and about 3.2 mm. The filter can be configured to filter molecules entering through a first surface of the filter and exiting through an opposing second surface of the filter. The surface area of the first surface of the filter can be from about 100 mm² to about 200 mm². A ratio a:v of the surface area of the first surface of the filter to the internal volume of the tube body can be between about 20 m⁻¹ to about 40 m⁻¹. The tube body can include a snap fit ridge and a flange. The snap fit ridge can be configured to matingly engage with an indentation of the dispense cap. The flange can be configured to contact a proximal end of the dispense cap. The force required to snap fit the dispense cap to the tube body can be from about 20 N to 100 N. The force required to remove the dispense cap from the tube body after being snap fit can be at least about 30 N. The tube body and dispense cap can be cast as a single piece of plastic. In embodiments where the tub body and dispense cap are cast as a single piece of plastic, the filter tube assembly may include at least one tether connecting the tube body to the dispense cap. In embodiments where the tub body and dispense cap are cast as a single piece of plastic, the plastic of the tube body and dispense cap may have a Young's modulus ranging between 200 and 700 MPa. A seal can cover the open end of the tube body. The tube body can include a fill line indicative of a minimum amount of volume for the molecular assay. The filter tube assembly can include a buffer. The buffer can be contained within an interior volume of the tube body. The filter can be configured to prepare the sample-containing fluid for use in a gonorrhea assay, a chlamydia assay, or a trichomoniasis assay. The molecular assay can be a point-of-care molecular assay. The dispense cap can include a collar, the tube body can include a neck region that can engage the collar of the dispense cap to form a fluid-tight seal (for example, a plug seal). The collar and neck region can engage via an interference fit. A thickness of the wall at a proximal end of the dispense cap collar may be less than a thickness of the wall at a distal end of the dispense cap collar. An outer diameter of the wall at a proximal end of the dispense cap collar may be less than an outer diameter of the wall at a distal end of the dispense cap collar. The dispense cap collar may include a first ridge, the neck region of the tube body may include a second ridge, and the first ridge and the second ridge can slidingly engage. The tube body can include a base portion positioned proximate the closed end of the tube body, the base region having a diameter that is less than a diameter of the collar. The dispense cap can include one or more retention bumps configured to retain the filter within the dispense cap.

In another aspect, a method of preparing a sample for a molecular assay is provided. The method can include securing a dispense cap to an open end of a tube body, the dispense cap including a filter. The method can include compressing the tube body no more than two times, thereby propelling a sample-containing buffer from an interior volume of the tube body through the filter and out the dispense cap in continuous streams, while retaining in the filter at least some inhibitors of a molecular assay from the sample-containing buffer. The method can include performing the molecular assay using the filtered sample-containing buffer.

Compressing the tube body can propel at least from about 2.5 mL to about 3.0 mL of filtered sample-containing buffer from the dispense cap. Securing the dispense cap to the tube body can include securing the dispense cap to the tube body via a snap-fit connection. The molecular assay can include amplification of nucleic acids in the filtered sample-containing buffer. The method can include introducing the sample to a buffer to form the sample-containing buffer, the buffer held within the interior volume of the tube body. Compressing the tube body can include one, two, or three compressions of the tube body. The molecular assay can be an amplification assay. The method can include removing a travel cap from the open end of the tube body before securing the dispense cap to the open end of the tube body. The method can include removing and/or breaking a seal covering the open end of the tube body. The dispense cap can include a flow path and an aperture. The flow path can include a transition zone. A diameter of the filter can be about 1.2 to about 1.6 times larger than a diameter of the transition zone of the flow path. The diameter of the filter can range from about 12 mm to about 16 mm. The diameter of the transition zone can range from about 8 mm to about 12 mm. Compressing the tube body can include filtering molecules from the sample-containing buffer that enters a first surface of the filter and exits through a second surface of the filter, the surface area of the filter being between about 100 mm² and about 200 mm². A ratio av of the surface area of the first surface of the filter to the internal volume of the tube body can be between about 20 m⁻¹ to about 40 m⁻¹.

In yet another aspect, a filter tube assembly for preparing a sample-containing fluid for use with a molecular assay is provided. The filter tube assembly includes a dispense cap including a flow path and an aperture. The flow path can include a transition zone. The filter tube assembly can also include a filter positioned within the dispense cap, a diameter of the filter about 1.2 to about 1.6 times larger than a diameter of the transition zone of the flow path. The filter tube assembly includes a tube body including an open end and a closed end, the tube body configured to hold a sample-containing fluid. The dispense cap can be configured to secure to the open end of the tube body. At least a portion of the tube body can include a flexible material configured to be compressed to force at least a portion of the sample-containing fluid through the filter, the flow path, and the aperture when the dispense cap is secured to the open end of the tube body and the flexible material is compressed. The dispense cap can be configured to propel continuous streams of filtered sample-containing fluid through the aperture. The diameter of the filter can range from about 12 mm to about 16 mm. The diameter of the transition zone can range from about 8 mm to about 12 mm.

In still another aspect, a filter tube assembly for preparing a sample-containing fluid for use with a molecular assay is provided. The filter tube assembly includes a dispense cap including a flow path and an aperture. The filter tube assembly can include a filter positioned within the dispense cap, the filter configured to filter molecules from a sample-containing fluid entering through a first surface of the filter and exiting through a second surface of the filter. The surface area of the filter can be between about 100 mm² and about 200 mm². The filter tube assembly can include a tube body including an open end and a closed end, the tube body configured to hold the sample-containing fluid in an internal volume. The dispense cap can be configured to secure to the open end of the tube body. At least a portion of the tube body can include a flexible material configured to be compressed to force at least a portion of the sample-containing fluid through the filter, the flow path, and the aperture when the dispense cap is secured to the open end of the tube body and the flexible material is compressed. The dispense cap can be configured to propel continuous streams of filtered sample-containing fluid through the aperture. A ratio a:v of the surface area of the first surface of the filter to the internal volume of the tube body can be between about 20 m⁻¹ to about 40 m⁻¹.

Embodiments provided herein include the following numbered Embodiments:
1. A filter tube assembly for preparing a sample-containing fluid for use with a molecular assay, the assembly comprising:
   a dispense cap comprising a flow path and an aperture,
   a filter positioned within the dispense cap; and
   a tube body comprising an open end and a closed end, the tube body configured to hold a sample-containing fluid, the dispense cap configured to secure to the open end of the tube body, at least a portion of the tube body comprising a flexible material configured to be compressed to force at least a portion of the sample-containing fluid through the filter, the flow path, and the aperture when the dispense cap is secured to the open end of the tube body and the flexible material is compressed, the dispense cap configured to propel about 2.5 mL to about 3.0 mL of filtered sample-containing fluid in continuous streams through the aperture during no more than two compressions of the flexible material.
2. The filter tube assembly of embodiment 1, wherein the filter tube assembly is configured to propel a continuous stream of at least about 1 mL of filtered sample-containing fluid through the aperture upon a single compression of the tube.
3. The filter tube assembly of embodiment 1 or 2, wherein the flow path comprises a transition zone positioned proximate the filter, the transition zone configured to reduce back pressure on the filter upon compression of the flexible material.
4. The filter tube assembly of embodiment 3, wherein a diameter of the filter is from about 1.2 to about 1.6 times larger than a diameter of the transition zone.
5. The filter tube assembly of embodiment 4, wherein the diameter of the transition zone is between about 8 mm to about 12 mm.
6. The filter tube assembly of any of embodiments 1 to 5, comprising a retaining ring configured to secure the filter to the dispense cap.
7. The filter tube assembly of embodiment 6, wherein the retaining ring comprises a groove configured to matingly engage with a ridge of the interior surface of the dispense cap.
8. The filter tube assembly of any of embodiments 1 to 5, wherein the filter is a cup filter comprising walls configured to secure the filter to the dispense cap.
9. The filter tube assembly of any of embodiments 1 to 8, wherein the hardness of a material of the dispense cap is greater than the hardness of a material of the tube body.
10. The filter tube assembly of embodiment 9, wherein the Young's modulus of a material of the dispense cap is at least twice as large as the Young's modulus of a material of the tube body.
11. The filter tube assembly of any of embodiments 1 to 10, wherein the Young's modulus of a material of the dispense cap is at least about 800 MPa.
12. The filter tube assembly of any of embodiments 1 to 11, wherein the Young's modulus of a material of the tube body is from about 200 to about 300 MPa.
13. The filter tube assembly of any of embodiments 1 to 12, wherein the dispense cap comprises a high-density polyethylene (HDPE) or a polypropylene.
14. The filter tube assembly of any of embodiments 1 to 13, wherein the tube body material comprises a linear low-density polyethylene (LLDPE).
15. The filter tube assembly of any of embodiments 1 to 14, the dispense cap further comprising a tether ring configured to engage the exterior of the tube body.
16. The filter tube assembly of any of embodiments 1 to 15, further comprising a travel cap configured to secure to the open end of the tube body, the travel cap comprising a collar configured to inhibit leakage of a fluid from an interior volume of the tube body when the travel cap is secured to the open end of the tube body.
17. The filter tube assembly of embodiment 16, wherein the travel cap comprises a threaded portion configured to engage a threaded portion of the tube body.
18. The filter tube assembly of any of embodiments 1 to 17, wherein the dispense cap comprises a threaded portion configured to engage a threaded portion of the tube body.
19. The filter tube assembly of any of embodiments 1 to 18, wherein the tube body is at least partially transparent to visible light.
20. The filter tube assembly of any of embodiments 1 to 19, wherein a thickness of at least a portion of walls of the tube body is between about 0.2 mm and about 1.0 mm.
21. The filter tube assembly of any of embodiments 1 to 20, wherein the filter comprises a plurality of openings having diameter between about 1 μm and about 250 μm.
22. The filter tube assembly of any of embodiments 1 to 21, wherein the filter comprises a polyethylene, a glass fiber, a polypropylene, or a polytetrafluoroethylene.
23. The filter tube assembly of any of embodiments 1 to 22, wherein the tube body has an interior volume between about 3 mL and about 5 mL.
24. The filter tube assembly of any of embodiments 1 to 23, wherein the tube body is configured to hold from about 2.5 mL to about 3.5 mL of sample-containing fluid.
25. The filter tube assembly of any of embodiments 1 to 24, wherein the tube body is configured to include at least 2 mL of headspace volume when the sample-containing fluid is present in the tube body.
26. The filter tube assembly of any of embodiments 1 to 25, wherein the aperture comprises a diameter, and wherein the diameter is between about 2.8 mm and about 3.2 mm.
27. The filter tube assembly of any of embodiments 1 to 26, wherein the filter is configured to filter molecules entering through a first surface of the filter and exiting through an opposing second surface of the filter, and wherein the surface area of the first surface of the filter is from about 100 mm² to about 200 mm².
28. The filter tube assembly of embodiment 27, wherein a ratio a:v of the surface area of the first surface of the filter to the internal volume of the tube body is between about 20 m⁻¹ to about 40 m⁻¹.
29. The filter tube assembly of any of embodiments 1 to 28, wherein the tube body further comprises a snap fit ridge and a flange, the snap fit ridge configured to matingly engage with an indentation of the dispense cap, the flange configured to contact a proximal end of the dispense cap.
30. The filter tube assembly of any of embodiments 1 to 29, wherein the force required to attach the dispense cap to the tube body is from about 20 N to 100 N.
31. The filter tube assembly of any of embodiments 1 to 30, wherein the force required to remove the dispense cap from the tube body after being snap fit is at least about 30 N.
32. The filter tube assembly of any of embodiments 1 to 8 or 16 to 31, wherein the tube body and dispense cap are cast as a single piece of plastic, and wherein the filter tube assembly comprises at least one tether connecting the tube body to the dispense cap.
33. The filter tube assembly of embodiment 32, wherein the plastic of the tube body and dispense cap has a Young's modulus between 200 and 700 MPa.
34. The filter tube assembly of embodiment 32 or 33, comprising a seal covering the open end of the tube body.
35. The filter tube assembly of any of embodiments 1 to 34, wherein the tube body comprises a fill line indicative of a minimum amount of volume for the molecular assay.
36. The filter tube assembly of any of embodiments 1 to 35, further comprising a buffer contained within an interior volume of the tube body.
37. The filter tube assembly of any of embodiments 1 to 36, wherein the filter is configured to prepare the sample-containing fluid for use in a gonorrhea assay, a chlamydia assay, or a trichomoniasis assay.
38. The filter tube assembly of any of embodiments 1 to 37, wherein the molecular assay is a point-of-care molecular assay.
39. The filter tube assembly of any of embodiments 1 to 38, wherein the dispense cap further comprises a collar, and the tube body comprises a neck region configured to engage the collar of the dispense cap to form a fluid-tight seal, wherein the collar and neck region are configured to engage via an interference fit.
40. The filter tube assembly of embodiment 39, wherein a thickness of the wall at a proximal end of the dispense cap collar is less than a thickness of the wall at a distal end of the dispense cap collar.
41. The filter tube assembly of embodiment 39 or 40, wherein an outer diameter of the wall at a proximal end of the dispense cap collar is less than an outer diameter of the wall at a distal end of the dispense cap collar.
42. The filter tube assembly of any of embodiments 39 to 41, wherein the dispense cap collar comprises a first ridge and the neck region of the tube body comprises a second ridge, the first ridge and the second ridge configured to slidingly engage.
43. The filter tube assembly of any of embodiments 39 to 42, wherein the tube body further comprises a base portion positioned proximate the closed end of the tube body, the base portion comprising a diameter that is less than a diameter of the neck region.
44. The filter tube assembly of any of embodiments 1 to 43, wherein the dispense cap comprises one or more retention bumps configured to retain the filter within the dispense cap.
45. A method of preparing a sample for a molecular assay, comprising:
   securing a dispense cap to an open end of a tube body, the dispense cap comprising a filter;
   compressing the tube body no more than two times, thereby propelling a sample-containing buffer from an interior volume of the tube body through the filter and out the dispense cap in continuous streams, while retaining in the filter at least some inhibitors of a molecular assay from the sample-containing buffer; and
   performing the molecular assay using the filtered sample-containing buffer.
46. The method of embodiment 45, wherein compressing the tube body propels at least from about 2.5 mL to about 3.0 mL of filtered sample-containing buffer from the dispense cap.
47. The method of embodiment 45 or 46, wherein securing the dispense cap to the tube body comprises securing the dispense cap to the tube body via a snap-fit connection.
48. The method of any of embodiments 45 to 47, wherein the molecular assay comprises amplification of nucleic acids in the filtered sample-containing buffer.
49. The method of any of embodiments 45 to 48, further comprising introducing the sample to a buffer to form the sample-containing buffer, the buffer held within the interior volume of the tube body.
50. The method of any of embodiments 45 to 49, wherein the molecular assay is an amplification assay.
51. The method of any of embodiments 45 to 50, further comprising removing a travel cap from the open end of the tube body before securing the dispense cap to the open end of the tube body.
52. The method of any of embodiments 45 to 51, further comprising removing a seal covering the open end of the tube body.
53. The method of any of embodiments 45 to 52, wherein the dispense cap comprises a flow path and an aperture, the flow path comprises a transition zone, and a diameter of the filter is about 1.2 to about 1.6 times larger than a diameter of the transition zone of the flow path.
54. The method of any of embodiments 45 to 53, wherein the diameter of the filter ranges from about 12 mm to about 16 mm and the diameter of the transition zone ranges from about 8 mm to about 12 mm.
55. The method of any of embodiments 45 to 54, wherein compressing the tube body comprises filtering molecules from the sample-containing buffer that enters a first surface of the filter and exits through a second surface of the filter, the surface area of the filter being between about 100 mm² and about 200 mm².
56. The method of any of embodiments 45 to 55, wherein a ratio a:v of the surface area of the first surface of the filter to the internal volume of the tube body is between about 20 m⁻¹ to about 40 m⁻¹.
57. A filter tube assembly for preparing a sample-containing fluid for use with a molecular assay, the assembly comprising:
   a dispense cap comprising a flow path and an aperture, the flow path comprising a transition zone,
   a filter positioned within the dispense cap, a diameter of the filter about 1.2 to about 1.6 times larger than a diameter of the transition zone of the flow path; and
   a tube body comprising an open end and a closed end, the tube body configured to hold a sample-containing fluid, the dispense cap configured to secure to the open end of the tube body, at least a portion of the tube body comprising a flexible material configured to be compressed to force at least a portion of the sample-containing fluid through the filter, the flow path, and the aperture when the dispense cap is secured to the open end of the tube body and the flexible material is compressed, the dispense cap configured to propel continuous streams of filtered sample-containing fluid through the aperture.
58. The filter tube assembly of embodiment 57, wherein the diameter of the filter ranges from about 12 mm to about 16 mm and the diameter of the transition zone ranges from about 8 mm to about 12 mm.
59. A filter tube assembly for preparing a sample-containing fluid for use with a molecular assay, the assembly comprising:
   a dispense cap comprising a flow path and an aperture,
   a filter positioned within the dispense cap, the filter configured to filter molecules from a sample-containing fluid entering through a first surface of the filter and exiting through a second surface of the filter, the surface area of the filter being between about 100 mm² and about 200 mm²; and
   a tube body comprising an open end and a closed end, the tube body configured to hold the sample-containing fluid in an internal volume, the dispense cap configured to secure to the open end of the tube body, at least a portion of the tube body comprising a flexible material configured to be compressed to force at least a portion of the sample-containing fluid through the filter, the flow path, and the aperture when the dispense cap is secured to the open end of the tube body and the flexible material is compressed, the dispense cap configured to propel continuous streams of filtered sample-containing fluid through the aperture.
60. The filter tube assembly of embodiment 59, wherein a ratio a:v of the surface area of the first surface of the filter to the internal volume of the tube body is between about 20 m⁻¹ to about 40 m⁻¹.

### BRIEF DESCRIPTION OF THE DRAWINGS

The above-mentioned aspects, as well as other features, aspects, and advantages of embodiments of the present disclosure will now be described in connection with various implementations, with reference to the accompanying drawings. The illustrated implementations are merely examples and are not intended to be limiting. Throughout the drawings, similar symbols typically identify similar components, unless context dictates otherwise.

FIGS. 1A-1B illustrate views of an example filter tube assembly that can be used for preparing a sample for a molecular assay according to an embodiment of the present disclosure, where the tube body and dispense cap are separate parts.

FIGS. 2A-2F illustrate views of the dispense cap included in the filter tube assembly of FIGS. 1A-1B.

FIGS. 3A-3C illustrate views of a filter included in the filter tube assembly of FIGS. 1A-1B.

FIGS. 4A-4C illustrate views of a retaining ring included in the filter tube assembly of FIGS. 1A-1B.

FIGS. 5A-5D illustrate views of a tube body included in the filter tube assembly of FIGS. 1A-1B.

FIG. 6 illustrates a cross-sectional view of the filter tube assembly of FIGS. 1A-1B.

FIG. 7 illustrates a cross-sectional view of the filter and retaining ring positioned within the dispense cap of the filter tube assembly of FIGS. 1A-1B.

FIGS. 8A-8C illustrate example steps of attaching the retaining ring to the tube body and closing the open end of the tube body with the dispense cap according to an embodiment of the present disclosure.

FIGS. 9A-9D illustrate views of an example travel cap with the tube body according to an embodiment of the present disclosure.

FIGS. 10A-10D illustrate views of another example filter tube assembly that can be used for preparing a sample for a molecular assay according to an embodiment of the present disclosure, where the tube body and the dispense cap are a single part.

FIGS. 11A-11D illustrate views of an example filter tube assembly that can be used for preparing a sample for a molecular assay according to an embodiment of the present disclosure, where the tube body and the dispense cap are a single part.

FIG. 12A-12D illustrate views of the dispense cap included in the filter tube assembly of FIGS. 11A-11D.

FIGS. 13A-13D illustrate views of a filter included in the filter tube assembly of FIGS. 11A-11D.

FIGS. 14A-14D illustrate views of a tube body included in the filter tube assembly of FIGS. 11A-11D.

FIGS. 15A-15B illustrate views of a seal that can be used with the filter tube assembly of FIGS. 11A-11D.

FIGS. 16A-17 illustrate cross-sectional views of the filter tube assembly of FIGS. 11A-11D showing interaction of the dispense cap, the tube body, and the filter.

FIGS. 18A-18D illustrate example steps of preparing the filter tube assembly of FIGS. 11A-11D for use.

FIGS. 19A-19D illustrate views of another example filter tube assembly that can be used for preparing a sample for a molecular assay according to an embodiment of the present disclosure, where the tube body and dispense cap are separate parts.

FIGS. 20A-20E illustrate views of the dispense cap included in the filter tube assembly of FIGS. 19A-19D.

FIGS. 21A-21D illustrate views of a tube body included in the filter tube assembly of FIGS. 19A-19D.

FIG. 22 illustrates a view of the filter tube assembly of FIGS. 19A-19D with a seal.

FIGS. 23A-24 illustrate cross-sectional views of the filter tube assembly of FIGS. 19A-19D showing interaction of the dispense cap, the tube body, and the filter.

FIG. 25A-25D illustrate example steps of preparing the filter tube assembly of FIGS. 19A-19D for use.

FIG. 26 is an example flow diagram of a method of using a filter tube assembly according to the present disclosure.

FIG. 27 plots optical density at a wavelength of 600 nm of sample-containing fluid after filtration using filter tube assemblies according to the present disclosure.

FIG. 28 is a box plot of the concentration of human genomic DNA of sample-containing fluid after filtration using filter tube assemblies according to the present disclosure.

FIGS. 29A-29C plot fluorescence over time for molecular assays for a urine sample filtered using filter tube assemblies according to the present disclosure and for an unfiltered urine sample.

FIG. 30 plots average cycle threshold (Ct) of PCR assays for Trichomonas vaginalis and Neisseria gonorrhoeae using unfiltered samples and samples filtered using filter tube assemblies according to the present disclosure.

### DETAILED DESCRIPTION

Embodiments of the present disclosure provide devices, systems, and methods that can prepare a solution, such as a fluid sample, for use in a molecular assay. Such molecular assays can be used for detection of, for example, genomic material. The genomic material may originate from a specimen, for example a bacteria, a virus, a yeast, and/or a parasite. The fluid sample can include a test sample in a buffer solution. In some cases, the fluid sample is amplification-ready after transfer from an interior volume of the device, through a filter of the device, and propelled in one or more streams out of the device (for example, one or more continuous streams of filtered fluid sample out of the device). Filtering the fluid sample may prepare the sample for inclusion in a molecular assay, for example by removing molecules that may interfere with the molecular assay. For example, a portion of the fluid sample can be transferred from an interior volume of the device to a test container where a molecular assay, for example an amplification assay, may take place. Throughout this disclosure, example systems, kits, and methods will be described with reference to collection, filtering, testing, and detection of nucleic acids, but it will be understood that the present technology can be used to collect, test, and detect any particle, molecule, or analyte of interest.

Embodiments of devices, systems, and methods according to the present disclosure can advantageously prepare complex, crude matrices for detection of nucleic acids of interest in the point-of-care setting, allowing removal of assay inhibitors and clarification of a specimen through filtration. In particular, embodiments of devices, system, and methods described herein can clarify a complex, crude specimen, such as vaginal matrix and urine, throat, and nasal swab matrix, using a non-instrumented approach that is compatible with CLIA waiver in the point-of-care setting. Thus, devices, system, and methods of the present disclosure advantageously omit instrumentation and additional steps required to use such instrumentation, such as centrifugation and solid-phase purification. Advantageously, known inhibitors of nucleic acid amplification are removed from the crude matrix, with little to no loss of analyte of interest from the specimen. Accordingly, embodiments of the present disclosure can detect target analytes, such as bacteria, virus, yeast, and parasites, with increased sensitivity. In addition, embodiments of devices, system, and methods according to the present disclosure perform with little to no clogging of the filtration element, an event that would result in a failed test event.

As an illustrative example, a collection kit including the filter tube assembly disclosed herein can include a swab, the filter tube assembly, and a test device. The swab can be constructed from a material having desired abrasiveness, pickup efficiency, and shedding efficiency for detecting analytes of interest such as nucleic acids. The swab can be provided on a handle. The user can use the swab and handle to collect a sample, for example a vaginal swab, a throat swab, or a nasal swab. After swabbing, the user can place the swab into a tube body of the filter tube assembly. The handle may include a break point so that the user can break off the swab within the tube body by angling the handle against the interior surface of the tube body, without physically contacting the swab against a surface that could potentially contaminate the swab. In some examples, the sample can be a fluid sample, for example an oral fluid sample or a urine fluid sample from a patient.

A liquid, for example an extraction buffer, can be provided within the tube body of the filter tube assembly. In examples using a swab, the swab can absorb the extraction buffer when it is placed in the tube body of the filter tube assembly. The user can close a dispense cap of the filter tube assembly over the tube body of the filter tube assembly. The user can compress the tube body, thereby causing the swab to express absorbed extraction buffer and any collected analyte of interest, such as nucleic acids. In some examples where the user does not break off the swab within the tube body, the user can add any collected analyte of interest on the swab to the extraction buffer by mixing the swab in the extraction buffer within the tube body, then removing the swab from the tube body. In some examples, for instance where a fluid sample is collected, the fluid sample can be added directly to the extraction buffer within the tube body. After the sample is added to the extraction buffer and the dispense cap of the filter tube assembly is closed over the tube body, the user can compress the tube body to propel the sample-containing fluid through the filter of the filter tube assembly, through a fluid path of the dispense cap, and to the test device. Filtered sample-containing fluid produced by a device or system of the present disclosure can be used in a molecular assay, for example but not limited to a gonorrhea assay, a chlamydia assay, or a trichomoniasis assay. The molecular assay may be a point-of-care assay. For instance, the molecular assay may be performed in a clinician's and/or healthcare provider's office, or the like.

Various embodiments will be described below in conjunction with the drawings for purposes of illustration. It should be appreciated that many other implementations of the disclosed concepts are possible, and various advantages can be achieved with the disclosed implementations.

A. Filter Tube Assembly with a Disc Filter and Separate Tube Body and Dispense Cap

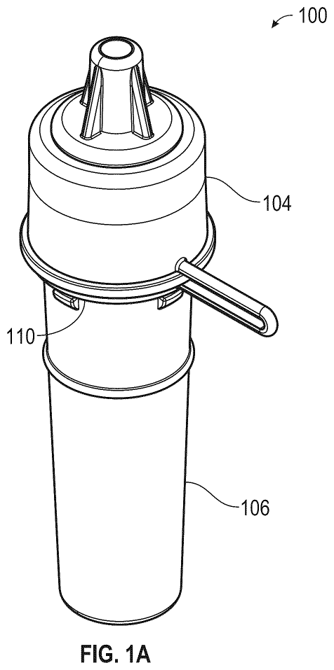

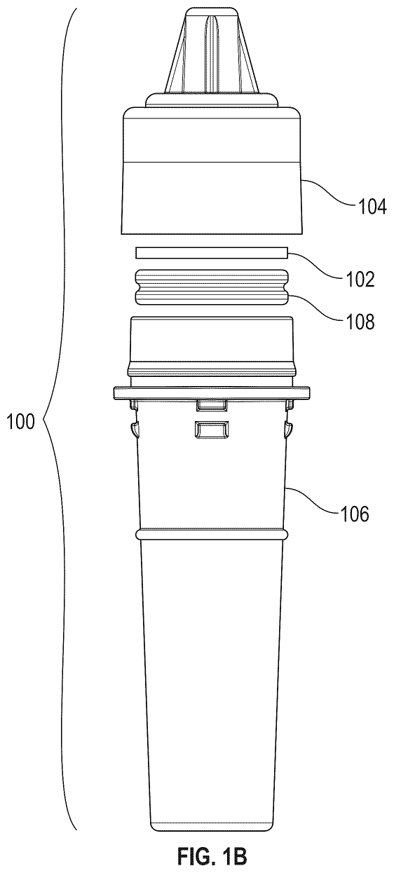

FIGS. 1A-1B illustrate an example filter tube assembly that can be used for preparing a sample for a molecular assay according to an embodiment of the present disclosure. FIG. 1A illustrates an example filter tube assembly 100 in a side view. FIG. 1B illustrates the example filter tube assembly 100 of FIG. 1A in an exploded side view. The filter tube assembly 100 can include a filter 102 (for example, a disc filter), a dispense cap 104, and a tube body 106. In some embodiments, the filter tube assembly 100 can include a retaining ring 108 and a tether ring 110. The filter tube assembly 100 may be used to prepare a sample-containing fluid for use in a molecular assay. The tube body 106 may be capable of holding a fluid, for example a buffer or a sample-containing fluid. When open, the tube body 106 may be capable of receiving a sample, for example a swab on which a sample has been collected. When the dispense cap 104 is attached to the tube body 106 and the tube body 106 is compressed, the filter tube assembly 100 can propel the sample-containing fluid from an interior volume of the tube body 106 through the filter 102 and out from the dispense cap 104.

In some examples, the filter tube assembly 100 is capable of propelling the filtered sample-containing fluid out the dispense cap 104 in one or more streams (for example, at a significantly higher dispense rate than a dropper). In some embodiments of the filter tube assembly according to the present disclosure, the dispense cap is configured to propel a continuous stream of filtered sample-containing fluid through the aperture during each compression of the tube body. Advantageously, embodiments of the filter tube assembly can thus deliver high volumes of filtered sample-containing fluid out of the aperture rapidly, precisely, and with minimal user interaction, thereby minimizing the risk that the filtered sample-containing fluid will become exposed to environmental or user-derived contaminants, degraded, or compromised before the filtered sample-containing fluid is delivered to a test device where a molecular assay is performed. In one non-limiting embodiment described below, a filter tube assembly according to the present disclosure is configured to propel about 2.5 to about 3.0 mL of filtered sample-containing fluid in continuous streams through the aperture during one, two, three, or four compressions of the tube body. In one example, a total volume of about 2.5 to about 3.0 mL of filtered sample-containing fluid is propelled through the aperture during no more than two compressions of the tube body. Each compression may result in a continuous stream of a plurality of continuous streams of fluid being propelled through the aperture. Alternatively, the compressions of the tube body may result in a single continuous stream of fluid being propelled through the aperture.

Once filtered using the filter tube assembly 100, the sample-containing fluid may be suitable for use in a molecular assay. For example, the filter tube assembly 100 may be capable of filtering out certain molecules and/or particles capable of interfering with the molecular assay. Example inhibitors that can be removed by embodiments of filter tube assemblies described herein include proteins (blood and non-blood based), carbohydrates (for example mucin), immunoglobulins, cells and cellular debris, host microbiome, human genomic DNA (hugDNA), and salts. Advantageously, embodiments of filter tube assemblies according to the present disclosure can remove these and other inhibitors, thereby significantly improving assay sensitivity, decreasing time for DNA and RNA amplification, and in some cases preventing a failed test resulting from excessive inhibitors in a crude matrix.

Molecular assays may require a relatively high volume of sample-containing fluid in comparison to other assay types (for example, immunoassays). Thus, it may be desirable that the filter tube assembly 100 according to embodiments of the present disclosure can quickly and/or easily dispense a total volume as provided herein. The filter tube assembly 100 can propel the filtered sample-containing fluid out of the dispense cap, such that filtration is relatively quick (for example, within seconds of or immediately in response to compression of the tube body 106). In some examples, the filter tube assembly 100 is capable of releasing the total volume of filtered sample-containing fluid in no more than two compressions of the tube body 106. The compressions can be provided by a user compressing the tube body 106. In some other examples, the filter tube assembly 100 is capable of releasing the total volume of filtered sample-containing fluid in no more than one, two, three, or four compressions of the tube body 106.

In some examples, the filter tube assembly 100 can dispense a total volume of about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 mL of filtered fluid, or any value or range within or bounded by any of these ranges or values, although values outside these values or ranges can be used in some cases. Additionally or alternatively, in some examples the filter tube assembly 100 can dispense a total of about 2 to 4 mL of filtered fluid. Additionally or alternatively, in some examples the filter tube assembly 100 can dispense a total of about 2.5 mL to about 3 mL of filtered fluid.

In some examples, upon a single compression of the tube body 106, the filter tube assembly 100 can dispense a total of about 0.4, 0.5, 0.6, 0.7, 0.8, 0.9, 1.0, 1.1, 1.2, 1.3, 1.4, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mL of filtered fluid, or any value or range within or bounded by any of these ranges or values, although values outside these values or ranges can be used in some cases. Additionally or alternatively, in some examples the filter tube assembly 100 can dispense a total of about 0.5 to 2 mL of filtered fluid per compression of the tube body 106. Additionally or alternatively, in some examples the filter tube assembly 100 can dispense a total of about 0.5 to 1.5 mL of filtered fluid per compression of the tube body 106.

### 1. Dispense Cap

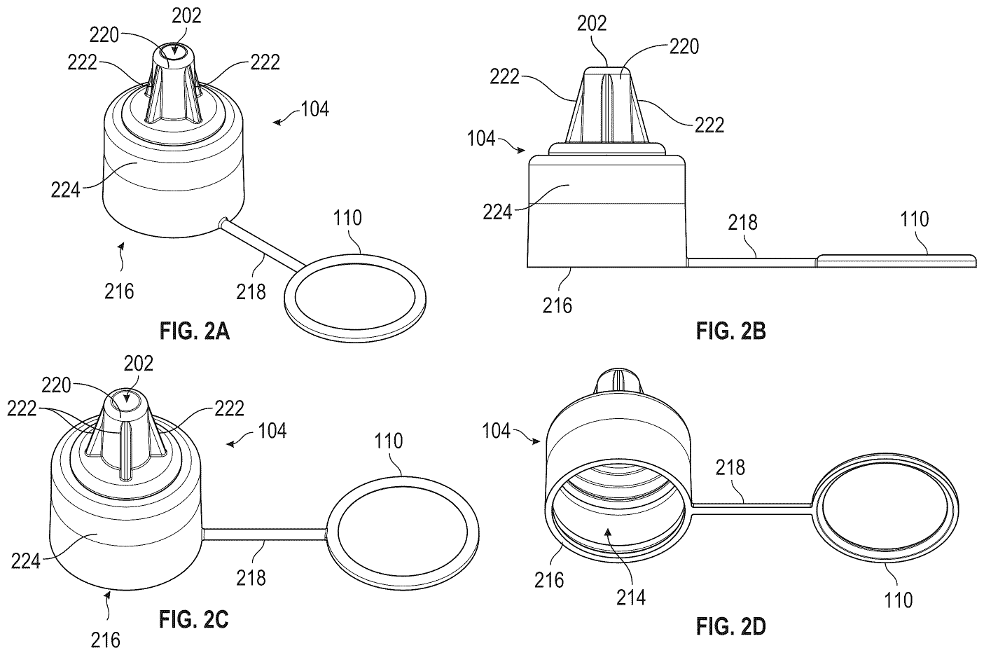

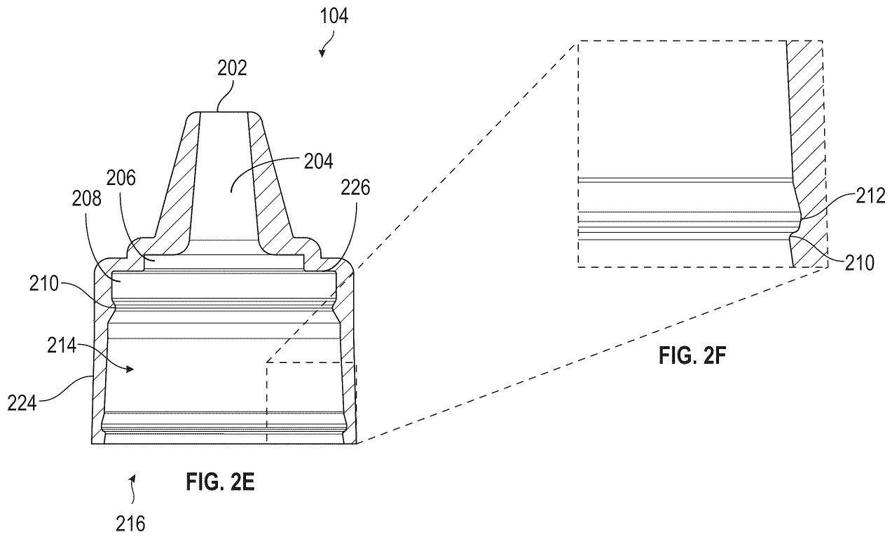

FIGS. 2A-2F illustrate views of the dispense cap 104 included in the filter tube assembly 100 according to an embodiment of the present disclosure. FIG. 2A illustrates an angled view of the dispense cap 104. FIG. 2B illustrates a side view of the dispense cap 104. FIG. 2C illustrates an angled side view of the dispense cap 104. FIG. 2D illustrates an angled bottom view of the dispense cap 104. FIG. 2E illustrates a cross-sectional side view of dispense cap 104. FIG. 2F illustrates a zoomed-in view of the cross-sectional view of FIG. 2E.

The dispense cap 104 can include an aperture 202, a flow path 204, a filter cavity 208, an interior space 214, a proximal end 216, a nozzle 220, and a cap body 224. In some embodiments, the dispense cap 104 can also include a tether ring 110 and an arm 218. In some embodiments, the dispense cap 104 can include buttresses 222. The dispense cap 104 can also include one or more ridges 210 and an indentation 212. The flow path 204 can include a transition zone 206.

The flow path 204 may be a hollow space within the dispense cap 104, for example within the nozzle 220 of the dispense cap 104. The aperture 202 may be a hole at the distal end of the nozzle 220. The flow path 204 may be capable of allowing one or more propelled streams of sample-containing fluid (for example, filtered sample-containing fluid) to flow out the dispense cap 104. In some examples, the diameter of the aperture 202 may be about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mm, or any value or range within or bounded by any of these ranges or values, although values outside these values or ranges can be used in some cases. Additionally or alternatively, in some examples the aperture 202 may have a diameter of about 2 to 4 mm. Additionally or alternatively, in some examples the aperture 202 may have a diameter of about 2.8 to 3.2 mm. The filter cavity 208 may be a space that can accept and hold a filter 102 in accordance with embodiments of the present disclosure. The interior walls of the filter cavity 208 can contact the filter 102 when the filter 102 is positioned within the dispense cap 104. The filter 102 may contact the distal surface 226 of the filter cavity 208, for example when the filter 102 is secured within the filter cavity 208 by the retaining ring 108. The transition zone 206 may be positioned proximate the filter cavity 208.

The transition zone 206 can provide a volume where sample-containing fluid can be propelled out of the filter 102. The transition zone 206 has a diameter larger than the rest of the flow path 204. Thus, because the transition zone 206 does not greatly constrict the diameter of the flow path, the transition zone 206 can thereby decrease back pressure (that is, a pressure within the tube body 106 when, for example, the tube body 106 is compressed) exerted on the filter 102. By decreasing the pressure exerted on the filter 102, the transition zone 206 may reduce a risk of breakage, rupture, and/or slippage of the filter 102 when the tube body 106 is compressed. In some examples, the diameter of the transition zone 206 can be about 4, 5, 6, 7, 8, 9, 10, 11, 12, 13, 14, 15, 16, 17, 18, 19, or 20 mm, or any value or range within or bounded by any of these ranges or values, although values outside these values or ranges can be used in some cases. Additionally or alternatively, in some examples the transition zone 206 may have a diameter of about 8 to 12 mm. Additionally or alternatively, in some examples the transition zone 206 may have a diameter of about 10 mm.

The nozzle 220 may extend away from the cap body 224. The interior surface of the nozzle 220 may define a portion of the flow path 204. In some embodiments, the dispense cap 104 may be supported by the buttresses 222. The buttresses 222 may stabilize the nozzle 220 and/or prevent or inhibit breakage of the nozzle 220.

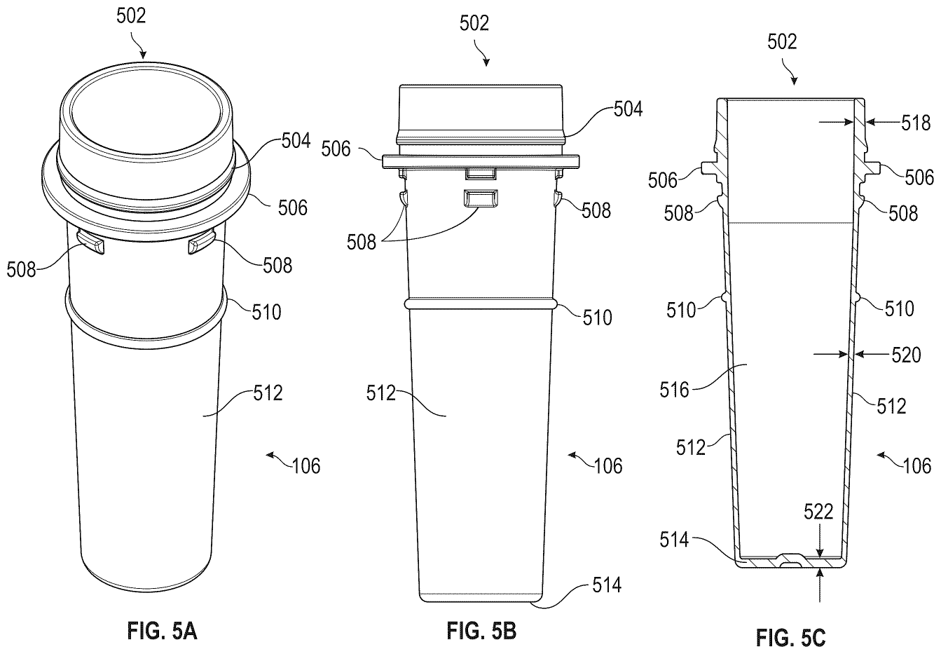

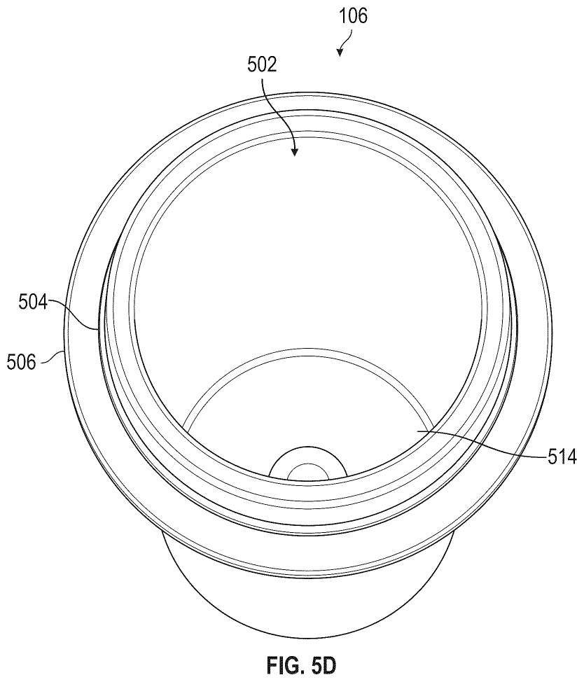

The arm 218 may extend from the cap body 224 to the tether ring 110. The arm 218 may be able to bend and/or fold to allow the tether ring 110 to approach the proximal end 216 of the dispense cap 104. The material of the dispense cap 104 and/or dimensions of the arm 218 may affect the arm 218's capability of folding and/or bending. The tether ring 110 can attach to the tube body 106, thereby coupling the dispense cap 104 to the tube body 106 even when the dispense cap 104 is not attached to an open end 502 of the tube body 106 (sec FIG. 5A). In one example, the tether ring 110 is coupled to the tube body 106 by surrounding a perimeter of the tube body 106 between teeth 508 (described below with reference to FIGS. 5A-5C).

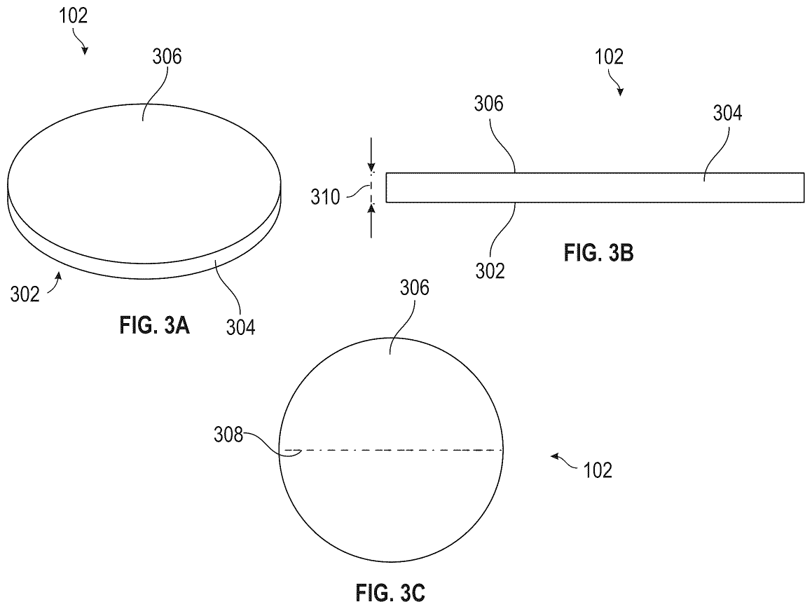

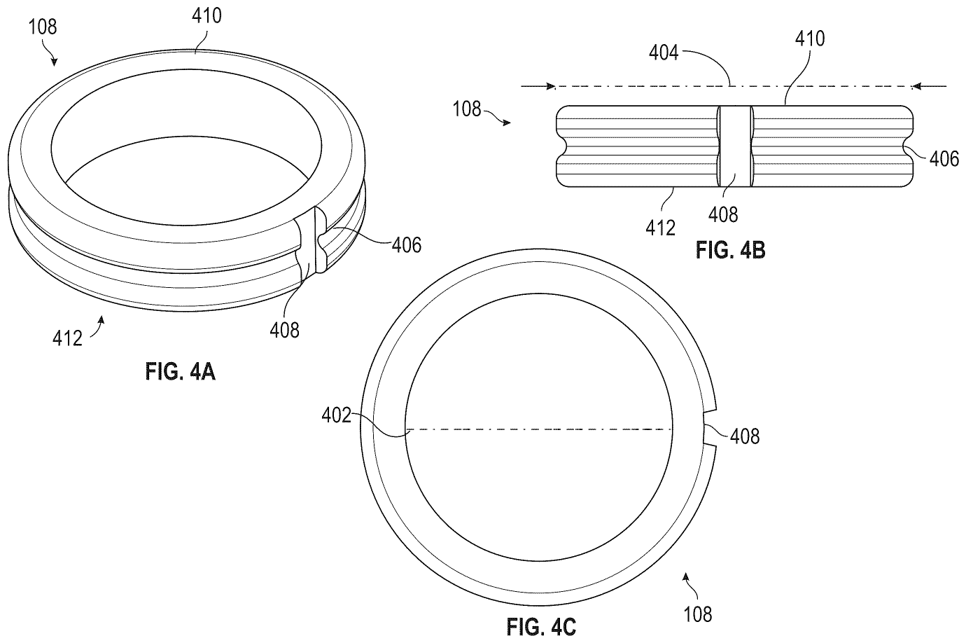

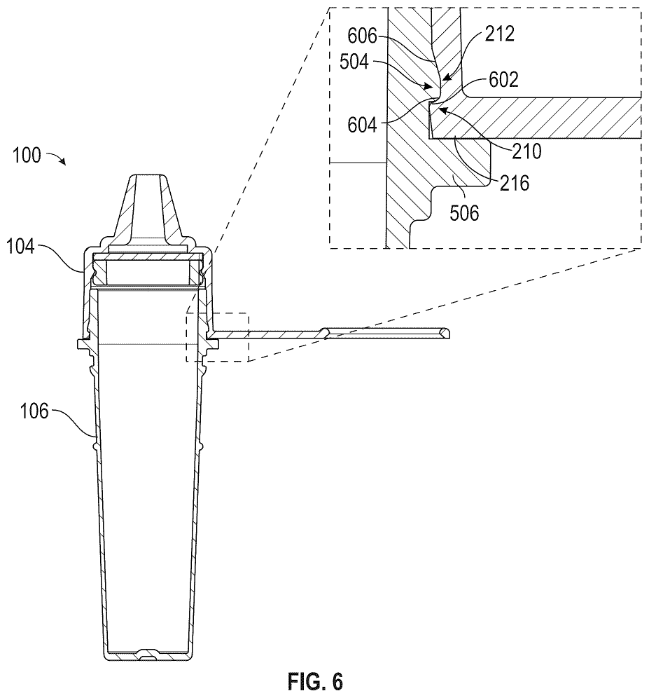

The cap body 224 may include the interior space 214. The cap body 224 may be sized and shaped to engage the open end 502 of the tube body 106. The interior surface of the cap body 224 may include a ridge 210 and an indentation 212 which can engage features of the tube body 106, as discussed with reference to FIG. 6 below. The ridge 210 and indentation 212 may run at least a portion of the circumference of the interior surface of the cap body 224. The cap body 224 may also include a second ridge 210 positioned proximate the filter cavity 208. The filter cavity 208 may be positioned between the second ridge 210 and the flow path 204. The filter cavity 208 may be sized such that the interior surface of the cap body 224 contacts a side surface 304 of the filter 102 (see FIGS. 3A-3C). The second ridge 210 can engage a groove 406 (see FIG. 4A-4B) of the retaining ring 108, thereby keeping the retaining ring 108 in position relative to the dispense cap 104. The second ridge 210 may run at least a portion of the circumference of the interior surface of the cap body 224.

In some embodiments, the dispense cap 104 may include a plastic. In some embodiments, the plastic may be a polypropylene or a high-density polyethylene. It may be desirable that the dimensions of the dispense cap 104 and the material of the dispense cap 104 are chosen such that the dispense cap 104 does not substantially deform, or minimally deforms, when the tube body 106 of the filter tube assembly 100 is compressed. In some examples, the Young's modulus of a material of the dispense cap 104 may be about 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 3000, 3010, 3020, 3030, 3040, 3050, 3060, 3070, 3080, 3090, or 4000 MPa, or any value or range within or bounded by any of these ranges or values, although values outside these values or ranges can be used in some cases. Additionally or alternatively, in some examples the Young's modulus of a material of the dispense cap 104 may be between about 1600 and 2000 MPa. Additionally or alternatively, in some examples the Young's modulus of a material of the dispense cap 104 may be between about 1700 and 1800 MPa. Additionally or alternatively, in some examples the Young's modulus of a material of the dispense cap 104 may be between about 1200 and 1500 MPa. Additionally or alternatively, in some examples the Young's modulus of a material of the dispense cap 104 may be between about 1300 and 1400 MPa. Additionally or alternatively, in some examples the Young's modulus of a material of the dispense cap 104 may be between about 900 and 1100 MPa. In some embodiments, the hardness of a material of the dispense cap 104 may be greater than the hardness of a material of the tube body 106 (for example, the Young's modulus of the material of the dispense cap 104 may be greater than that of the material of the tube body 106). In some examples, Young's modulus of a material of the dispense cap 104 may be greater than the Young's modulus of a material of the tube body 106 by at least about a factor of 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0, or any value or range within or bounded by any of these ranges or values, although values outside these values or ranges can be used in some cases. Additionally or alternatively, in some examples the Young's modulus of a material of the dispense cap 104 may be greater than the Young's modulus of a material of the tube body 106 by at least a factor of 2 (that is, at least twice as large).

In an alternative embodiment to those discussed with reference to FIGS. 2A-2F, the dispense cap 104 may include threading that can interact with a threaded portion of the tube body 106. In such embodiments, the user may secure the dispense cap 104 to the tube body 106 by rotating the dispense cap 104 relative to the tube body 106 to engage the threading. In such embodiments, the dispense cap 104 may not snap fit to the tube body 106.

### 2. Disc Filter

FIGS. 3A-3C illustrate views of the filter 102 included in a filter tube assembly 100 according to an embodiment of the present disclosure. FIG. 3A illustrates a perspective view of the filter 102. FIG. 3B illustrates a side view of the filter 102. FIG. 3C illustrates a top view of the filter 102. The filter 102 may be a disc filter, having a cylindrical shape. The disc filter can take the shape of a cylinder having a height less than its diameter. The filter 102 may include a first surface 302, a side surface 304, and a second surface 306. The first surface 302 and/or the second surface 306 may be circular in shape. The first surface 302 and the second surface 306 may have a diameter 308. When positioned within the dispense cap 104, the first surface 302 may be oriented toward the interior space 214 and the second surface 306 may be oriented toward the flow path 204 and/or the aperture 202.

In certain examples, the diameter 308 of the filter 102 may be approximately 8, 9, 10, 11, 12, 13, 14, 15, 16, 17, 18, 19, 20, 21, 22, 23, 24, 25, 26, 27, 28, 29, or 30 mm, or any value or range within or bounded by any of these ranges or values, although values outside these values or ranges can be used in some cases. Additionally or alternatively, in some examples the diameter 308 may be about 12 to 16 mm. Additionally or alternatively, in some examples the diameter 308 may be about 14 mm.

In certain examples, the surface area of the first surface 302 or second surface 306 may be approximately 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mm² or any value or range within or bounded by any of these ranges or values, although values outside these values or ranges can be used in some cases. Additionally or alternatively, in some examples the area of the first surface 302 or the second surface 306 may be about 100 to 200 mm². Additionally or alternatively, in some examples the area of the first surface 302 or the second surface 306 may be about 140 to 160 mm².

In certain examples, the thickness 310 of the filter may be approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, or any value or range within or bounded by any of these ranges or values, although values outside these values or ranges can be used in some cases. Additionally or alternatively, in some examples the thickness 310 may be about 0.8 to 1.2 mm. Additionally or alternatively, in some examples the thickness 310 may be about 1 mm. Additionally or alternatively, in some examples the thickness 310 may be about 1.3 to 1.8 mm. Additionally or alternatively, in some examples the thickness 310 may be about 1.6 mm.

In some examples, the filter may include a porous material. The size of the pores can affect which particles are filtered as the sample-containing fluid is propelled through the filter 102. It may be desirable that the size of the pores is small enough to filter particles that could interfere with a molecular assay. It may be desirable that the size of the pores is large enough to allow adequate volume and rate of fluid flow through the filter 102 and/or lower a risk that the filter 102 will become clogged with particles. The pore size (for example, average pore diameter) of the porous material may be 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μm or any value or range within or bounded by any of these ranges or values, although values outside these values or ranges can be used in some cases. Additionally or alternatively, in some examples the pore size may be about 5 to 25 μm. Additionally or alternatively, in some examples the pore size may be about 10 to 20 μm. Additionally or alternatively, in some examples the pore size may be about 50 to 120 μm. Additionally or alternatively, in some examples the pore size may be about 20 to 25 μm. Additionally or alternatively, in some examples the pore size may be about 5 to 15 μm. The porous material may include a plurality of tortuous paths. In some examples, the porous material may be hydrophobic. In some examples, the porous material may include a polyethylene (PE), a polypropylene (PP), or a polytetrafluoroethylene (PTFE). In some examples, the porous material may be hydrophilic. In some examples, the porous material may include a glass fiber or a melt blown polypropylene (PP). In some examples, the porous material may be a sintered porous material. In some examples, the porous material may be a sintered PE.

In one non-limiting embodiment, a filter includes a plurality of membranes, at least one membrane of the plurality of membranes having different properties (for example, different pore sizes and/or different hydrophobic/hydrophilic properties). The plurality of membranes can be arranged in a stack. One membrane can be arranged closer to the retaining ring (if one is provided) and serve as a prefilter. The membrane serving as a prefilter can include pores of a size and/or shape to retain large particles, while one or more downstream membranes can include pores of a size and/or shape to retain smaller particles. In one non-limiting example, a first tube assembly includes a filter including a single membrane, configured to dispense a first sample-containing fluid to be tested for a first analyte of interest. A second tube assembly includes a filter including two membranes, configured to dispense a second sample-containing fluid to be tested for a second analyte of interest different than the first analyte of interest. The second sample-containing fluid can include a crude matrix for which a two-level filtering format is optimal, relative to the first sample-containing fluid.

### 3. Retaining Ring

FIGS. 4A-4C illustrate views of the retaining ring 108 included in the filter tube assembly 100 according to an embodiment of the present disclosure. FIG. 4A illustrates a perspective view of the retaining ring 108. FIG. 4B illustrates a side view of the retaining ring 108. FIG. 4C illustrates a top view of the retaining ring 108.

The retaining ring 108 may include a groove 406, a distal surface 410, and a proximal surface 412. The retaining ring 108 may include a notch 408 in some embodiments. The retaining ring 108 may have an inner diameter 402 and an outer diameter 404. In some embodiments, the retaining ring 108 may include a plastic. In some embodiments, the retaining ring 108 may include a polypropylene.

The outer diameter 404 may be chosen such that the outer surfaces of the retaining ring 108 contact the surfaces of the interior space 214 of the dispense cap 104 when the retaining ring 108 is positioned within the dispense cap 104. The retaining ring 108 can be positioned within the dispense cap 104 to secure the filter 102 within the filter cavity 208 of the dispense cap 104. When the filter 102 and retaining ring 108 are positioned within the dispense cap 104, the distal surface 410 of the retaining ring 108 can contact the first surface 302 of the filter 102. The distal surface 410 of the retaining ring 108 can press the filter 102 towards and/or against the distal surface 226 of the filter cavity 208. In some examples, the outer diameter 404 may be approximately 8, 9, 10, 11, 12, 13, 14, 15, 16, 17, 18, 19, 20, 21, 22, 23, 24, 25, 26, 27, 28, 29, or 30 mm, or any value or range within or bounded by any of these ranges or values, although values outside these values or ranges can be used in some cases. Additionally or alternatively, in some examples the outer diameter 404 may be about 12 to 16 mm. Additionally or alternatively, in some examples the inner diameter 402 may be about 13 to 15 mm. Additionally or alternatively, in some examples the inner diameter 402 may be about 14 mm.

The groove 406 may run along at least a portion of the outer circumference of the retaining ring 108. The groove may be an indentation, depression, or the like in the outer circumference of the retaining ring 108. The groove 406 can engage the second ridge 210 of the dispense cap 104. The groove 406 can be shaped and/or dimensioned such that, when the retaining ring 108 is positioned within the dispense cap 104, the groove 406 firmly engages the second ridge 210. The groove 406 may be shaped and/or dimensioned such that, when engaged with the second ridge 210, the groove 406 and the second ridge 210 create a fluid-tight barrier.

In some embodiments, the notch 408 may run along at least a portion of the height of the outer surface of the retaining ring 108. The notch 408 may increase the flexibility of the retaining ring 108. For example, the increased flexibility due to inclusion of the notch 408 in the retaining ring 108 may allow the retaining ring 108 to be more easily positioned within the dispense cap 104, for example in a position where the second ridge 210 is engaged by the groove 406.

The inner diameter 402 may be chosen such that the retaining ring 108 has appropriate strength and/or flexibility. The inner diameter 402 may be chosen such that a sufficient area of the first surface 302 of the filter 102 is exposed to the sample-containing fluid when the tube body 106 is compressed, thus allowing for one or more streams of filtered sample-containing fluid to be propelled into the flow path 204. The one or more streams can be continuous streams of fluid propelled into the flow path 204. In some examples, the inner diameter 402 may be approximately 6, 7, 8, 9, 10, 11, 12, 13, 14, 15, 16, 17, 18, 19, or 20 mm, or any value or range within or bounded by any of these ranges or values, although values outside these values or ranges can be used in some cases. Additionally or alternatively, in some examples the inner diameter 402 may be about 8 to 12 mm. Additionally or alternatively, in some examples the inner diameter 402 may be about 9 to 11 mm. Additionally or alternatively, in some examples the inner diameter 402 may be about 10 to 11 mm. Additionally or alternatively, in some examples the inner diameter 402 may be about 10.5 mm.

### 4. Tube Body

FIGS. 5A-5C illustrate views of the tube body 106 included in the filter tube assembly 100 according to an embodiment of the present disclosure. FIG. 5A illustrates a perspective view of the tube body 106. FIG. 5B illustrates a side view of the tube body 106. FIG. 5C illustrates a cross-sectional side view of the tube body 106. FIG. 5D illustrates an angled top-down view of the tube body 106. The tube body 106 can include an open end 502, a snap fit ridge 504, a flange 506, teeth 508, a fill line 510, walls 512, a closed end 514, and an interior volume 516.

The interior volume 516 can hold a fluid, such as a sample-containing fluid. In some examples, the fluid is a buffer to which a sample is added, forming a sample-containing fluid. The dispense cap 104 can be positioned over the open end 502 of the tube body 106, such that the interior space 214 of the dispense cap 104 is fluidically connected to the interior volume 516, thereby allowing the filter 102 to be contacted by the sample-containing fluid if the tube body 106 is compressed and/or the filter tube assembly 100 is inverted. In some examples, the interior volume 516, in an uncompressed state, is approximately 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11, 11.5, 12, 12.5, 13, 13.5, 14, 14.5, or 15 mL or any value or range within or bounded by any of these ranges or values, although values outside these values or ranges can be used in some cases. Additionally or alternatively, in some examples the interior volume 516 is about 4 to 6 mL in an uncompressed state. Additionally or alternatively, in some examples the interior volume 516 is about 5 mL. In some examples, the tube body 106 can hold approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11, 11.5, 12, 12.5, 13, 13.5, 14, 14.5, or 15 mL of sample-containing fluid or any value or range within or bounded by any of these ranges or values, although values outside these values or ranges can be used in some cases. Additionally or alternatively, in some examples the tube body 106 can hold about 2 to 4 mL of sample-containing fluid. Additionally or alternatively, in some examples the tube body 106 can hold about 3 mL of sample-containing fluid. In some examples, the tube body 106 can include approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mL of headspace volume (for example, a volume within the tube body 106 that is not occupied by fluid when the buffer or sample-containing fluid is present within the tube body 106) or any value or range within or bounded by any of these ranges or values, although values outside these values or ranges can be used in some cases. Additionally or alternatively, in some examples the tube body 106 can include about 1 to 3 mL of headspace volume. Additionally or alternatively, in some examples the tube body 106 can include about 2 mL of headspace volume.

In some embodiments, the interior volume 516 of the tube body 106 can be reduced in order to propel the sample-containing fluid from the filter tube assembly 100 to a test device, for example for performing a molecular assay, such as but not limited to nucleic acid amplification. Reduction of the interior volume 516 can be accomplished in several ways. In a first embodiment, illustrated in FIGS. 5A-5C, the material of the tube body 106 is flexible enough to allow the user to compress the walls 512 of the tube body 106 to propel one or more streams of filtered sample-containing fluid to the test device through the aperture 202 of the dispense cap 104. The one or more streams can be continuous streams of fluid propelled through the aperture 202. The flexibility can arise from a combination of thickness of the walls 512 and modulus (for example, a Young's modulus) of the material included in the tube body 106. For example, the thickness of material near the open end 502 and at the closed end 514 may be equal to or larger than the thickness of the walls 512. This combination of thickness of walls 512 and material of the tube body 106 can be chosen so that the tube body 106 is compressible by a user. In a second embodiment, the tube body 106 can have thin sections in the walls 512, running axially and/or radially, that give the tube body 106 hinge points where the walls 512 can flex while other portions of the walls 512 are thicker and/or stiffer. The user can then compress the tube body 106, which flexes at the thin hinge points thus reducing the interior volume 516 and propelling the sample-containing fluid out through the filter 102 and flow path 204 and out the aperture 202 of the dispense cap 104, without the entire wall 512 being thin enough to flex. Other approaches to propel a stream or other volume of sample-containing fluid from the tube body 106 through the dispense cap 104 are possible. In other embodiments, the tube body 106 may not require compression or squeezing to dispense fluid from the filter tube assembly 100 and may dispense drops of fluid upon inversion of the filter tube assembly 100.

In some embodiments, the thickness 518 of the material of the tube body 106 near the open end 502 may be equal to or larger than that of the thickness 520 of the walls 512. In some embodiments, the thickness 522 of the material of the closed end 514 may be equal to or larger than the thickness 520 of the walls 512. In some examples, the thickness 520 of the walls 512 may be about 0.20, 0.25, 0.30, 0.35, 0.40, 0.45, 0.50, 0.55, 0.60, 0.65, 0.70, 0.75, 0.80, 0.85, 0.90, 0.95, or 1.0, mm or any value or range within or bounded by any of these ranges or values, although values outside these values or ranges can be used in some cases. Additionally or alternatively, in some examples the thickness 520 is about 0.40 to 0.60 mm. Additionally or alternatively, in some examples the thickness 520 is about 0.50 to 0.60 mm. In some examples, the thickness 518 near the open end 502 may be about 0.60, 0.65, 0.70, 0.75, 0.80, 0.85, 0.90, 0.95, 1.0, 1.1, 1.2, 1.3, 1.4, or 1.5 mm or any value or range within or bounded by any of these ranges or values, although values outside these values or ranges can be used in some cases. Additionally or alternatively, in some examples the thickness 518 is about 0.90 to 1.1 mm. Additionally or alternatively, in some examples the thickness 518 is about 1.0 to 1.1 mm. In some examples, the thickness 522 of the closed end 514 may be about 0.60, 0.65, 0.70, 0.75, 0.80, 0.85, 0.90, 0.95, 1.0, 1.1, 1.2, 1.3, 1.4, or 1.5 mm or any value or range within or bounded by any of these ranges or values, although values outside these values or ranges can be used in some cases. Additionally or alternatively, in some examples the thickness 522 is about 0.70 to 1.0 mm. Additionally or alternatively, in some examples the thickness 522 is about 0.80 to 0.90 mm.

In some embodiments, the tube body 106 may include a plastic. In some embodiments, the plastic may be a polyethylene. In some embodiments, the plastic may be a low-density polyethylene (LDPE). In some examples, the Young's modulus of a material of the tube body 106 may be about 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 MPa, or any value or range within or bounded by any of these ranges or values, although values outside these values or ranges can be used in some cases. Additionally or alternatively, in some examples the Young's modulus of a material of the tube body 106 may be between about 200 and 700 MPa Additionally or alternatively, in some examples the Young's modulus of a material of the tube body 106 may be between about 200 and 300 MPa. Additionally or alternatively, in some examples the Young's modulus of a material of the tube body 106 may be between about 90 and 200 MPa. Additionally or alternatively, in some examples the Young's modulus of a material of the tube body 106 may be between about 800 and 950 MPa. In some embodiments, the hardness of a material of the tube body 106 may be lower than a hardness of a material of the dispense cap 104 (for example, the Young's modulus of the material of the tube body 106 may be lower than that of the material of the dispense cap 104).

The material included in the tube body 106 may be optically transparent, for example transparent to visible light. In some examples, the transparency of the material of the tube body 106 may allow a user to view the volume of sample-containing fluid and/or buffer held within the interior volume 516. The fill line 510 can indicate to a user whether there is an adequate amount of sample-containing fluid and/or buffer within the interior volume 516 of the tube body 106. In some embodiments, as illustrated in FIGS. 5A-5C, the fill line 510 can be a raised feature molded into the walls 512. In some embodiments, the fill line 510 can be a depression molded into the walls 512. In some embodiments, the fill line 510 can be printed on the walls 512, for example with an ink or other marking material. In some embodiments, the tube body 106 can include more than one fill line 510. In such embodiments, each fill line 510 may indicate a different volume. When the filter tube assembly 100 is oriented such that the dispense cap 104 is pointed upward and the closed end 514 of the tube body 106 is pointed downward, the user may be able to compare the vertical position of the upper surface of the buffer and/or sample-containing fluid within the interior volume 516 relative to the position of the fill line 510. In some examples, the fill line 510 can indicate whether there is a minimum required volume of sample-containing fluid present to perform a molecular assay. In some examples where a buffer fluid is stored within the interior volume 516 over a period of days, weeks, months, and/or years, the fill line 510 can indicate whether buffer volume has been lost beyond a threshold indicated by the fill line 510, for example due to evaporation, leakage, and/or escape from the tube body 106.

The dispense cap 104 may be positioned on and/or over the open end 502. The snap fit ridge 504 of the tube body 106 may engage the indentation 212 of the dispense cap 104. In some examples, the indentation 212 and the snap fit ridge 504 may matingly engage to create a snap-fit connection (described below with reference to FIG. 6). When positioned on and/or over the open end 502, the proximal end 216 of the dispense cap 104 may abut the flange 506. In some embodiments, the proximal end 216 need not abut the flange 506 to confirm an effective seal is formed between the dispense cap 104 and the tube body 106. The flange 506 may thereby prevent further translation of the dispense cap 104 in the direction of the closed end 514 of the tube body 106.

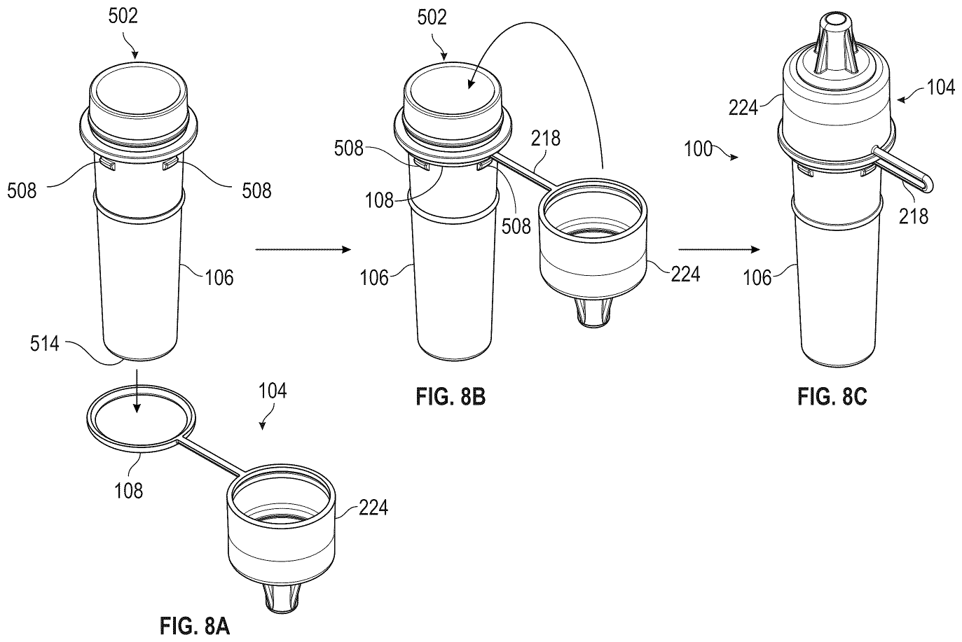

The teeth 508 may be positioned on an outer surface of the tube body 106. The teeth 508 may be positioned closer to the open end 502 than the closed end 514. In some examples, the teeth 508 are positioned proximate the flange 506. There may be two, three, four, or more teeth positioned along a circumference of the outer surface of the walls 512. FIGS. 5A-5C illustrate an embodiment including four teeth 508 positioned equidistant about a circumference of the outer surface of the walls 512. The teeth 508 can engage the tether ring 110 of the dispense cap 104 (see FIG. 8A-8B, which illustrate positioning of the retaining ring 108 of the dispense cap to the teeth 508).

### 5. Assembly Including Dispense Cap, Disc Filter, and Tube Body

FIG. 6 illustrates a cross-sectional view of the filter tube assembly 100 according to an embodiment of the present disclosure. When the dispense cap 104 is attached to the open end 502 of the tube body 106, the indentation 212 and the ridge 210 of the dispense cap 104 can engage the snap fit ridge 504 of the tube body 106. When the dispense cap 104 is attached to the tube body 106, for example by snap-fit connection, the proximal end 216 of the dispense cap 104 can abut the flange 506 of the tube body 106. In some embodiments, the proximal end 216 need not abut the flange 506 to confirm an effective seal is formed between the dispense cap 104 and the tube body 106.

The shape of the indentation 212, the ridge 210, and snap fit ridge 504 can each affect the amount of force to attach the dispense cap 104 to the tube body 106 and/or remove the dispense cap 104 from the tube body 106. For instance, the shape and/or angle of indentation curvature 602, ridge curvature 604, and slope 606 can each be altered to increase and/or decrease the force to snap the dispense cap 104 onto the tube body 106 and/or remove the dispense cap 104 from the tube body 106. For example, as illustrated in FIG. 6, the slope 606 can be a relatively shallow slope. When the slope 606 is relatively shallow, the force needed to attach (in this non-limiting example, snap) the dispense cap 104 onto the tube body 106 may be relatively low. Increasing the steepness of the slope 606 may increase the force to attach the dispense cap 104 to the tube body 106. In some examples, the force exerted to attach the dispense cap 104 to the tube body 106 may be less than the force exerted to remove the dispense cap 104 from the tube body 106. In some examples, the force exerted to attach the dispense cap 104 to the tube body 106 can be about 15, 20, 25, 30, 35, 40, 45, 50, 55, 60, 65, 70, 75, 80, 85, 90, 95, 100, 105, 110, 115, or 120 N, or any value or range within or bounded by any of these ranges or values, although values outside these values or ranges can be used in some cases. Additionally or alternatively, in some examples the exerted to attach the dispense cap 104 to the tube body 106 can be about 40 to 80 N. Additionally or alternatively, in some examples the force exerted to attach the dispense cap 104 to the tube body 106 can be about 50 to 70 N. Additionally or alternatively, in some examples the force exerted to attach the dispense cap 104 to the tube body 106 can be about 55 to 65 N. Additionally or alternatively, in some examples the force exerted to attach the dispense cap 104 to the tube body 106 can be about 55 to 60 N.

The shape, slope, and/or curvature of indentation curvature 602 and ridge curvature 604 can affect the force to remove the dispense cap 104 from the tube body 106. The indentation curvature 602 and ridge curvature 604 may be shaped to resist detachment of the dispense cap 104 from the tube body 106. For example, as illustrated in FIG. 6, the indentation curvature 602 and ridge curvature 604 can both be relatively steep, resulting in the need to apply a relatively high force to remove the dispense cap 104 from the tube body 106. The indentation curvature 602 and/or ridge curvature 604 can be formed such that the dispense cap 104 and tube body 106 remain attached even when pressure within the interior volume 516 of the tube body 106 is well above atmospheric pressure due to, for example, a compression of the tube body 106 by a user. In some examples, the force exerted to remove the dispense cap 104 from the tube body 106 can be at least about 80, 85, 90, 95, 100, 105, 110, 115, 120, 125, 130, 135, 140, 145, 150, 155, 160, 165, 170, 175, 180, 185, 190, 195, or 200 N, or any value or range within or bounded by any of these ranges or values, although values outside these values or ranges can be used in some cases. Additionally or alternatively, in some examples the force exerted to remove the dispense cap 104 from the tube body 106 can be at least about 125 N. Additionally or alternatively, in some examples the force exerted to remove the dispense cap 104 from the tube body 106 can be at least about 150 N. It may be desirable that the force to attach the dispense cap 104 to the tube body 106 be less than the force to remove the dispense cap 104 from the tube body 106.

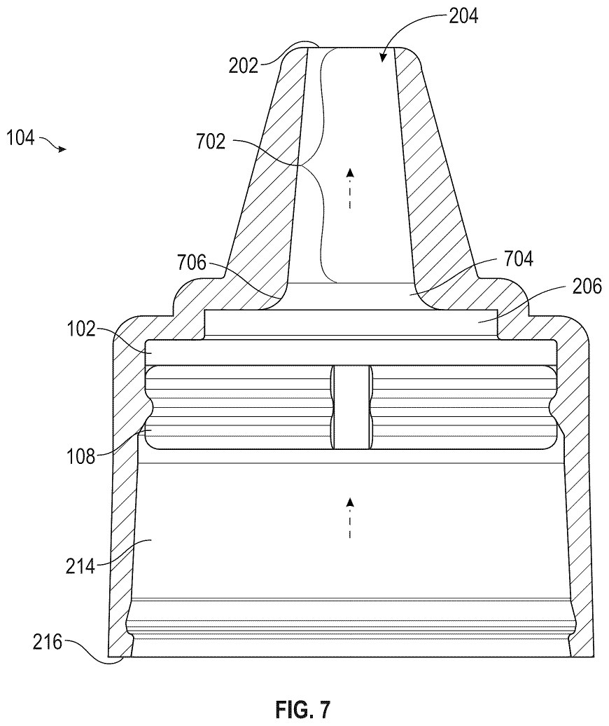

FIG. 7 illustrates a cross-sectional view of the filter 102 and retaining ring 108 positioned within the dispense cap 104. Dashed arrows indicate the direction (for example, in the distal direction, away from the proximal end 216 of the dispense cap 104) of fluid flow through the dispense cap 104 when the tube body 106 of the filter tube assembly 100 is compressed. When the tube body 106 of the filter tube assembly 100 is compressed, the sample-containing fluid can flow from the interior space 214 and/or interior volume 516 (which are fluidically connected) through the hollow space defined by the inner diameter 402 of the retaining ring 108, through the filter 102, through the flow path 204 (which includes the transition zone 206, the intermediate portion 704, and the distal portion 702), and out the aperture 202.

As previously discussed, the transition zone 206 can reduce back pressure (that is, high pressure in the interior space 214 relative to that of the flow path 204) acting on the filter 102. The transition zone 206 provides a space with a diameter not much smaller than that of the filter 102 (for example, the diameter of the first surface 302 or second surface 306). In some examples, the diameter of the filter 102 is about 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or 2.00 times larger than the diameter of the transition zone 206, or any value or range within or bounded by any of these ranges or values, although values outside these values or ranges can be used in some cases. Additionally or alternatively, in some examples the diameter of the filter 102 is about 1.2-1.6 times larger than the diameter of the transition zone 206. Additionally or alternatively, in some examples the diameter of the filter 102 is about 1.3-1.5 times larger than the diameter of the transition zone 206. Additionally or alternatively, in some examples the diameter of the filter 102 is about 1.35-1.45 times larger than the diameter of the transition zone 206. Additionally or alternatively, in some examples the diameter of the filter 102 is about 1.4 times larger than the diameter of the transition zone 206.

The shape and/or dimensions of the distal portion 702 and intermediate portion 704 of the flow path 204 may also affect pressure on the filter 102. It may be desirable that the interior surface of the dispense cap 104 defining the intermediate portion 704, referred to as curved portion 706, include a gradual curve such that the diameter of the flow path 204 does not suddenly decrease from the transition zone 206 to the distal portion 702. A gradual change in diameter of the flow path 204 may ensure that there are no sharp increases or decreases in pressure of the sample-containing fluid. The interior walls of the dispense cap 104 defining the distal portion 702 may be shallowly sloped, such that a diameter at a proximal portion of the distal portion 702 (for example, the portion proximate to the intermediate portion 704) is larger than a diameter of the aperture 202.

The ratio of the surface area of the filter 102 to the size of the interior volume 516 may affect the maximum flow rate of the sample-containing fluid out of the filter tube assembly 100 upon compression of the tube body 106. It may be desirable that the maximum flow rate is high enough such that the filter tube assembly 100 can dispense the total volume of filtered sample-containing fluid for the molecular assay within no more than one, two, three, or four compressions of the tube body 106 as previously discussed. In some examples, the ratio a:v of the surface area of the first surface 302 of the filter 102 to the interior volume 516 of the tube body 106 may be about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 m⁻¹, or any value or range within or bounded by any of these ranges or values, although values outside these values or ranges can be used in some cases. Additionally or alternatively, in some examples the ratio a:v of the surface area of the first surface 302 of the filter 102 to the interior volume 516 of the tube body 106 is about 20-40 m⁻¹. Additionally or alternatively, in some examples the ratio a:v of the surface area of the first surface 302 of the filter 102 to the interior volume 516 of the tube body 106 is about 25-35 m⁻¹. Additionally or alternatively, in some examples the ratio a:v of the surface area of the first surface 302 of the filter 102 to the interior volume 516 of the tube body 106 is about 30 m⁻¹. The maximum flow rate may be affected by the inner diameter 402 of the retaining ring 108, as the surface area of the first surface 302 of the filter 102 that contacts the distal surface 410 of the retaining ring 108 may be inaccessible to sample-containing fluid propelled by a compression of the tube body 106. That is to say, the effective surface area of the first surface 302 of the filter 102 may be approximately equal to the surface area of the first surface 302 of the filter 102 exposed within the opening of the retaining ring 108 defined by the inner diameter 402. In some examples, the ratio a:v of the exposed surface area of the first surface 302 of the filter 102 to the interior volume 516 of the tube body 106 may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 m⁻¹, or any value or range within or bounded by any of these ranges or values, although values outside these values or ranges can be used in some cases. Additionally or alternatively, in some examples the ratio a:v of the exposed surface area of the first surface 302 of the filter 102 to the interior volume 516 of the tube body 106 is about 10-25 m⁻¹. Additionally or alternatively, in some examples the ratio a:v of the exposed surface area of the first surface 302 of the filter 102 to the interior volume 516 of the tube body 106 is about 15-20 m⁻¹. Additionally or alternatively, in some examples the ratio a:v of the exposed surface area of the first surface 302 of the filter 102 to the interior volume 516 of the tube body 106 is about 17 m⁻¹.

FIG. 8A-8C illustrates example steps of attaching the retaining ring 108 to the tube body 106 and closing the open end 502 using the dispense cap 104 according to the present disclosure. FIG. 8A illustrates the closed end 514 of the tube body 106 inserting into the opening of the retaining ring 108. The retaining ring 108 may be engaged and/or secured by the teeth 508, as illustrated in FIG. 8B. In this non-limiting example, the retaining ring 108 is held in position relative to the tube body 106 by the teeth 508. The teeth 508 can impede upward or downward movement of the retaining ring 108 relative to the tube body 106, while still allowing the retaining ring 108 to rotate about a longitudinal axis of the tube body 106. The cap body 224 may be moved, for example by bending and/or folding the arm 218, to attach to and/or close the open end 502 of the tube body 106, as indicated by the directional arrow in FIG. 8B. FIG. 8C illustrates the filter tube assembly 100 when the retaining ring 108 and cap body 224 are attached to the tube body 106.

### 6. Travel Cap and Other Components for Long-Term Fluid Storage

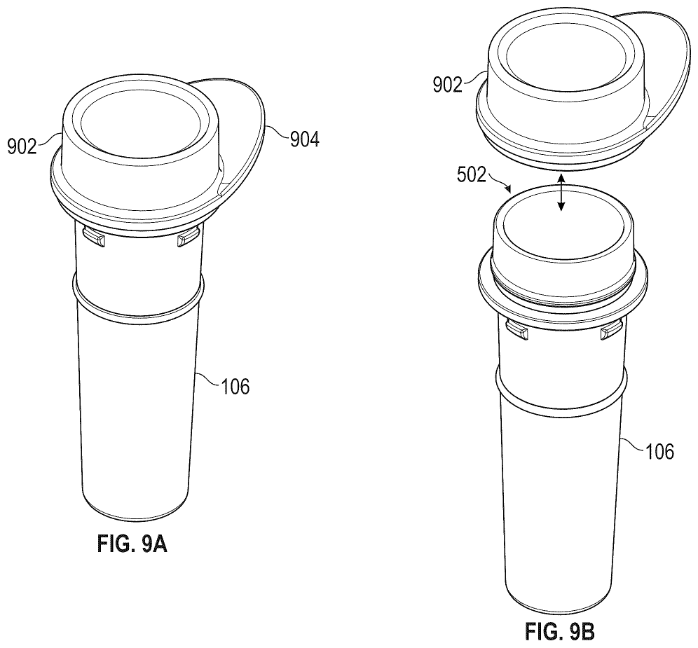

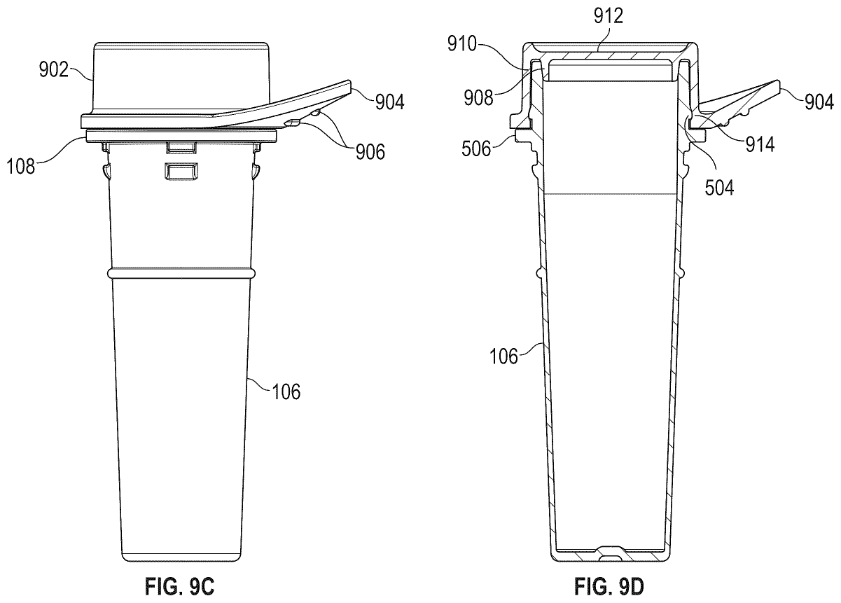

FIGS. 9A-9B illustrate an example travel cap 902 attached to the tube

body 106 according to the present disclosure. FIG. 9A illustrates a perspective view of the travel cap 902 attached to the tube body 106. FIG. 9B shows an exploded view of the travel cap 902 and tube body 106. FIG. 9C illustrates a side view of the travel cap 902 attached to the tube body 106. FIG. 9D illustrates a cross-sectional side view of the travel cap 902 attached to the tube body 106. In some embodiments, a travel cap 902 may be used to cover the open end 502. The travel cap 902 may be used, for example, if the tube body 106 is used for storing buffer prior to filtering a sample using the filter tube assembly 100. The travel cap 902 can prevent and/or inhibit fluid contained within the tube body 106 from leaking, releasing, evaporating, or the like. By closing the open end 502 of the tube body 106 with the travel cap 902, loss of a fluid held within the tube body 106, for example a buffer, may be inhibited and/or prevented. The travel cap 902 includes a thumb tab 904. The thumb tab 904 can include furrows 906. The thumb tab 904 may provide leverage for removing the travel cap 902 from the tube body 106. The furrows 906 of the thumb tab 904 may provide additional grip, allowing for easier removal of the travel cap 902 from the tube body 106.

The travel cap 902 may include a collar 908 that can create a fluid-tight barrier (for example, a plug seal) against a portion of the open end 502 of the tube body 106. The collar 908 may extend from the cap top 912. The collar 908 may contact an interior circumference of the tube body 106 at the open end 502, thereby creating a fluid-tight seal. When the travel cap 902 is attached to the tube body 106, the upper portion of the tube body 106 (for example, the open end 502) may be wedged between the collar 908 and the travel cap wall 910. The travel cap 902 may include an indentation 914 running along at least a portion of the circumference of the interior surface of the travel cap 902 that can engage the snap fit ridge 504 of the tube body 106. When the travel cap 902 is attached to the tube body 106, the travel cap 902 may abut the flange 506 of the tube body 106. In some embodiments, the thickness of the cap top 912 may be substantially the same as at least a portion of the travel cap wall 910.

In alternative embodiment to those discussed with reference to FIGS. 9A and 9B, the travel cap 902 may include threading that can interact with a threaded portion of the tube body 106. In such embodiments, the user may secure the travel cap 902 to the tube body 106 by rotating the travel cap 902 relative to the tube body 106 to engage the threading. In such embodiments, the travel cap 902 may not snap fit to the tube body 106.

In some embodiments, particularly those where fluid, for example buffer fluid, is stored within the tube body 106 for an extended period (for example, for days, weeks, months, and/or years) before the filter tube assembly 100 is used to filter a sample-containing fluid, a seal may be affixed to the open end 502, thereby preventing evaporation, contamination, or the like of the buffer within the tube body 106. As an illustrative example, the seal may be a foil heat-pressed seal. Alternatively, induction sealing may be used to bond the seal to the open end 502 of the tube body 106.

B. Filter Tube Assembly with Disc Filter and Single-Component Tube Body and Dispense Cap

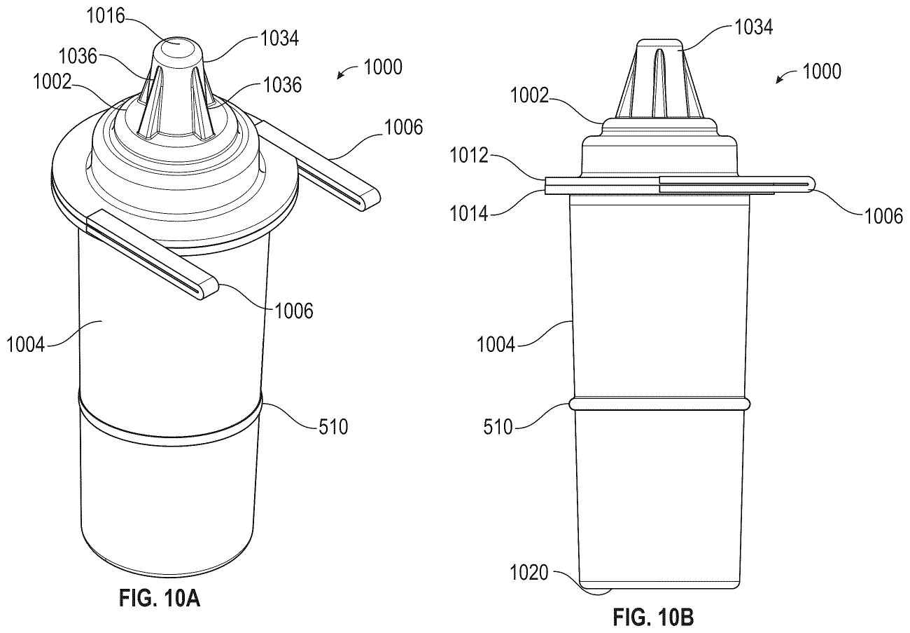

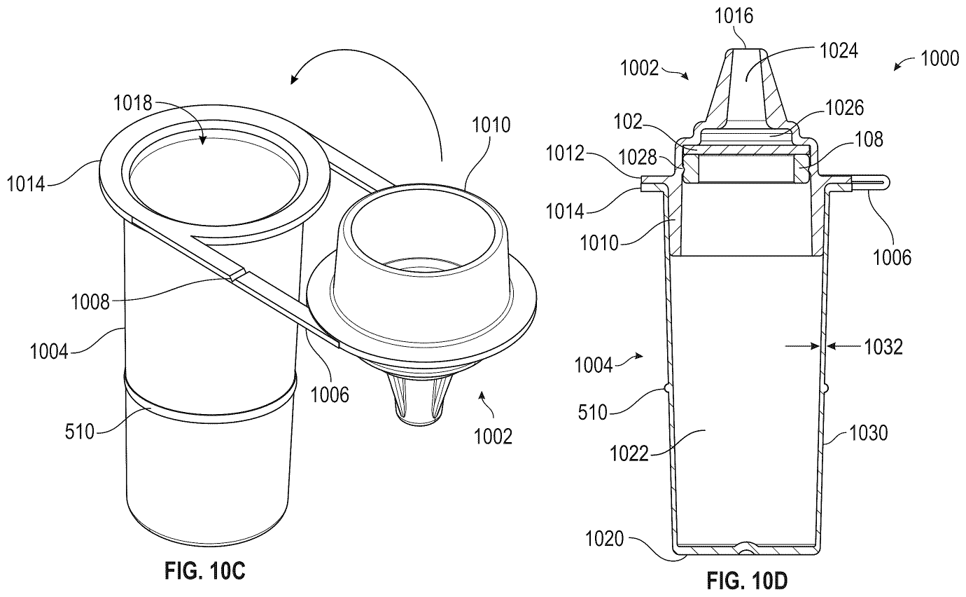

Embodiments of filter tube assemblies according to the present disclosure can include single-piece assemblies. For example, in some embodiments, a filter tube assembly may include a dispense cap and a tube body that are a single component, for example molded as a single piece of plastic. FIG. 10A-10D illustrate an example filter tube assembly 1000 that can be used for preparing a sample for a molecular assay according to an embodiment of the present disclosure, where the dispense cap 1002 and the tube body 1004 are a single component. FIG. 10A illustrates a perspective view of the filter tube assembly 1000. FIG. 10B illustrates a side view of the filter tube assembly 1000. FIG. 10C illustrates a perspective view of the filter tube assembly 1000 with a directional arrow showing motion of the dispense cap 1002 to close an open end 1018 of the tube body 1004. FIG. 10D illustrates a cross-sectional side view of the filter tube assembly 1000. The filter tube assembly 1000 can propel volumes of filtered sample-containing fluid in no more than one, two, three, or four compressions in accordance with the present disclosure (for example, with reference to filter tube assembly 100). It will be understood that filter tube assembly 1000 can include various features described herein in accordance with the present disclosure, including features described with reference to the filter tube assembly 100.

The filter tube assembly 1000 may include arms 1006 connecting the dispense cap 1002 and the tube body 1004. In some embodiments, the filter tube assembly 1000 includes two arms. In some embodiments, the filter tube assembly 1000 may only include one arm. The arms 1006 may include a notch 1008 that can allow the arms 1006 to bend and/or fold. The dispense cap 1002 may include a collar 1010, a cap flange 1012, an aperture 1016, a flow path 1024, a transition zone 1026, and a ridge 1028. The dispense cap 1002 can engage the retaining ring 108 and the filter 102 such that the filter 102 is positioned between an interior volume 1022 of the tube body 1004 and the flow path 1024. The ridge 1028 may run along at least a portion of the interior circumference of the dispense cap 1002. The ridge 1028 can engage the groove 406 of the retaining ring 108, thereby securing the retaining ring 108 and the filter 102 within the dispense cap 1002. The flow path 1024 and/or transition zone 1026 may be sized and shaped in accordance with the present disclosure (for example, with reference to flow path 204 and transition zone 206). In some embodiments, the dispense cap 1002 can include buttresses 1036 positioned about the nozzle 1034, which can support and/or stabilize the nozzle 1034. In some embodiments, the dispense cap 1002 may not include such buttresses.

The tube body 1004 may include a tube flange 1014, a fill line 510, an open end 1018, a closed end 1020, walls 1030, and an interior volume 1022. The tube body 1004 and/or interior volume 1022 may be sized and/or shaped in accordance with the present disclosure (for example, with reference to tube body 106 and/or interior volume 516).

The material included in the tube body 1004 may be at least partially optically transparent, for example at least partially transparent to visible light. In some examples, the transparency of the material of the tube body 1004 may allow a user to view the volume of sample-containing fluid and/or buffer held within the interior volume 1022. The fill line 510 can indicate to a user whether there is an adequate amount of sample-containing fluid and/or buffer within the interior volume 1022 of the tube body 1004. In some embodiments, as illustrated in FIGS. 10A-10D, the fill line 510 can be a raised feature molded into the walls 1030 of the tube body 1004. In some embodiments, the fill line 510 can be a depression molded into the walls 1030. In some embodiments, the fill line 510 can be printed on the walls 1030, for example with an ink or a marking material. In some embodiments, the tube body 1004 can include more than one fill line 510. In such embodiments, each fill line 510 may indicate a different volume. When the filter tube assembly 1000 is oriented such that the dispense cap 1002 is pointed upward and the closed end 1020 of the tube body 1004 is pointed downward, the user may be able to compare the vertical position of the upper surface of the buffer and/or sample-containing fluid within the interior volume 1022 relative to the position of the fill line 510. In some examples, the fill line 510 can indicate whether a minimum required volume of sample-containing fluid is present to perform a molecular assay. In some examples where a buffer fluid is stored within the interior volume 516 over a period of days, weeks, months, and/or years, the fill line 510 can indicate whether buffer volume has been lost beyond a threshold indicated by the fill line 510, for example due to evaporation or escape from the tube body 1004. In some embodiments where fluid, for example buffer fluid, is stored within the tube body 1004 for an extended period (for example, for days, weeks, months, and/or years) before the filter tube assembly 1000 is used to filter a sample-containing fluid, a seal may be affixed to the open end 1018 to create a fluid-tight barrier, thereby preventing evaporation, contamination, or the like of the buffer within the tube body 1004. As an illustrative example, the seal may be a foil heat-pressed seal or an induction seal. Alternatively, in some embodiments, the filter tube assembly 1000 may include a travel cap that can create a fluid-tight barrier with the open end 1018 of the tube body 1004.

In some embodiments, the interior volume 1022 of the tube body 1004 can be reduced in order to propel the sample-containing fluid from the filter tube assembly 1000 to a test device, for example for performing a molecular assay, such as but not limited to nucleic acid amplification. Reduction of the interior volume 1022 can be accomplished in several ways. In a first embodiment, illustrated in FIGS. 10A-10D, the material of the tube body 1004 is flexible enough to allow the user to compress the walls 1030 of the tube body 1004 to propel one or more streams of filtered sample-containing fluid to the test device through the aperture 1016 of the dispense cap 1002. The one or more streams can be continuous streams of fluid propelled through the aperture 1016. The flexibility can arise from a combination of thickness 1032 of the walls 1030 and modulus (for example, a Young's modulus) of the material included in the tube body 1004. For example, the thickness 1032 of the walls 1030 may be less than the thickness of portions of the dispense cap 1002. This combination of the thickness 1032 of walls 1030 and material of the tube body 1004 can be chosen so that the tube body 1004 can be compressed by a user. In a second embodiment, the tube body 1004 can have thin sections in the walls 1030, running axially and/or radially, that give the tube body 1004 hinge points where the walls 1030 can flex while other portions of the walls 1030 are thicker and/or stiffer. The user can then compress the tube body 1004, which flexes at the thin hinge points thus reducing the interior volume 1022 and propelling the sample-containing fluid out through the filter 102 and flow path 1024 and out the aperture 1016 of the dispense cap 1002 without the entire wall 1030 being thin enough to flex. Other configurations to propel a stream or other volume of sample-containing fluid from the tube body 1004 through the dispense cap 104 are possible. In other embodiments, the tube body 1004 may not require compression or squeezing to dispense fluid from the container and may dispense drops of fluid upon inversion of the filter tube assembly 1000.

In some embodiments, the thickness of the material of the closed end 1020 may be equal to or larger than the thickness 1032 of the walls 1030. In some examples, the thickness 1032 of the walls 1030 may be about 0.20, 0.25, 0.30, 0.35, 0.40, 0.45, 0.50, 0.55, 0.60, 0.65, 0.70, 0.75, 0.80, 0.85, 0.90, 0.95, or 1.0, mm or any value or range within or bounded by any of these ranges or values, although values outside these values or ranges can be used in some cases. Additionally or alternatively, in some examples the thickness 1032 is about 0.40 to 0.60 mm. Additionally or alternatively, in some examples the thickness 1032 is about 0.45 to 0.55 mm. Additionally or alternatively, in some examples the thickness 1032 is about 0.51 mm.

The dispense cap 1002 may include a collar 1010 that can create a fluid-tight barrier against a portion of the open end 1018 of the tube body 1004. The collar 1010 may contact an interior circumference of the open end 1018 of the tube body 1004, thereby creating a fluid-tight seal. When the dispense cap 1002 is attached to the open end 1018 of the tube body 1004, the collar 1010 may cause the walls of the tube body 1004 near the open end 1018 to flex in an outward direction. When the dispense cap 1002 is attached to the open end 1018 of the tube body 1004, the cap flange 1012 of the dispense cap 1002 may abut the tube flange 1014 of the tube body 1004. In some embodiments, the cap flange 1012 need not abut the tube flange 1014 to confirm an effective seal is formed between the dispense cap 1002 and the tube body 1004.

C. Filter Tube Assembly with Cup Filter and Single-Component Tube Body and Dispense Cap

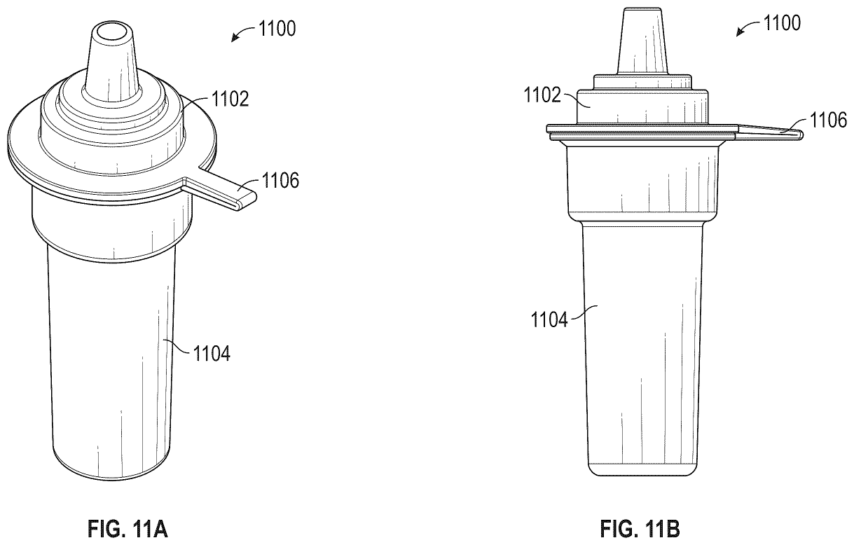

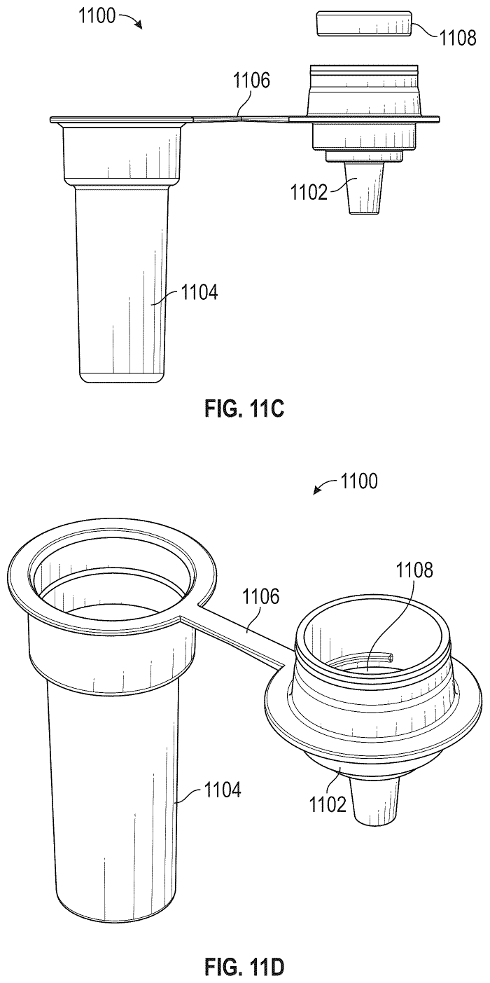

As discussed previously, embodiments of filter tube assemblies according to the present disclosure can include assemblies having a single component including a tube body and a dispense cap. For example, in some embodiments, a filter tube assembly may include a dispense cap and a tube body may be molded as a single piece of plastic. FIGS. 11A-11D illustrate an example filter tube assembly that can be used for preparing a sample for a molecular assay according to an embodiment of the present disclosure. FIG. 11A illustrates an example filter tube assembly 1100 in an angled view. FIG. 11B illustrates the example filter tube assembly 1100 in a side view. FIG. 11C illustrates the filter tube assembly 1100 in an exploded view side view. FIG. 11D illustrates the filter tube assembly 1100 in an angled view where the dispense cap 1102 is not secured to the tube body 1104.

The filter tube assembly 1100 can include a filter 1108 (for example, a cup filter), a dispense cap 1102, and a tube body 1104. In some embodiments, the filter tube assembly 1100 can include an arm 1106 that tethers the tube body 1104 to the dispense cap 1102. The filter tube assembly 1100 may be used to prepare a sample-containing fluid for use in a molecular assay. The tube body 1104 may be capable of holding a fluid, for example a buffer or a sample-containing fluid. When open, the tube body 1104 may be capable of receiving a sample, for example a swab on which a sample has been collected. When the dispense cap 1102 is attached to the tube body 1104 and the tube body 1104 is compressed, the filter tube assembly 1100 can propel the sample-containing fluid from an interior volume of the tube body 1104 through the filter 1108 and out from the dispense cap 1102.

In some examples, the filter tube assembly 1100 is capable of propelling the filtered sample-containing fluid out the dispense cap 1102 in one or more streams (for example, at a significantly higher dispense rate than a dropper). In some embodiments of the filter tube assembly according to the present disclosure, the dispense cap is configured to propel a continuous stream of filtered sample-containing fluid through the aperture during each compression of the tube body. Advantageously, embodiments of the filter tube assembly can thus deliver high volumes of filtered sample-containing fluid out of the aperture rapidly, precisely, and with minimal user interaction, thereby minimizing the risk that the filtered sample-containing fluid will become exposed to environmental or user-derived contaminants, degraded, or compromised before the filtered sample-containing fluid is delivered to a test device where a molecular assay is performed. In one non-limiting embodiment described below, a filter tube assembly according to the present disclosure is configured to propel about 2.5 to about 3.0 mL of filtered sample-containing fluid in continuous streams through the aperture during one, two, three, or four compressions of the tube body. In one example, a total volume of about 2.5 to about 3.0 mL of filtered sample-containing fluid is propelled through the aperture during no more than two compressions of the tube body. Each compression may result in a continuous stream of a plurality of continuous streams of fluid being propelled through the aperture. Alternatively, the compressions of the tube body may result in a single continuous stream of fluid being propelled through the aperture.

Once filtered using the filter tube assembly 1100, the sample-containing fluid may be suitable for use in a molecular assay. For example, the filter tube assembly 1100 may be capable of filtering out certain molecules and/or particles capable of interfering with the molecular assay. Example inhibitors that can be removed by embodiments of filter tube assemblies described herein include proteins (blood and non-blood based), carbohydrates (for example mucin), immunoglobulins, cells and cellular debris, host microbiome, human genomic DNA (hugDNA), and salts. Advantageously, embodiments of filter tube assemblies according to the present disclosure can remove these and other inhibitors, thereby significantly improving assay sensitivity, decreasing time for DNA and RNA amplification, and in some cases preventing a failed test resulting from excessive inhibitors in a crude matrix.

Molecular assays may require relatively high volume of sample-containing fluid in comparison to other assay types (for example, immunoassays). Thus, it may be desirable that the filter tube assembly 1100 according to embodiments of the present disclosure can quickly and/or easily dispense a total volume as provided herein. The filter tube assembly 1100 can propel the filtered sample-containing fluid out of the dispense cap, such that filtration is relatively quick (for example, within seconds of or immediately in response to compression of the tube body 1104). In some examples, the filter tube assembly 1100 is capable of releasing the total volume of filtered sample-containing fluid in no more than two compressions of the tube body 1104. The compressions can be provided by a user compressing the tube body 1104. In some other examples, the filter tube assembly 1100 is capable of releasing the total volume of filtered sample-containing fluid in no more than one, two, three, or four compressions of the tube body 1104.

In some examples, the filter tube assembly 1100 can dispense a total volume of about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mL of filtered fluid, or any value or range within or bounded by any of these ranges or values, although values outside these values or ranges can be used in some cases. Additionally or alternatively, in some examples the filter tube assembly 1100 can dispense a total of about 2 to 4 mL of filtered fluid. Additionally or alternatively, in some examples the filter tube assembly 1100 can dispense a total of about 2.5 mL to about 3 mL of filtered fluid.

In some examples, upon a single compression of the tube body 1104, the filter tube assembly 1100 can dispense a total of about 0.4, 0.5, 0.6, 0.7, 0.8, 0.9, 1.0, 1.1, 1.2, 1.3, 1.4, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9,4.0, 4.1, 4.2, 4.3, 4.4, 4.5, 4.6, 4.7, 4.8, 4.9, or 5.0 mL of filtered fluid, or any value or range within or bounded by any of these ranges or values, although values outside these values or ranges can be used in some cases. Additionally or alternatively, in some examples the filter tube assembly 1100 can dispense a total of about 0.5 to 2 mL of filtered fluid per compression of the tube body 1104. Additionally or alternatively, in some examples the filter tube assembly 1100 can dispense a total of about 0.5 to 1.5 mL of filtered fluid per compression of the tube body 1104.

### 1. Dispense Cap

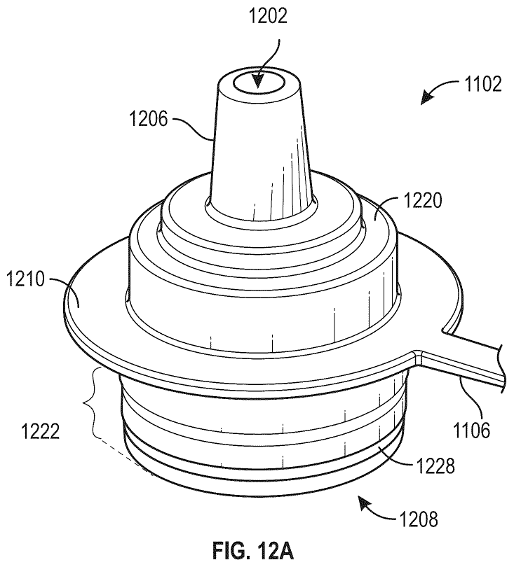

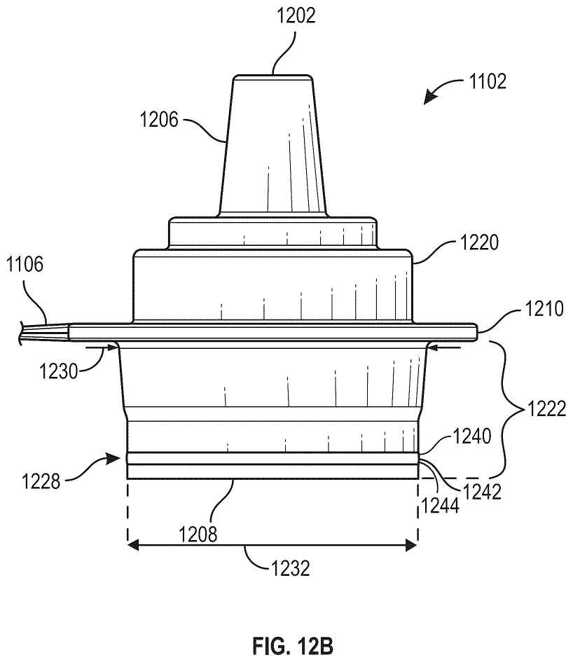

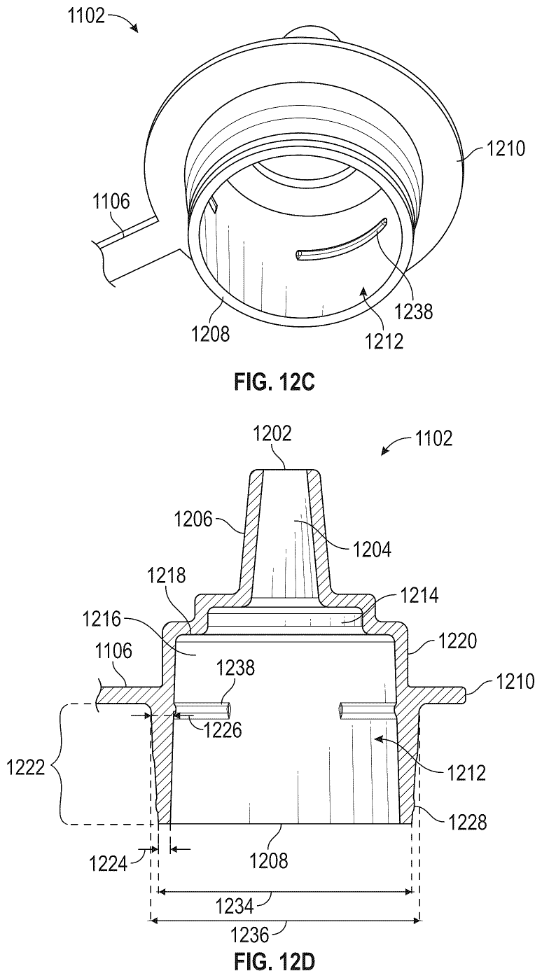

FIGS. 12A-12D illustrate views of the dispense cap 1102 included in the filter tube assembly 1100 according to an embodiment of the present disclosure. FIG. 12A illustrates an angled top view of the dispense cap 1102. FIG. 12B illustrates a side view of the dispense cap 1102. FIG. 12C illustrates an angled bottom view of the dispense cap 1102. FIG. 12D illustrates side cross-section of the dispense cap 1102.

The dispense cap 1102 can include an aperture 1202, a flow path 1204, a filter cavity 1216, an interior space 1212, a proximal end 1208, a nozzle 1206, a cap flange 1210, a cap body 1220, a collar 1222, and retention bumps 1238. The flow path 1204 can include a transition zone 1214.

The flow path 1204 may be a hollow space within the dispense cap 1102, for example within the nozzle 1206 of the dispense cap 1102. The aperture 1202 may be a hole at the distal end of the nozzle 1206. The flow path 1204 may be capable of allowing one or more propelled streams of sample-containing fluid (for example, filtered sample-containing fluid) to flow out the dispense cap 1102. In some examples, the diameter of the aperture 1202 may be about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mm, or any value or range within or bounded by any of these ranges or values, although values outside these values or ranges can be used in some cases. Additionally or alternatively, in some examples the aperture 1202 may have a diameter of about 2 to 4 mm. Additionally or alternatively, in some examples the aperture 1202 may have a diameter of about 2.8 to 3.2 mm. The filter cavity 1216 may be a space that can accept and hold a filter 1108 in accordance with embodiments of the present disclosure. The interior walls of the filter cavity 1216 can contact the filter 1108 when the filter 1108 is positioned within the dispense cap 1102. The filter 1108 may contact a distal surface 1218 of the filter cavity 1216 when positioned for use as part of the filter tube assembly 1100. The transition zone 1214 may be positioned proximate the filter cavity 1216. The diameter of the filter cavity 1216 may be about the same as or smaller than an outer diameter of the filter 1108.

The retention bumps 1238 may be positioned proximate the filter cavity 1216. The retention bumps 1238 may prevent or inhibit the filter 1108 from moving away from the filter cavity 1216. The retention bumps 1238 can act to secure the filter 1108 positioned within the filter cavity 1216, for example by contacting, engaging, or partially abutting a portion of the filter 1108. Alternatively, the retention bumps 1238 may be spaced apart from the filter 1108 when the filter 1108 sits within the filter cavity 1216 and abuts the distal surface 1218. In such examples, the retention bumps 1238 may contact the filter 1108 if it is moving away from the distal surface 1218. For particularly viscous sample fluids, it may be desirable to include the retention bumps 1238. The dispense cap 1102 may include one, two, three, four, five, six, or more retention bumps 1238. In some examples, the dispense cap 1102 can include three retention bumps 1238. The retention bumps 1238 may be positioned about a circumference of the interior space 1212 (for example, on an inner surface of the dispense cap 1102). In examples where there are two or more retention bumps, each of the retention bumps 1238 may be spaced apart from the other retention bumps 1238. Alternatively, the dispense cap 1102 may include a single retention bump 1238 that runs along a circumference of the interior space 1212. The retention bumps 1238 extend from an inner surface of the dispense cap 1102. The distance the retention bumps 1238 extend from the inner surface of the dispense cap 1102 may be 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 mm, or any value or range within or bounded by any of these ranges or values. Additionally or alternatively, the distance the retention bumps 1238 extend from the inner surface of the dispense cap 1102 may be about 0.15 to 0.25 mm. Additionally or alternatively, the distance the retention bumps 1238 extend from the inner surface of the dispense cap 1102 may be about 0.20 to 0.25 mm. Additionally or alternatively, the distance the retention bumps 1238 extend from the inner surface of the dispense cap 1102 may be about 0.21 to 0.23 mm. The retention bumps 1238 also allow the filter 1108 (particularly, the cup filter wall 1302) to flexibly pass over the retention bumps 1238 during assembly. It may be desirable that the retention bumps 1238 are shaped and sized such that the filter 1108 can pass over the retention bumps 1238 during assembly but the filter 1108 cannot pass back over the retention bumps 1238 during use of the filter tube assembly 1100. The retention bumps 1238 may be configured to retain the filter 1108 when a force is applied to the filter 1108 in a direction opposite to the direction of fluid flow out through the aperture 1202 during dispense (for example, a force applied to the filter 1108 between a first compression of the filter tube assembly 1100 and a second compression of the filter tube assembly 1100). In some instances, fluid and/or air may be sucked in an opposite direction of fluid flow through the filter tube assembly 1100 after a compression of the filter tube assembly 1100. The retention bumps 1238 may be configured to prevent or inhibit the filter 1108 from being dislodged even when the filter 1108 is subject to force of such fluid and/or air.

The transition zone 1214 can provide a volume where sample-containing fluid can be propelled out of the filter 1108. The transition zone 1214 has a diameter larger than the rest of the flow path 1204. Thus, because the transition zone 1214 does not greatly constrict the diameter of the flow path, the transition zone 1214 can thereby decrease back pressure (that is, a pressure within the tube body 1104 when, for example, the tube body 1104 is compressed) exerted on the filter 1108. By decreasing the pressure exerted on the filter 1108, the transition zone 1214 may reduce a risk of breakage, rupture, and/or slippage of the filter 1108 when the tube body 1104 is compressed. In some examples, the diameter of the transition zone 1214 can be about 4, 5, 6, 7, 8, 9, 10, 11, 12, 13, 14, 15, 16, 17, 18, 19, or 20 mm, or any value or range within or bounded by any of these ranges or values, although values outside these values or ranges can be used in some cases. Additionally or alternatively, in some examples the transition zone 1214 may have a diameter of about 8 to 12 mm. Additionally or alternatively, in some examples the transition zone 1214 may have a diameter of about 10 mm.

The nozzle 1206 may extend away from the cap body 1220. The interior surface of the nozzle 1206 may define a portion of the flow path 1204. The diameter of the flow path 1204 may decrease as the flow path 1204 extends towards the aperture 1202. In some embodiments, the dispense cap 1102 may include buttresses positioned about the nozzle 1206, which can support and/or stabilize the nozzle 1206. In some embodiments, the dispense cap 1102 may not include such buttresses.

The arm 1106 may extend from the cap flange 1210 to the tube body 1104. The arm 1106 may be able to bend and/or fold to allow the dispense cap 1102 to approach the open end of the tube body 1104. The material of the dispense cap 1102 and/or dimensions of the arm 1106 may affect the arm 1106′s capability of folding and/or bending. In some embodiments, the arm 1106 may include a notch that can allow the arm 1106 to bend and/or fold. As discussed, the dispense cap 1102, the arm 1106, and the tube body 1104 can be cast as a single component, for example as a single piece of plastic.

The cap body 1220 may include the interior space 1212. The cap body 1220 may include a collar 1222. The collar 1222 may be sized and shaped to engage the open end 1406 of the tube body 1104. An outer diameter of the collar 1222 may decrease as the collar 1222 extends from near the cap flange 1210 to the proximal end 1208. Such diameter decrease may allow for transmission of increasingly larger force by the collar 1222 on an interior surface of the tube body 1104 as the collar 1222 is further inserted into the interior of the tube body 1104 (for example, as the dispense cap 1102 is secured to the tube body 1104 before use of the filter tube assembly 1100 to filter sample-containing fluid). For example, a diameter 1230 may be equal to or larger than a diameter 1232. In some embodiments, the diameter 1230 may be about 10, 11, 12, 13, 14, 15, 16, 17, 18, 19, 20, 21, 22, 23, 24, or 25 mm, or any value or range within or bounded by any of these ranges or values, although values outside these values or ranges can be used in some cases. Additionally or alternatively, in some examples the diameter 1230 may be about 15 to 20 mm. Additionally or alternatively, in some examples the diameter 1230 may be about 16 to 19 mm. Additionally or alternatively, in some examples the diameter 1230 may be about 17 to 18 mm. In some embodiments, the diameter 1232 may be about 10, 11, 12, 13, 14, 15, 16, 17, 18, 19, 20, 21, 22, 23, 24, or 25 mm, or any value or range within or bounded by any of these ranges or values, although values outside these values or ranges can be used in some cases. Additionally or alternatively, in some examples the diameter 1232 may be about 14 to 19 mm. Additionally or alternatively, in some examples the diameter 1232 may be about 15 to 18 mm. Additionally or alternatively, in some examples the diameter 1232 may be about 16 to 17 mm. In some embodiments, the diameter 1232 may be smaller than the diameter 1230 by about 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm, or any value or range within or bounded by any of these ranges or values. Additionally or alternatively, the diameter 1232 may be smaller than the diameter 1230 by about 0.5 to 1.5 mm. Additionally or alternatively, the diameter 1232 may be smaller than the diameter 1230 by about 0.8 to 1.2 mm. Additionally or alternatively, the diameter 1232 may be smaller than the diameter 1230 by about 0.9 to 1.1 mm. Additionally or alternatively, the diameter 1232 may be smaller than the diameter 1230 by about 1.0 mm.

The collar 1222 may optionally include a ridge 1228. The ridge 1228 can engage features of the tube body 1104 in accordance with the present disclosure. The ridge 1228 may run at least a portion of the outer circumference of the collar 1222. With reference to FIG. 12B, the ridge 1228 is defined by an upper slope 1240, an apex 1242, and a lower slope 1244. In some examples, the angle of the lower slope 1244 on the ridge 1228 may be smaller than the angle of the upper slope 1240 on the ridge 1228. This difference in angle between the upper slope 1240 and lower slope 1244 may allow for a lower force needed when attaching the dispense cap 1102 to the tube body 1104 in comparison to the force needed to detach the dispense cap 1102 from the tube body 1104. The height of the apex 1242 may be chosen such that it is sufficiently small such as to not disrupt a plug seal between the dispense cap 1102 and the tube body 1104.

The thickness of the walls of the collar 1222 may decrease as the collar 1222 extends from the cap flange 1210 to the proximal end 1208 of the dispense cap 1102. For instance, a distal collar thickness 1226 may be equal to or larger than a proximal collar thickness 1224. In embodiments where the distal collar thickness 1226 is greater than the proximal collar thickness 1224, the collar 1222 may be capable of deflecting close to the proximal end 1208, which may allow the collar 1222 to fit within the open end of the tube body 1104. The taper on the collar 1222 may assist the user in creating an interference seal with the tube body 1104 having a gradually increasing force. In some embodiments, the gradually increasing force is configured to provide an ergonomic user experience, for example ensuring an efficient, reliable, and/or comfortable motion as the user couples the dispense cap 1102 to the tube body 1104. The proximal collar thickness 1224 can be about 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm, or any value or range within or bounded by any of these ranges or values, although values outside these values or ranges can be used in some cases. Additionally or alternatively, in some examples the proximal collar thickness 1224 may be about 0.5 to 1.0 mm. The distal collar thickness 1226 may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mm, or any value or range within or bounded by any of these ranges or values, although values outside these values or ranges can be used in some cases. Additionally or alternatively, in some examples the distal collar thickness 1226 may be about 2.5 to 3.5 mm. Additionally or alternatively, in some examples the distal collar thickness 1226 may be about 3.0 to 3.3 mm. Additionally or alternatively, in some examples the distal collar thickness 1226 may be about 3.1 to 3.2 mm.

In some embodiments, an outer diameter of the collar 1222 may decrease as the collar 1222 extends from the cap flange 1210. For instance, a distal collar diameter 1236 may be greater than a proximal collar diameter 1234. Such a decrease in the outer diameter of the collar 1222 may allow for to the dispense cap 1102 to be secured to the tube body 1104 via an interference fit. Such a decrease in the outer diameter of the collar 1222 may allow the dispense cap 1102 to be attached to the tube body 1104 with lower force than if there was no decrease in the outer diameter of the collar 1222. In some embodiments, the distal collar diameter 1236 is 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, or 21.0 mm or any value or range within or bounded by any of these ranges or values, although values outside these values or ranges can be used in some cases. Additionally or alternatively, in some examples the distal collar diameter 1236 may be about 16.5 to 18.5 mm. Additionally or alternatively, in some examples the distal collar diameter 1236 may be about 17.0 to 18.0 mm. In some embodiments, the proximal collar diameter 1234 is 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 mm or any value or range within or bounded by any of these ranges or values, although values outside these values or ranges can be used in some cases. Additionally or alternatively, in some examples the proximal collar diameter 1234 may be about 15.5 to 17.5 mm. Additionally or alternatively, in some examples the distal collar diameter 1236 may be about 16.0 to 17.0 mm. In some embodiments, a difference between the proximal collar diameter 1234 and the distal collar diameter 1236 is 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm, or any value or range within or bounded by any of these ranges or values, although values outside these values or ranges can be used in some cases. In some embodiments, the difference between the proximal collar diameter 1234 and the distal collar diameter 1236 is 0.5 to 1.5 mm. In some embodiments, the difference between the proximal collar diameter 1234 and the distal collar diameter 1236 is 1 mm.

In some embodiments, the dispense cap 1102 may include a plastic. In some embodiments, the plastic may be a polypropylene, a high-density polyethylene, or a linear low-density polyethylene (LLDPE). It may be desirable that the dimensions of the dispense cap 1102 and the material of the dispense cap 1102 are chosen such portions of the dispense cap 1102, for example the nozzle 1206, cap flange 1210, and/or the cap body 1220, do not substantially deform, or minimally deforms, when the tube body 1104 of the filter tube assembly 1100 is compressed. It may be desirable that the dimensions of the dispense cap 1102 and the material of the dispense cap 1102 are chosen such that portions of the dispense cap 1102, for example the collar 1222, can deform when the tube body 1104 of the filter tube assembly 1100 is compressed. In some examples, the Young's modulus of a material of the dispense cap 1102 may be about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 3000, 3010, 3020, 3030, 3040, 3050, 3060, 3070, 3080, 3090, or 4000 MPa, or any value or range within or bounded by any of these ranges or values, although values outside these values or ranges can be used in some cases. Additionally or alternatively, in some examples the Young's modulus of a material of the dispense cap 1102 may be between about 200 and 700 MPa. Additionally or alternatively, in some examples the Young's modulus of a material of the dispense cap 1102 may be between about 200 and 300 MPa. Additionally or alternatively, in some examples the Young's modulus of a material of the dispense cap 1102 may be between about 400 and 500 MPa.

The cap flange 1210 can abut a surface of the tube body 1104 in accordance with the present disclosure. In some embodiments, the cap flange 1210 need not abut the tube flange 1410 to confirm an effective seal is formed between the dispense cap 1102 and the tube body 1104. In such embodiments, there may be sufficient interference fit to form an effective seal between the dispense cap 1102 and the tube body 1104 without abutting the cap flange 1210 and the tube flange 1410.

### 2. Cup Filter

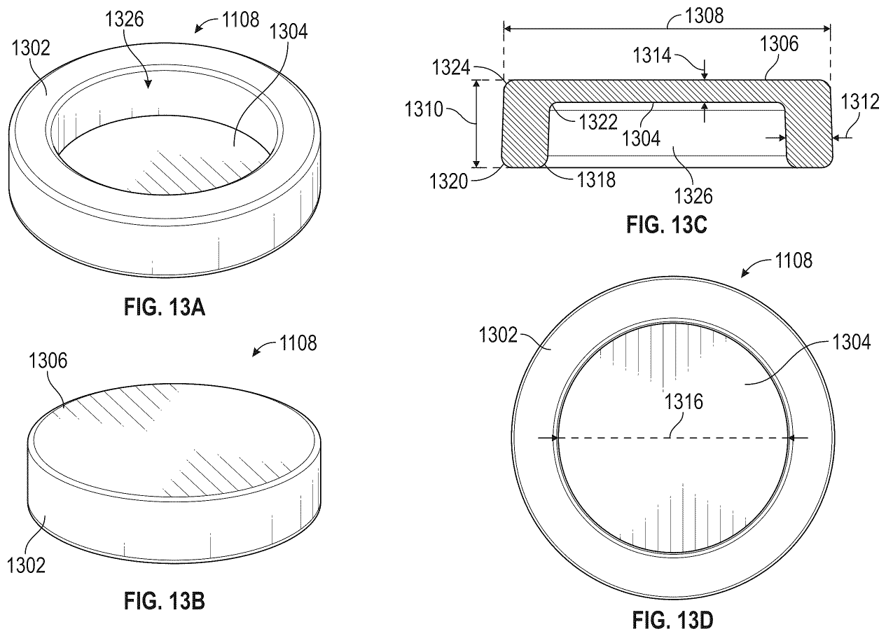

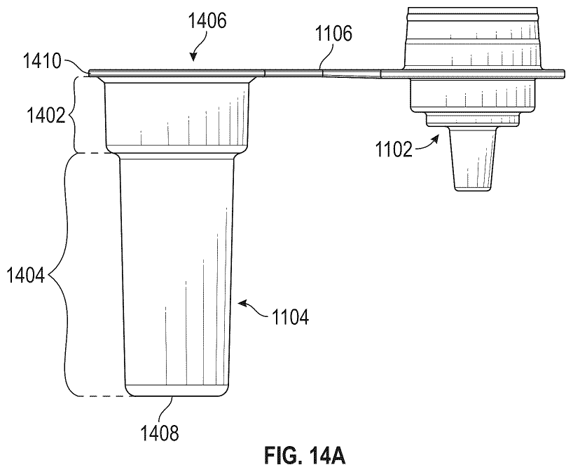

In some embodiments, the filter included in the filter tube assembly 1100 may be a cup filter. The cup filter 1108 is an example of a cup filter. As described above, the filter cavity 1216 may be a space that can accept and hold a filter 1108 may be received in the filter cavity 1216 of the dispense cap 1102. A cup filter may be cast as a single piece, for example as a single piece of material. A cup filter may also have a flat surface from which walls extend, the walls defining a concavity, such that the overall shape of the cup filter is similar to that of a cup. FIGS. 13A-13D illustrate views of the filter 1108. FIG. 13A illustrates an angled bottom view of the filter 1108. FIG. 13B illustrates an angled top view of the filter 1108. FIG. 13C illustrates a cross-sectional side view of the filter 1108. FIG. 13D illustrates a bottom elevation view of the filter 1108. The filter 1108 can include a cup filter wall 1302 having a filter height 1310 and a wall thickness 1312. The filter 1108 can also include a proximal surface 1304 and a distal surface 1306. The distal surface 1306 can have an outer diameter 1308. A distance between the distal surface 1306 and the proximal surface 1304 can define a filter depth 1314. The filter 1108 can also include an inner wall corner 1318, an outer wall corner 1320, a concavity corner 1322, and a distal corner 1324. The filter 1108 also includes a concavity 1326 having an inner diameter 1316. Each one of the inner wall corner 1318, the outer wall corner 1320, the concavity corner 1322, and the distal corner 1324 may be sharp, blunt, and/or rounded. It is to be understood that a cup filter may be used with any suitable filter tube assembly in accordance with the present disclosure.

In certain examples, the outer diameter 1308 of the filter 1108 may be approximately 8, 9, 10, 11, 12, 13, 14, 15, 16, 17, 18, 19, 20, 21, 22, 23, 24, 25, 26, 27, 28, 29, or 30 mm, or any value or range within or bounded by any of these ranges or values, although values outside these values or ranges can be used in some cases. Additionally or alternatively, in some examples the outer diameter 1308 may be about 12 to 16 mm. Additionally or alternatively, in some examples the outer diameter 1308 may be about 14 mm.

In certain examples, the surface area of the distal surface 1306 may be approximately 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mm² or any value or range within or bounded by any of these ranges or values, although values outside these values or ranges can be used in some cases. Additionally or alternatively, in some examples the area of the distal surface 1306 may be about 100 to 200 mm². Additionally or alternatively, in some examples the area of distal surface 1306 may be about 140 to 160 mm².

In certain examples, the inner diameter 1316 of the filter 1108 may be approximately 4, 5, 6, 7, 8, 9, 10, 11, 12, 13, 14, 15, 16, 17, 18, 19, 20, 21, 22, 23, 24, or 25 mm, or any value or range within or bounded by any of these ranges or values, although values outside these values or ranges can be used in some cases. Additionally or alternatively, in some examples the inner diameter 1316 may be about 8 to 12 mm. Additionally or alternatively, in some examples the inner diameter 1316 may be about 10 mm.

In certain examples, the surface area of the proximal surface 1304 may be approximately 30, 40, 50, 60, 70, 80, 90, 100, 110, 120, 130, 140, 150, 160, 170, 180, 190, or 200 mm² or any value or range within or bounded by any of these ranges or values, although values outside these values or ranges can be used in some cases. Additionally or alternatively, in some examples the area of the proximal surface 1304 may be about 50 to 100 mm². Additionally or alternatively, in some examples the area of the proximal surface 1304 may be about 70 to 90 mm².

In certain examples, the filter depth 1314 of the filter 1108 may be approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, or any value or range within or bounded by any of these ranges or values, although values outside these values or ranges can be used in some cases. Additionally or alternatively, in some examples the filter depth 1314 may be about 0.8 to 1.2 mm. Additionally or alternatively, in some examples the filter depth 1314 may be about 1 mm. Additionally or alternatively, in some examples the filter depth 1314 may be about 1.3 to 1.8 mm. Additionally or alternatively, in some examples the filter depth 1314 may be about 1.6 mm.

The cup filter wall 1302 may engage an inner circumference of the dispense cap 1102. Additionally or alternatively, the cup filter wall 1302 may engage a ridge of the inner circumference of the dispense cap 1102. The cup filter wall 1302 may be large enough (that is, the filter height 1310 may be long enough) such that the surface area of contact (and therefore friction) between the cup filter wall 1302 and a surface of the dispense cap 1102 within the interior space 1212 is sufficient to prevent and/or inhibit movement of the filter 1108 upon compression of the tube body 1104. In one example, the filter 1108 is held in position in the dispense cap 1102 based only on an interference fit. Advantageously, the ability to hold the filter 1108 in place in the dispense cap 1102 without adhesives or other chemical bonds can reduce or eliminate the introduction of confounding elements during filtering and dispensing of a sample. The cup filter wall 1302 may provide structural support to the portion of the filter 1108 between the proximal surface 1304 and the distal surface 1306 such that the portion of the filter 1108 between the proximal surface 1304 and the distal surface 1306 does not fold. In certain examples, the filter height 1310 of the filter 1108 may be approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 mm, or any value or range within or bounded by any of these ranges or values, although values outside these values can be used in some cases. Additionally or alternatively, in some examples the filter height 1310 may be about 3.6 mm to 4.0 mm. Additionally or alternatively, in some examples the filter height 1310 may be about 3.8 mm to 3.9 mm. The filter height 1310 may be several times larger than the filter depth 1314. In some examples, the filter height 1310 is 1.5×, 2×, 2.5×, 3×, 3.5×, 4×, 4.5×, 5×, 5.5×, 6×, 6.5×, 7×, 7.5×, 8×, 8.5×, 9×, 9.5×, or 10×, or any value or range within or bounded by any of these ranges or values, although values outside these values can be used in some cases. Additionally or alternatively, in some examples the filter height 1310 is about 1.5× to 3.5× larger than the filter depth 1314. Additionally or alternatively, in some examples the filter height 1310 is about 2.0× to 3.0× larger than the filter depth 1314. Additionally or alternatively, in some examples the filter height 1310 is about 2.3× to 2.6× larger than the filter depth 1314.

In certain examples, the wall thickness 1312 of the filter 1108 may be approximately 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, or any value or range within or bounded by any of these ranges or values, although values outside these values can be used in some cases. Additionally or alternatively, in some examples the wall thickness 1312 may be about 1.6 mm to 2.4 mm. Additionally or alternatively, in some examples the wall thickness 1312 may be about 1.8 mm to 2.2 mm. Additionally or alternatively, in some examples the wall thickness 1312 may be about 1.9 mm to 2.1 mm.

The filter 1108 includes a porous material. The porous material can include pores or spaces sized and shaped to allow fluid to pass from the proximal surface 1304 to the distal surface 1306, but through which some particles do not pass. The size of the pores can affect which particles are filtered as the sample-containing fluid is propelled through the filter 1108. It may be desirable that the size of the pores is small enough to filter particles that could interfere with a molecular assay. It may be desirable that the size of the pores is large enough to allow adequate volume and rate of fluid flow through the filter 1108 and/or lower a risk that the filter 1108 will become clogged with particles. The pore size (for example, average pore diameter) of the porous material may be 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μm or any value or range within or bounded by any of these ranges or values, although values outside these values or ranges can be used in some cases. Additionally or alternatively, in some examples the pore size may be about 5 to 25 μm. Additionally or alternatively, in some examples the pore size may be about 10 to 20 μm. Additionally or alternatively, in some examples the pore size may be about 50 to 120 μm. Additionally or alternatively, in some examples the pore size may be about 20 to 25 μm. Additionally or alternatively, in some examples the pore size may be about 5 to 15 μm. The porous material may include a plurality of tortuous paths. In some examples, the porous material may be hydrophobic. In some examples, the porous material may include a polyethylene (PE), a polypropylene (PP), or a polytetrafluoroethylene (PTFE). In some examples, the porous material may be hydrophilic. In some examples, the porous material may include a glass fiber or a melt blown polypropylene (PP). In some examples, the porous material may be a sintered porous material. In some examples, the porous material may be a sintered PE.

In one non-limiting embodiment, the filter 1108 includes a plurality of membranes, at least one membrane of the plurality of membranes having different properties (for example, different pore sizes and/or different hydrophobic/hydrophilic properties). The plurality of membranes can be arranged in a stack. One membrane can be arranged closer to the proximal surface 1304 and serve as a prefilter. The membrane serving as a prefilter can include pores of a size and/or shape to retain large particles, while one or more downstream membranes can include pores of a size and/or shape to retain smaller particles. In one non-limiting example, a first tube assembly includes a filter including a single membrane, configured to dispense a first sample-containing fluid to be tested for a first analyte of interest. A second tube assembly includes a filter including two membranes, configured to dispense a second sample-containing fluid to be tested for a second analyte of interest different than the first analyte of interest. The second sample-containing fluid can include a crude matrix for which a two-level filtering format is optimal, relative to the first sample-containing fluid.

### 3. Tube Body

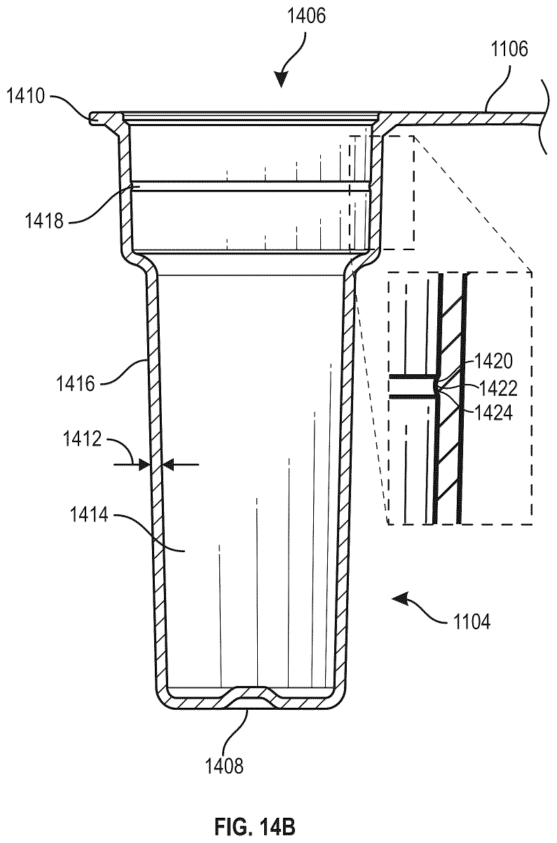

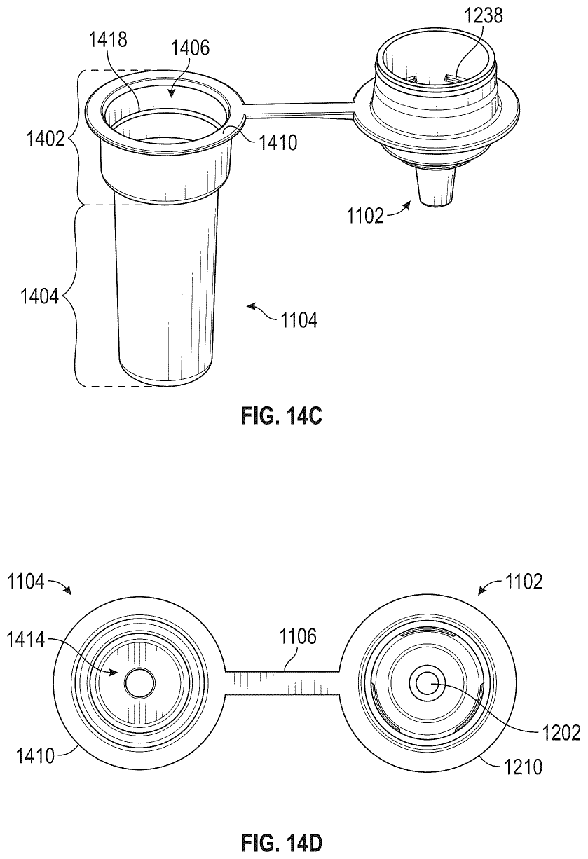

FIGS. 14A-14D illustrate views of the tube body 1104 included in the filter tube assembly 1100 according to an embodiment of the present disclosure. Also illustrated are the dispense cap 1102 and the arm 1106. FIG. 14A illustrates a side view of the tube body 1104. FIG. 14B illustrates a cross-sectional side view of the tube body 1104. FIG. 14C illustrates an angled view of the tube body 1104. FIG. 14D illustrates a top-down view of the tube body 1104. The tube body 1104 can include an open end 1406, a closed end 1408, a tube flange 1410, a tube wall 1416 having a thickness 1412, and an interior volume 1414. The tube body 1104 can optionally include a base region 1404 and a neck region 1402 including a ridge 1418.

The interior volume 1414 can hold a fluid, such as a sample-containing fluid. In some examples, the fluid is a buffer to which a sample is added, forming a sample-containing fluid. The dispense cap 1102 can be positioned over the open end 1406 of the tube body 1104, such that the interior space 1212 of the dispense cap 1102 is fluidically connected to the interior volume 1414, thereby allowing the filter 1108 to be contacted by the sample-containing fluid if the tube body 1104 is compressed and/or the filter tube assembly 1100 is inverted. In some examples, the interior volume 1414, in an uncompressed state, is approximately 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11, 11.5, 12, 12.5, 13, 13.5, 14, 14.5, or 15 mL or any value or range within or bounded by any of these ranges or values, although values outside these values or ranges can be used in some cases. Additionally or alternatively, in some examples the interior volume 1414 is about 4 to 6 mL in an uncompressed state. Additionally or alternatively, in some examples the interior volume 1414 is about 5 mL. Additionally or alternatively, in some examples the interior volume 1414 is about 6.7 mL. In some examples, the tube body 1104 can hold approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11, 11.5, 12, 12.5, 13, 13.5, 14, 14.5, or 15 mL of sample-containing fluid or any value or range within or bounded by any of these ranges or values, although values outside these values or ranges can be used in some cases. In one non-limiting embodiment, the tube body 1104 holds about 6.7 mL of sample-containing fluid. Additionally or alternatively, in some examples the tube body 1104 can hold about 2 to 4 mL of sample-containing fluid. Additionally or alternatively, in some examples the tube body 1104 can hold about 3 mL of sample-containing fluid. In some examples, the tube body 1104 can include approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mL of headspace volume (for example, a volume within the tube body 1104 that is not occupied by fluid when the buffer or sample-containing fluid is present within the tube body 1104) or any value or range within or bounded by any of these ranges or values, although values outside these values or ranges can be used in some cases. Additionally or alternatively, in some examples the tube body 1104 can include about 1 to 3 mL of headspace volume. Additionally or alternatively, in some examples the tube body 1104 can include about 2 mL of headspace volume.

In some embodiments, the interior volume 1414 of the tube body 1104 can be reduced in order to propel the sample-containing fluid from the filter tube assembly 1100 to a test device, for example for performing a molecular assay, such as but not limited to nucleic acid amplification. Reduction of the interior volume 1414 can be accomplished in several ways. In the embodiment illustrated in FIGS. 14A-14D, the material of the tube body 1104 is flexible enough, in particular the base region 1404, to allow the user to compress the walls 1416 of the tube body 1104 to propel one or more streams of filtered sample-containing fluid to the test device through the aperture 1202 of the dispense cap 1102. The one or more streams can be continuous streams of fluid propelled through the aperture 1202. The flexibility can arise from a combination of thickness of the walls 1416 and modulus (for example, a Young's modulus) of the material included in the tube body 1104. This combination of thickness of walls 1416 and material of the tube body 1104 can be chosen so that the tube body 1104 is compressible by a user. In a second embodiment, the tube body 1104 can have thin sections in the walls 1416, running axially and/or radially, that give the tube body 1104 hinge points where the walls 1416 can flex while other portions of the walls 1416 are thicker and/or stiffer. The user can then compress the tube body 1104, which flexes at the thin hinge points thus reducing the interior volume 1414 and propelling the sample-containing fluid out through the filter 1108 and flow path 1204 and out the aperture 1202 of the dispense cap 1102, without the entire wall 1416 being thin enough to flex. Other approaches to propel a stream or other volume of sample-containing fluid from the tube body 1104 through the dispense cap 1102 are possible. In other embodiments, the tube body 1104 may not require compression or squeezing to dispense fluid from the filter tube assembly 1100 and may dispense drops of fluid upon inversion of the filter tube assembly 1100.

In some embodiments, the thickness of the material of the tube body 1104 at the tube flange 1410 may be larger than or equal to that of the thickness 1412 of the walls 1416. In some embodiments, the thickness 1412 of the material of the closed end 1408 may be equal to or larger than the thickness 1412 of the walls 1416. In some examples, the thickness 1412 of the walls 1416 may be about 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, or 1.25 mm or any value or range within or bounded by any of these ranges or values, although values outside these values or ranges can be used in some cases. Additionally or alternatively, in some examples the thickness 1412 is about 0.2 to 1.0 mm. Additionally or alternatively, in some examples the thickness 1412 is about 0.50 to 1.0 mm. Additionally or alternatively, in some examples the thickness 1412 is about 0.60 to 0.90 mm. Additionally or alternatively, in some examples the thickness 1412 is about 0.70 to 0.80 mm. Additionally or alternatively, in some examples the thickness 1412 is about 0.75 mm.

The neck region 1402 and the base region 1404 allow for variation in thickness of the tube body 1104. As an illustrative example, the base region 1404 may be sized and shaped such that the height of any liquid contained within the tube body 1104 reaches a suitable height such that a swab head can be submerged within the liquid. As an illustrative example, the base region 1404 may have an inner diameter of 12-13 mm and a height of 28-29 mm, while the neck region 1402 may have an inner diameter of 16-17 mm and a height of 7-8 mm. In such an example, the tube body 1104 may hold 2 mL buffer and may allow for an industry-standard sample collection swab to be fully submerged when received by the tube body 1104. As another illustrative example, the base region 1404 may be sized and shaped such that the tube body 1104 can be placed and held within a standard tube rack. The neck region 1402 may be sized and shaped to allow for a dispense cap 1102 sufficiently large to allow a suitable filter surface area (for example, a filter surface area of filter 1108).

The ridge 1418 may be positioned within the neck region 1402 as shown in FIGS. 14B and 14C. With reference to FIG. 14B, the ridge 1418 is defined by an upper slope 1420, an apex 1422, and a lower slope 1424. In some examples the angle of the upper slope 1420 on the ridge 1418 may be smaller than the angle of the lower slope 1424 on the ridge 1418. This difference in angle between the upper slope 1420 and lower slope 1424 may allow for a lower force needed when attaching the dispense cap 1102 to the tube body 1104 in comparison to the force needed to detach the dispense cap 1102 from the tube body 1104. The height of the apex 1422 may be chosen such that it is sufficiently small such as to not disrupt a plug seal between the dispense cap 1102 and the tube body 1104.

In some embodiments, the tube body 1104 may include a plastic. In some embodiments, the plastic may be a polyethylene. In some embodiments, the plastic may be a low-density polyethylene (LDPE). In some embodiments, the plastic may be a linear low-density polyethylene (LLDPE). A tube body 1104 including an LLDPE may advantageously not experience cracking after multiple squeezes. A tube body 1104 including LLDPE may advantageously allow for a relatively low squeeze force needed to compress the filter tube assembly 1100 while simultaneously allowing for the wall thickness 1412 to be adequately thick to manufacture. A tube body 1104 including an LLDPE may advantageously exert a higher force of interference fit on the dispense cap 1102, even while requiring similar or lower force from a user to secure the dispense cap 1102 to the tube body 1104. In turn, greater force of interference fit may be advantageous because the greater force of interference fit can allow a more robust seal to be formed. Embodiments of tube bodies including an LLDPE and having a greater force of interference fit according to the present disclosure can advantageously provide more robust tube assemblies that are not as sensitive to non-circularity, molding tolerances, or damage in the seal area. In some examples, the Young's modulus of a material of the tube body 1104 may be about 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 MPa, or any value or range within or bounded by any of these ranges or values, although values outside these values or ranges can be used in some cases. Additionally or alternatively, in some examples the Young's modulus of a material of the tube body 1104 may be between about 200 and 700 MPa. Additionally or alternatively, in some examples the Young's modulus of a material of the tube body 1104 may be between about 200 and 300 MPa. Additionally or alternatively, in some examples the Young's modulus of a material of the tube body 1104 may be between about 400 and 500 MPa.

The material included in the tube body 1104 may be optically transparent, for example transparent to visible light. In some examples, the transparency of the material of the tube body 1104 may allow a user to view the volume of sample-containing fluid and/or buffer held within the interior volume 1414. In some embodiments, a fill line may be included that can indicate to a user whether there is an adequate amount of sample-containing fluid and/or buffer within the interior volume 1414 of the tube body 1104. In some embodiments, the fill line can be a raised feature molded into the walls 1416. In some embodiments, the fill line can be a depression molded into the walls 1416. In some embodiments, the fill line can be printed on the walls 1416, for example with an ink or other marking material. In some embodiments, the tube body 1104 can include more than one fill line. In such embodiments, each fill line may indicate a different volume. When the filter tube assembly 1100 is oriented such that the open end 1406 is pointed upward and the closed end 1408 of the tube body 1104 is pointed downward, the user may be able to compare the vertical position of the upper surface of the buffer and/or sample-containing fluid within the interior volume 1414 relative to the position of the fill line. In some examples, the fill line can indicate whether there is a minimum required volume of sample-containing fluid present to perform a molecular assay. In some examples where a buffer fluid is stored within the interior volume 1414 over a period of days, weeks, months, and/or years, the fill line can indicate whether buffer volume has been lost beyond a threshold indicated by the fill line, for example due to evaporation, leakage, and/or escape from the tube body 1104.

The dispense cap 1102 may be positioned on and/or over the open end 1406. The collar 1222 of the dispense cap 1102 can engage an interior of the tube body 1104, for example the interior of the neck region 1402. When the dispense cap 1102 is secured to the open end 1406 of the tube body 1104, the ridge 1228 of the collar 1222 can be configured to engage with the ridge 1418 of the neck region 1402. Engagement of the ridge 1228 with the ridge 1418 can inhibit and/or prevent removal of the dispense cap 1102 from the open end 1406 of the tube body 1104. A seal between the collar 1222 and the neck region 1402 may ensure that none or substantially none of the sample fluid leaks from the filter tube assembly 1100. A seal between the collar 1222 and the neck region 1402 may ensure that maximal fluid is directed toward the filter 1108 when the filter tube assembly 1100 is inverted and/or compressed. Securing the dispense cap 1102 in the neck region 1402 of the tube body 1104 initially involves applying sufficient force to the top of the dispense cap 1102 to force the collar 1222 into the open end 1406 of the tube body 1104. As continued pressure is applied to the dispense cap 1102, the ridge 1228 slides over the ridge 1418. The ridge 1228 and the ridge 1418 may slide over each other such that the user has tactile and/or auditory confirmation that the ridges 1228 and 1418 have engaged and that there is a seal formed between the dispense cap 1102 and the open end 1406 of the tube body 1104 (for example, a plug seal at the assembly portion 1710 discussed with reference to FIG. 17). The tactile feedback may be particularly helpful, as the sound of the ridges 1228 and 1418 sliding past each other may be dampened by virtue of the sound being made within the interior of the tube assembly 1100. The neck portion 1402 can be shaped and sized to reduce deformation, such as deformation into a non-circular cross-section, of the neck portion 1402 when the base region 1404 is compressed during fluid dispense. Reducing non-circularity of the neck portion 1402 can advantageously aid in maintaining the plug seal in the assembly portion 1710 during fluid dispense.

When the dispense cap 1102 is positioned on and/or over the open end 1406, the cap flange 1210 of the dispense cap 1102 may abut the tube flange 1410. The contact of the cap flange 1210 and the tube flange 1410 may thereby prevent further translation of the dispense cap 1102 in the direction of the closed end 1408 of the tube body 1104. The contact of the cap flange 1210 may provide a tactile response to a user securing the dispense cap 1102 to the closed end 1408 of the tube body 1104.

### 4. Seal

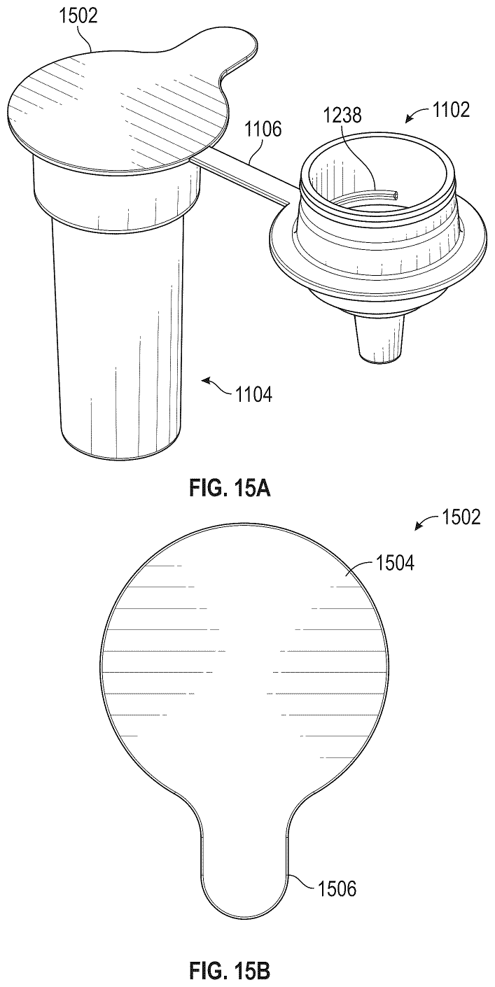

In some embodiments, particularly those where buffer fluid and/or reagents are stored within the tube body 1104 for an extended period (for example, for days, weeks, months, and/or years) before the filter tube assembly 1100 is used to filter a sample-containing fluid, a seal may be affixed to the open end of the tube body 1104. FIGS. 15A-15B illustrate such a seal. FIG. 15A illustrates the filter tube assembly 1100 having a seal 1502. FIG. 15B illustrates a top-down view of the seal 1502. The seal 1502 can cover the open end 1406 of the tube body 1104 as illustrated in FIG. 15A, thereby preventing evaporation, contamination, or the like of any buffer fluid and/or reagents contained within the tube body 1104. The seal 1502 can create a fluid-tight barrier between the interior volume 1414 of the tube body 1104 and the exterior. As an illustrative example, the seal 1502 may be a foil heat-pressed seal. The seal 1502 may be heat-pressed to the tube flange 1410 to create a seal. Alternatively, induction sealing may be used to bond the seal 1502 to the tube flange 1410. Other bonding methods can be suitably implemented. For example, the seal 1502 may be bonded to the tube flange 1410 using an adhesive. The seal 1502 can include a covering portion 1504 and a tab portion 1506. The covering portion 1504 can cover the open end 1406 of the tube body 1104. The tab portion 1506 can be grasped by a user and pulled, allowing for removal of the seal 1502 from the tube body 1104. Advantageously, the seal 1502 can be removed from the tube body 1104 while minimizing risk of user contact with or contamination of any buffer fluid and/or reagents contained within the tube body 1104.

### 5. Assembly

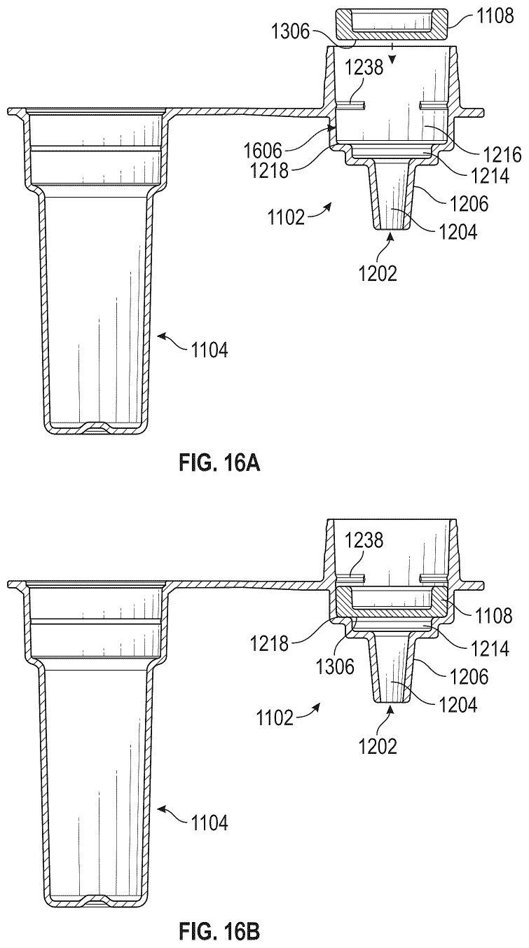

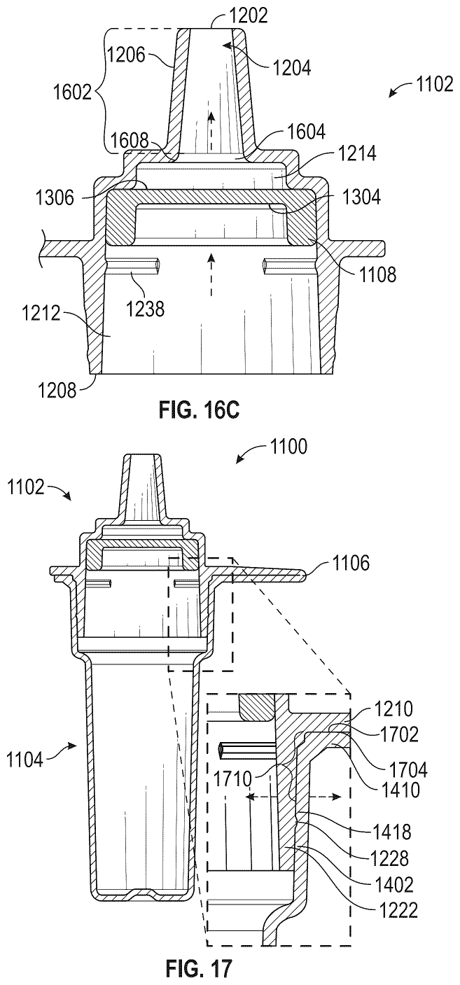

FIGS. 16A-17 illustrate cross-sectional views of the filter tube assembly 1100, showing interaction of the dispense cap 1102, the tube body 1104, and the filter 1108. FIG. 16A illustrates a cross-sectional side view of the filter tube assembly 1100 showing insertion of the filter 1108 to the dispense cap 1102. FIG. 16B illustrates a cross-sectional side view of the filter tube assembly 1100 showing the filter 1108 positioned within the dispense cap 1102. FIG. 16C illustrates a cross-sectional side view of the dispense cap 1102 with the filter 1108 positioned inside and showing the path of fluid flow through the dispense cap 1102. FIG. 17 illustrates a cross-sectional side view of the filter tube assembly 1100 where the dispense cap 1102 has been attached to the open end 1406 of the tube body 1104 and includes an inset showing the interaction of the collar 1222 and the neck region 1402.

As illustrated in FIGS. 16A and 16B, the filter 1108 can be positioned within the dispense cap 1102 such that the distal surface 1306 of the filter 1108 abuts a distal surface 1218 of the dispense cap 1102. The distal surface 1218 can contact the distal surface 1306, thereby preventing further motion of the filter 1108 towards the aperture 1202 of the dispense cap 1102. The outer diameter 1308 of the filter 1108 may be about equal to or slightly larger than a diameter of the filter cavity 1216 of the dispense cap 1102. Accordingly, the filter 1108 may fit snugly within the filter cavity 1216 when positioned within the dispense cap 1102. For example, the filter 1108 may engage a section 1606 of the interior wall of the dispense cap 1102 with an interference fit. The fit of the filter 1108 may substantially prevent and/or inhibit motion of the filter 1108 within the dispense cap 1102 when a user compresses the tube body 1104 to drive sample-containing fluid through the filter 1108 and out of the dispense cap 1102.

The dashed arrows in FIG. 16C indicate the direction (for example, in the distal direction, away from the proximal end 1208 of the dispense cap 1102) of fluid flow through the dispense cap 1102 when the tube body 1104 of the filter tube assembly 1100 is compressed. When the tube body 1104 of the filter tube assembly 1100 is compressed, the sample-containing fluid can flow from the interior space 1212 and/or interior volume 1414 (which are fluidically connected) through the filter 1108, through the flow path 1204 (which includes the transition zone 1214, an intermediate portion 1604, and a distal portion 1602), and out the aperture 1202.

As previously discussed, the transition zone 1214 can reduce back pressure (that is, high pressure in the interior space 1212 relative to that of the flow path 1204) acting on the filter 1108. The transition zone 1214 provides a space with a diameter not much smaller than that of the outer diameter 1308 of filter 1108. In some examples, the outer diameter 1308 of the filter 1108 is about 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or 2.00 times larger than the diameter of the transition zone 1214, or any value or range within or bounded by any of these ranges or values, although values outside these values or ranges can be used in some cases. Additionally or alternatively, in some examples the outer diameter 1308 of the filter 1108 is about 1.2-1.6 times larger than the diameter of the transition zone 1214. Additionally or alternatively, in some examples the outer diameter 1308 of the filter 1108 is about 1.3-1.5 times larger than the diameter of the transition zone 1214. Additionally or alternatively, in some examples the outer diameter 1308 of the filter 1108 is about 1.35-1.45 times larger than the diameter of the transition zone 1214. Additionally or alternatively, in some examples the diameter of the filter 1108 is about 1.4 times larger than the diameter of the transition zone 1214.

The shape and/or dimensions of the distal portion 1602 and the intermediate portion 1604 of the flow path 1204 may also affect pressure on the filter 1108. It may be desirable that the interior surface of the dispense cap 1102 defining the intermediate portion 1604, referred to as curved portion 1608, include a gradual curve such that the diameter of the flow path 1204 does not suddenly decrease from the transition zone 1214 to the distal portion 1602. A gradual change in diameter of the flow path 1204 may ensure that there are no sharp increases or decreases in pressure of the sample-containing fluid. The interior walls of the dispense cap 1102 defining the distal portion 1602 may be shallowly sloped. In some embodiments, a diameter at a proximal portion of the distal portion 1602 (for example, the portion proximate to the intermediate portion 1604) is larger than a diameter of the aperture 1202.

The ratio of the surface area of the filter 1108 to the size of the interior volume 1414 may affect the maximum flow rate of the sample-containing fluid out of the filter tube assembly 1100 upon compression of the tube body 1104. It may be desirable that the maximum flow rate is high enough such that the filter tube assembly 1100 can dispense the total volume of filtered sample-containing fluid for the molecular assay within no more than one, two, three, or four compressions of the tube body 1104 as previously discussed. In some examples, the ratio a:v of the surface area of the distal surface 1306 of the filter 1108 to the interior volume 1414 of the tube body 1104 may be about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 m⁻¹, or any value or range within or bounded by any of these ranges or values, although values outside these values or ranges can be used in some cases. Additionally or alternatively, in some examples the ratio a:v of the surface area of the distal surface 1306 of the filter 1108 to the interior volume 1414 of the tube body 1104 is about 20-40 m⁻¹. Additionally or alternatively, in some examples the ratio a:v of the surface area of the distal surface 1306 of the filter 1108 to the interior volume 1414 of the tube body 1104 is about 25-35 m⁻¹. Additionally or alternatively, in some examples the ratio a:v of the surface area of the distal surface 1306 of the filter 1108 to the interior volume 1414 of the tube body 1104 is about 30 m⁻¹. The maximum flow rate may be affected by the inner diameter 1316 of the filter 1108. Fluid may pass through the surface area of the proximal surface 1304 of the filter 1108 at a higher flow rate than through the cup filter wall 1302, due to the difference in flow path through the filter height 1310 and the filter depth 1314. The portion of the distal surface 1218 that contacts the filter 1108 may impede flow through the contacted portion of the filter 1108. This is all to say that, in some embodiments, the surface area of the filter 1108 available for fluid flow may be less than the surface area of distal surface 1306. In some examples, the ratio a:v of the proximal surface 1304 of the filter 1108 to the interior volume 1414 of the tube body 1104 may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 m⁻¹, or any value or range within or bounded by any of these ranges or values, although values outside these values or ranges can be used in some cases. Additionally or alternatively, in some examples the ratio a:v of the proximal surface 1304 of the filter 1108 to the interior volume 1414 of the tube body 1104 is about 10-25 m⁻¹. Additionally or alternatively, in some examples the ratio av of the proximal surface 1304 of the filter 1108 to the interior volume 1414 of the tube body 1104 is about 15-20 m⁻¹. Additionally or alternatively, in some examples the ratio a:v of the proximal surface 1304 of the filter 1108 to the interior volume 1414 of the tube body 1104 is about 17 m⁻¹.

FIG. 17 illustrates a cross-sectional view of the filter tube assembly 1100 according to an embodiment of the present disclosure where the dispense cap 1102 is attached to the tube body 1104. When the dispense cap 1102 is attached to the open end 1406 of the tube body 1104, the collar 1222 can engage with the neck region 1402 to create a fluid-tight barrier. Engagement between the collar 1222 and the neck region 1402 can result in transmission of lateral forces as indicated by the dashed arrows. Engagement between the collar 1222 and the neck region 1402 can be an interference fit. Such an interference fit may occur at an assembly portion 1710. The interference fit at the assembly portion 1710 may form a plug seal. The force exerted by such an interference fit can depend on the material of the tube body 1104 and the material of the dispense cap 1102. The force exerted by such an interference fit can depend on the shaping and/or sizing of the tube body 1104 and the dispense cap 1102. The proximal collar thickness 1224 being smaller than the distal collar thickness 1226 allows for greater deflection of the collar 1222 the further the collar 1222 extends from cap flange 1210. The collar 1222 may be shaped and sized such that a user is able to attach the dispense cap 1102 to the tube body 1104 and the engagement of the collar 1222 with the neck region 1402 is still sufficient to prevent and/or inhibit the dispense cap 1102 from coming off the open end 1406 of the tube body 1104 upon compression of the tube body 1104 by a user. The ridge 1228 of the dispense cap 1102 and the ridge 1418 of the tube body 1104 can engage. Engagement of the ridges 1228 and 1418 can prevent and/or inhibit the dispense cap 1102 from coming detached from the open end 1406 of the tube body 1104. When the dispense cap 1102 is attached to the tube body 1104, a proximal surface 1702 of the cap flange 1210 of the dispense cap 1102 can abut a distal surface 1704 the tube flange 1410 of the tube body 1104. Abutment of the proximal surface 1702 of the cap flange 1210 with the distal surface 1704 the tube flange 1410 can prevent and/or inhibit motion of the dispense cap 1102 towards the closed end 1408 of the tube body 1104. As discussed, in some embodiments, the cap flange 1210 need not abut the tube flange 1410 to confirm an effective seal is formed between the dispense cap 1102 and the tube body 1104. In such embodiments, there may be sufficient interference fit to form an effective seal between the dispense cap 1102 and the tube body 1104 without abutting the cap flange 1210 and the tube flange 1410.

The shape, taper, and/or length of collar 1222 can affect the force to remove the dispense cap 1102 from the tube body 1104. Taper, as discussed herein with reference to the collar 1222, can refer to a reduction in thickness of the collar 1222 as the collar 1222 extends away from the cap flange 1210 (that is, with reference to FIG. 12D, the proximal collar thickness 1224 being smaller than the distal collar thickness 1226). Taper, as discussed herein with reference to the collar 1222, can additionally or alternatively refer to a reduction in outer diameter of the collar 1222 as the collar 1222 extends away from the cap flange 1210 (that is, with reference to FIG. 12D, the proximal collar diameter 1234 may be smaller than the distal collar diameter 1236). For instance, reducing taper and/or increasing length of the collar 1222 may, for example, increase the force to remove the dispense cap 1102 from the tube body 1104. The taper on the collar 1222, paired with a shape of the neck region 1402, may assist the user in creating an interference seal with the tube body 1104 having a gradually increasing force. In some embodiments, the gradually increasing force is configured to provide an ergonomic user experience, for example ensuring an efficient, reliable, and/or comfortable motion as the user couples the dispense cap 1102 to the tube body 1104.

The shape of the ridges 1228 and 1418 can each affect the amount of force to attach the dispense cap 1102 to the tube body 1104 and/or remove the dispense cap 1102 from the tube body 1104. For instance, the shape and/or size of the ridges 1228 and 1418 can each be altered to increase and/or decrease the force to attach the dispense cap 1102 to the tube body 1104 and/or remove the dispense cap 1102 from the tube body 1104. When either or both of the ridges 1228 and 1418 is relatively small (that is, not extending far from an outer surface of the collar 1222 or from an inner surface of the neck region 1402, respectively), the force needed to attach the dispense cap 1102 onto the tube body 1104 may be relatively low. Increasing the size (that is, the distance the ridge 1228 extends from the outer surface of the collar 1222 or the distance or the distance the ridge 1418 extends from an inner surface of the neck region 1402) of either or both of ridges 1228 and 1418 may increase the force to attach the dispense cap 1102 to the tube body 1104. In such embodiments, the sliding of the ridge 1228 past the ridge 1418 may provide a user with tactile or auditory feedback that the dispense cap 1102 is secured to the tube body 1104 and/or that a seal has been established.

In some examples, the force needed to attach the dispense cap 1102 to the tube body 1104 is no more than about 10, 20, 30, 40, 50, 60, 70, 80, 90, 100, 110, 120, 130, 140, or 150 N, or any value or range within or bounded by any of these ranges or values, although values outside these values or ranges can be used in some instances. In some examples, the force to attach the dispense cap 1102 to the tube body 1104 is from about 20 to 100 N. In some examples, the force to attach the dispense cap 1102 to the tube body 1104 is no more than about 60 N. In some examples, the force exerted to attach the dispense cap 1102 to the tube body 1104 may be less than the force exerted to remove the dispense cap 1102 from the tube body 1104. In some examples, the force exerted to remove the dispense cap 1102 from the tube body 1104 can be about 10, 15, 20, 25, 30, 35, 40, 45, 50, 55, 60, 65, 70, 75, 80, 85, 90, 95, 100, 105, 110, 115, 120, 125, 130, 135, 140, 145, or 150 N, or any value or range within or bounded by any of these ranges or values, although values outside these values or ranges can be used in some cases. Additionally or alternatively, in some examples the force exerted to remove the dispense cap 1102 from the tube body 1104 can be about 10 to 50 N. Additionally or alternatively, in some examples the force exerted to remove the dispense cap 1102 from the tube body 1104 can be about 20 to 40 N. Additionally or alternatively, in some examples the force exerted to remove the dispense cap 1102 from the tube body 1104 can be about 25 to 35 N. Additionally or alternatively, in some examples the force exerted to remove the dispense cap 1102 from the tube body 1104 can be about 30 N. It may be desirable that the forces to attach the dispense cap 1102 to the tube body 1104 and to remove the dispense cap 1102 from the tube body 1104 are sufficiently low such that a user can attach and/or remove the dispense cap 1102 manually. In some embodiments, the force to attach the dispense cap 1102 to the tube body 1104 is such that a user can attach the dispense cap 1102 to the tube body 1104 using one hand. It may be desirable that the force to remove the dispense cap 1102 from the tube body tube body 1104 is sufficiently high such that the dispense cap 1102 is not ejected during normal use of the filter tube assembly 1100.

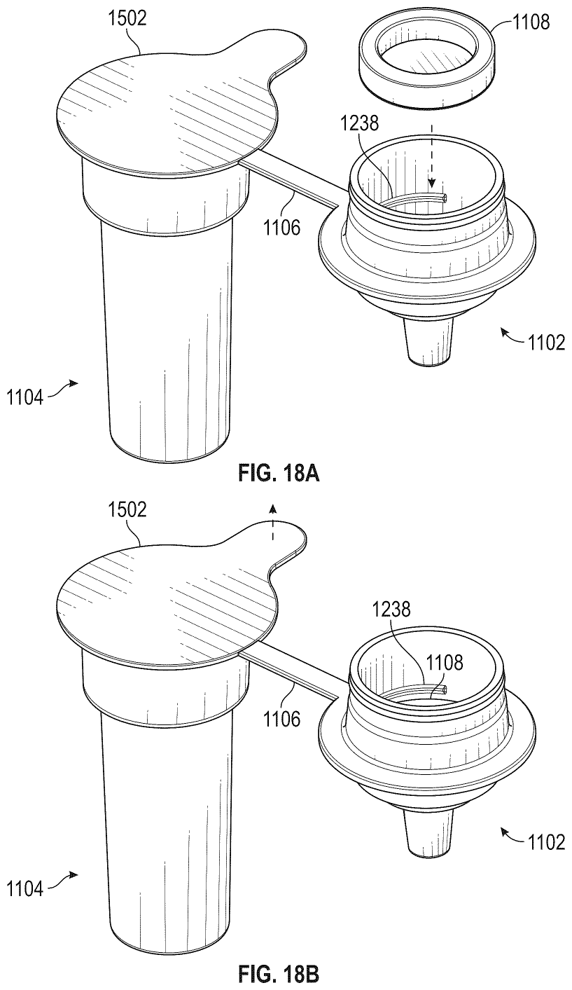

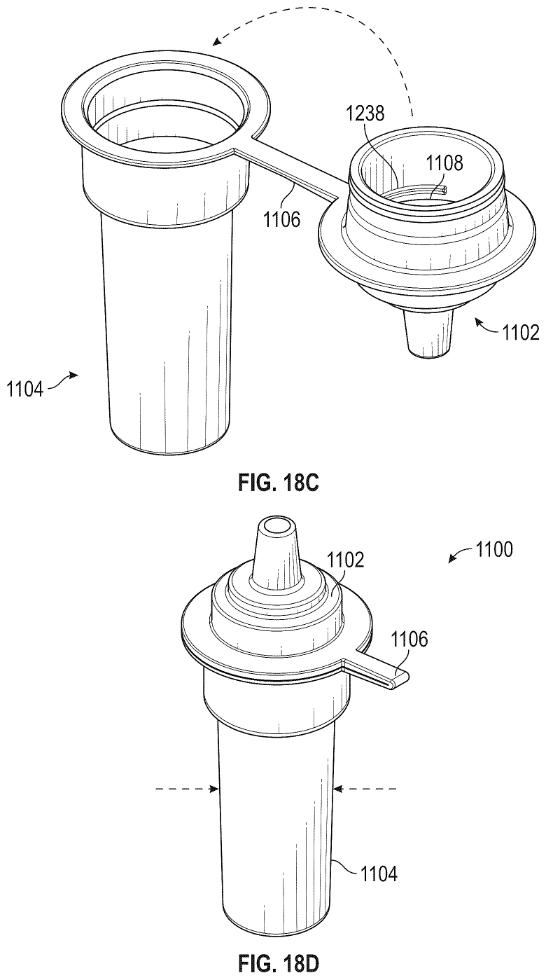

FIGS. 18A-18D illustrate a series of steps for preparing the filter tube assembly 1100 for use in filtering a sample-containing fluid. FIG. 18A illustrates insertion of the filter 1108 into the dispense cap 1102. FIG. 18B illustrates removal of the seal 1502 from the tube body 1104. FIG. 18C illustrates closure of the tube body 1104 with the dispense cap 1102. FIG. 18D illustrates the filter tube assembly 1100 ready for compressions to filter a sample-containing fluid contained therein.

In preparing the filter tube assembly 1100 for use, a step may optionally include inserting the filter 1108 into the dispense cap 1102 as illustrated in FIG. 18A. Placement of the filter 1108 within the dispense cap 1102 may be in accordance with the present disclosure. In some other embodiments, the filter tube assembly 1100 may be supplied to a user with the filter 1108 already positioned in the dispense cap 1102.

In embodiments where the filter tube assembly 1100 includes a seal 1502, the user can remove the seal 1502. The user can remove the seal 1502 by, for example, gripping the tab portion 1506 and pulling upward as indicated by the dotted arrow in FIG. 18B. Once the seal 1502 is removed, sample can be added to the tube body 1104.

After sample is added to the tube body 1104, the tube body 1104 can be closed with the dispense cap 1102. The arm 1106 can bend and/or fold to allow the user to place the dispense cap 1102 on the tube body 1104 as indicated by the dotted arrow in FIG. 18C.

When the dispense cap 1102 is positioned on the tube body 1104, the user can compress the sides of the tube body 1104 as shown by the dotted arrows in FIG. 18D. The user may also invert the filter tube assembly 1100 to direct the filtered sample-containing fluid produced by the filter tube assembly 1100 into a receptable for receiving the filtered sample-containing fluid, for example a test device.

D. Filter Tube Assembly with Cup Filter and Separate Tube Body and Dispense Cap

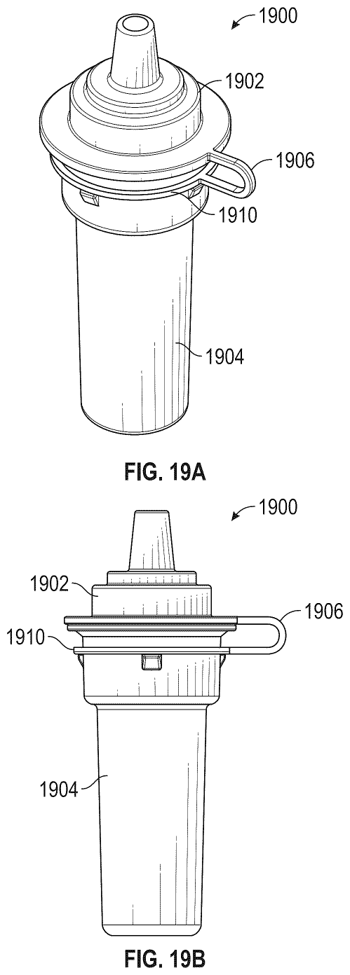

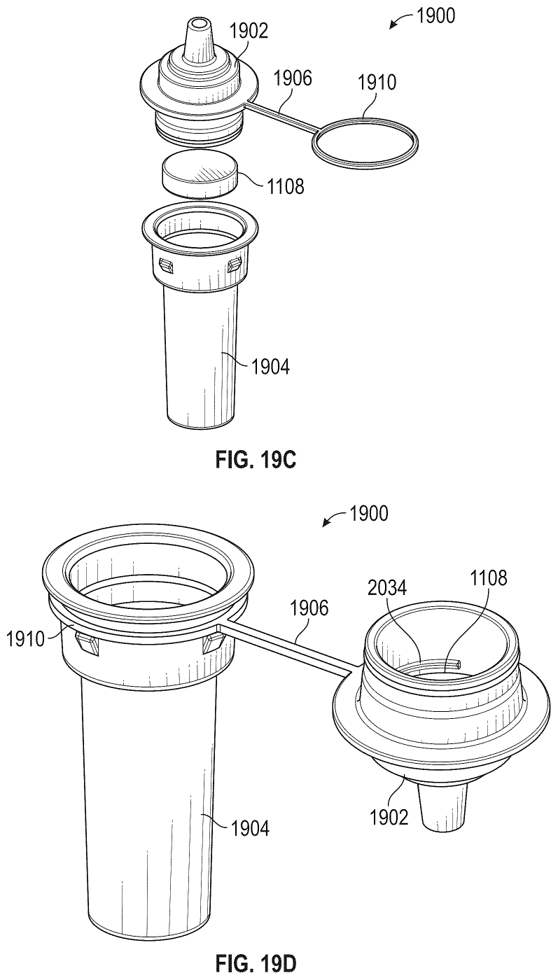

As previously discussed, embodiments of filter tube assemblies according to the present disclosure can include two-piece assemblies. For example, in some embodiments, a filter tube assembly may include a dispense cap and a tube body that are molded as separate components. FIGS. 19A-24 illustrate an example filter tube assembly 1900 that can be used for preparing a sample for a molecular assay according to an embodiment of the present disclosure, where the dispense cap 1902 and the tube body 1904 are separate components. FIG. 19A illustrates a perspective view of the filter tube assembly 1900. FIG. 19B illustrates a side view of the filter tube assembly 1900. FIG. 19C illustrates an exploded perspective view of the filter tube assembly 1900. FIG. 19D illustrates an angled view of the filter tube assembly 1900. The filter tube assembly 1900 can propel volumes of filtered sample-containing fluid in no more than one, two, three, or four compressions in accordance with the present disclosure (for example, with reference to filter tube assembly 1100). It will be understood that the filter tube assembly 1900 can include various features described herein in accordance with the present disclosure, including features described with reference to the filter tube assembly 1100. The dispense cap 1902 and tube body 1904 can be cast of different materials, as they are separate components.

As illustrated in FIGS. 19A-19D, the filter tube assembly 1900 may include a dispense cap 1902, a tube body 1904, and an arm 1906. The arm 1906 connects the dispense cap 1902 to a tether ring 1910. The tether ring 1910 can couple to the tube body 1904 such that the dispense cap 1902 and tube body 1904 are attached even while the tube body 1904 remains open. In other embodiments, the filter tube assembly 1900 may include two or more arms linking the dispense cap 1902 to the tether ring 1910. The arm 1906 may include a notch that can allow the arm 1906 to bend and/or fold.

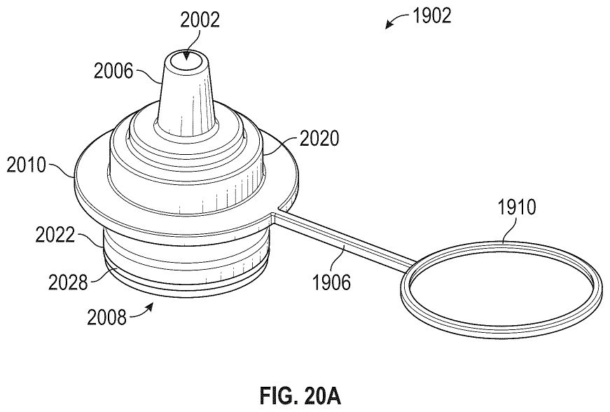

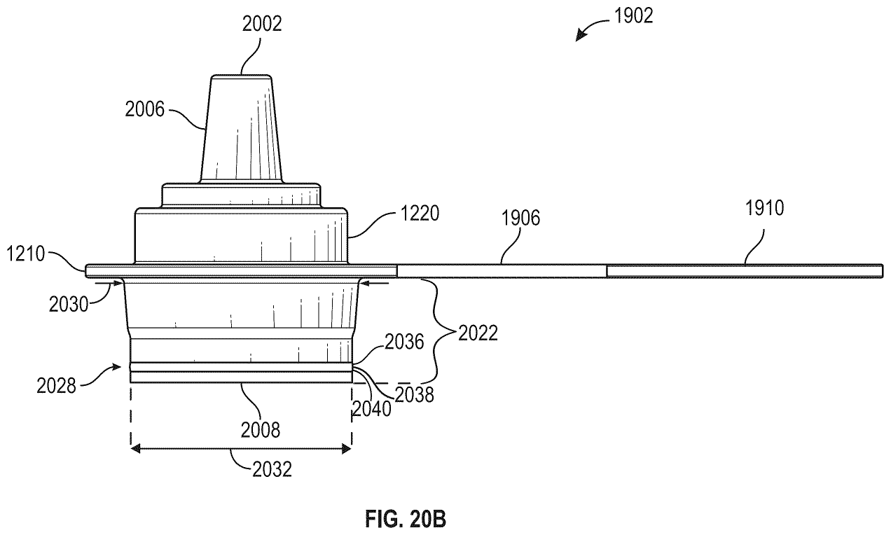

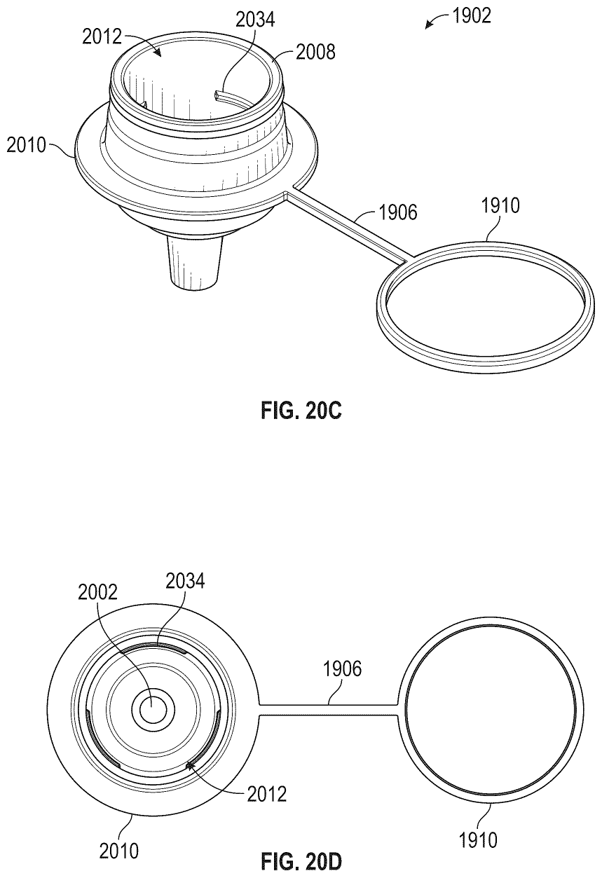

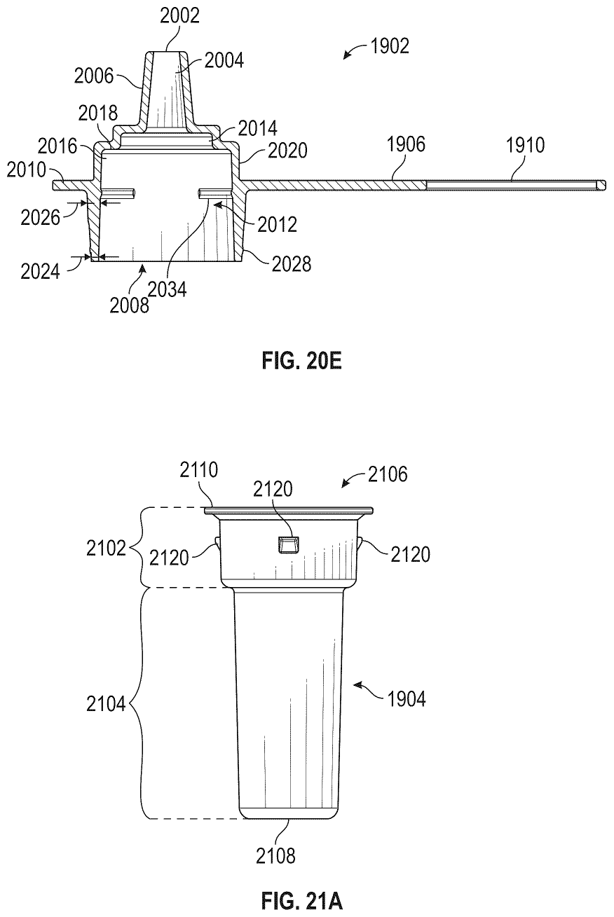

As illustrated in FIGS. 20A-20E, the dispense cap 1902 may include a collar 2022, an aperture 2002, a flow path 2004, a nozzle 2006, a proximal end 2008, a cap flange 2010, an interior space 2012, a transition zone 2014, a filter cavity 2016, a distal surface 2018, a cap body 2020, a ridge 2028, and retention bumps 2034. As discussed with reference to the collar 1222, the thickness of the collar 2022 may be non-uniform and may taper as the collar 2022 extends from the cap flange 2010. In other words, the proximal collar thickness 2024 may be less than the distal collar thickness 2026. Additionally or alternatively, as discussed with reference to the collar 1222, the diameter of the collar 2022 may decrease as the collar 2022 extends from the cap flange 2010 to the proximal end 2008 (that is, a distal collar diameter 2030 may be greater than a proximal collar diameter 2032). The diameters 2030 and 2032 may be sized in accordance with the present disclosure (for example, with reference to diameters 1230 and 1232, respectively). The dispense cap 1902 can engage a filter 1108 in accordance with the present disclosure (for example, as discussed with reference to the dispense cap 1102 and FIGS. 16A-16C). The flow path 2004 and/or transition zone 2014 may be sized and shaped in accordance with the present disclosure (for example, with reference to the flow path 1204 and the transition zone 1214). The filter 1108 (that is, the cup filter 1108) may be in accordance with embodiments of the present disclosure (for example, with reference to discussion regarding FIGS. 13A-13D). The retention bumps 2034 may be positioned proximate the filter cavity 2016. The retention bumps 2034 may prevent or inhibit the filter 1108 from moving away from the filter cavity 2016 in accordance with the present disclosure. The ridge 2028 is defined by an upper slope 2036, an apex 2038, and a lower slope 2040 in accordance with the present disclosure (for example, with reference to FIG. 12B).

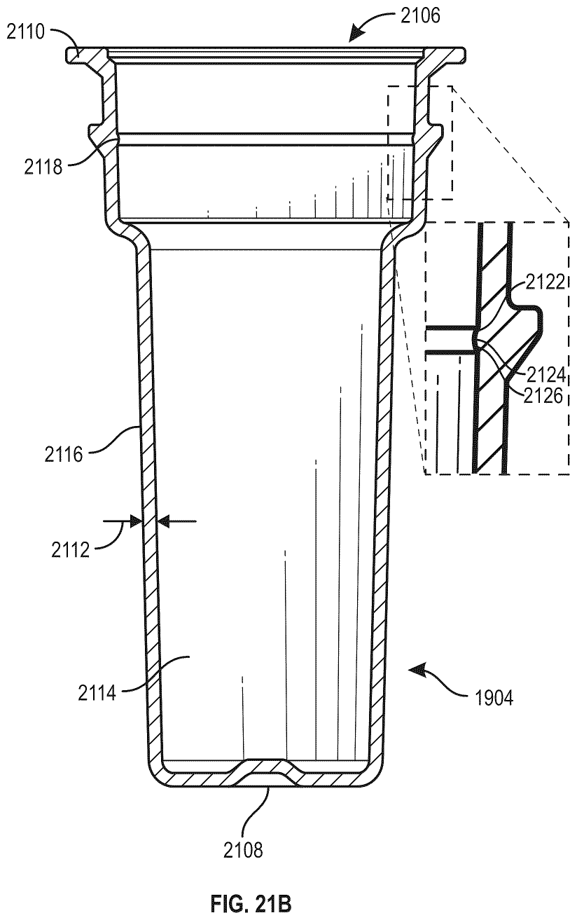

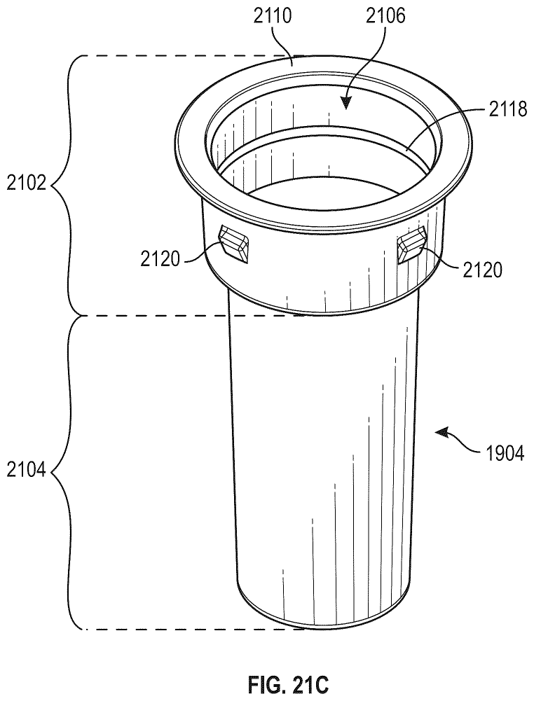

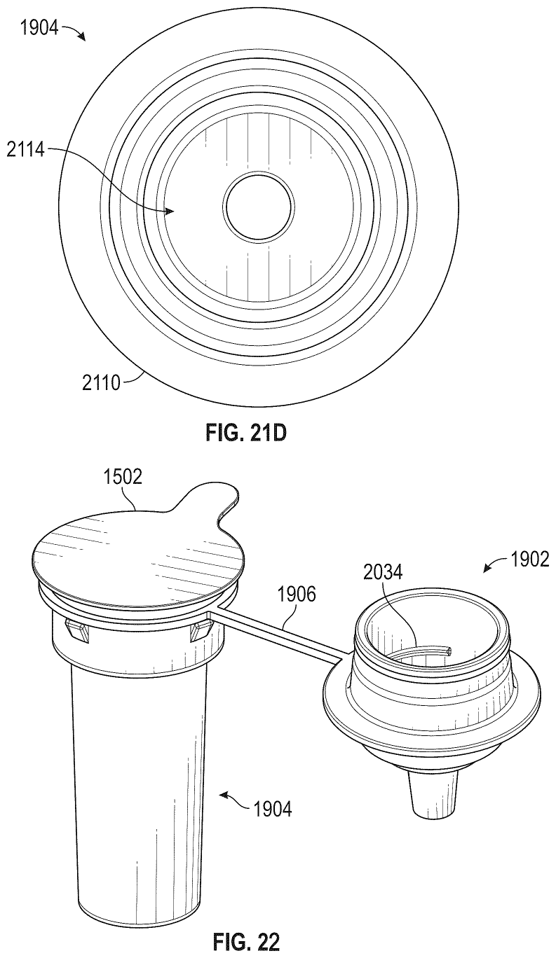

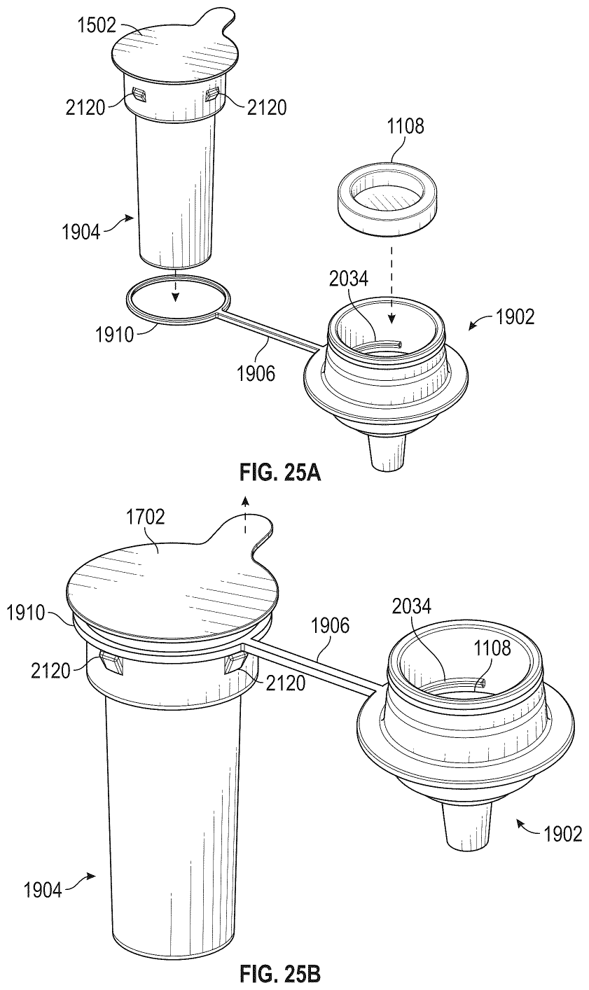

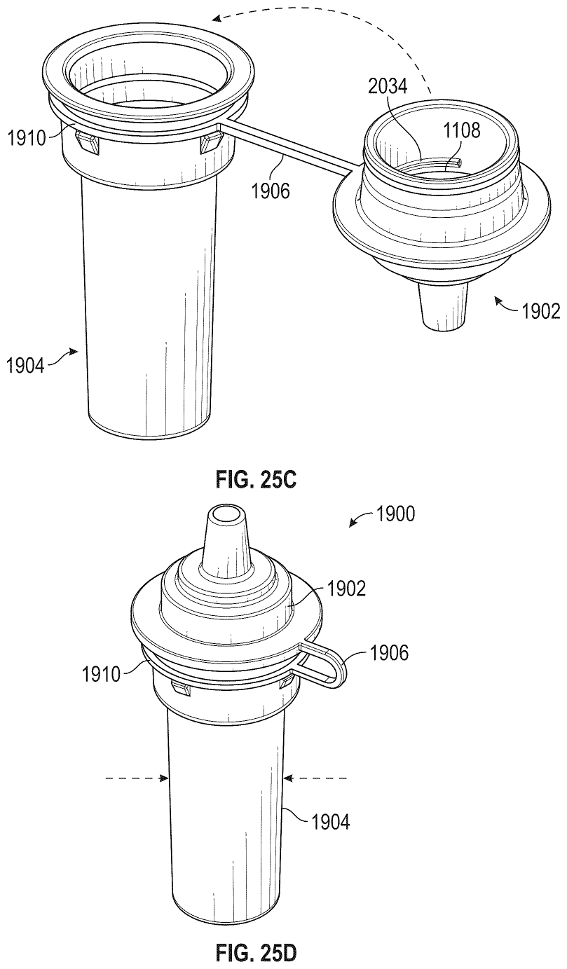

As illustrated in FIGS. 21A-21D, the tube body 1904 may include a tube flange 2110, an open end 2106, a closed end 2108, a tube wall 2116, an interior volume 2114, and teeth 2120. The tube body 1904 may optionally include a base region 2104 and a neck region 2102 that can include a ridge 2118. Each of the tube body 1904, the interior volume 2114, the neck region 2102, the tube wall 2116, the ridge 2118, the closed end 2108, and the open end 2106 may be sized, shaped, comprise materials, and/or include functionality in accordance with the present disclosure (for example, with reference to tube body 1104). The teeth 2120 may be positioned on an outer surface of the tube body 1904. The teeth 2120 may be positioned closer to the open end 2106 than the closed end 2108. In some examples, the teeth 2120 are positioned proximate the tube flange 2110. There may be two, three, four, or more teeth 2120 positioned along a circumference of the outer surface of the tube body 1904. The teeth 2120 may be positioned on the outer surface of the neck region 2102 of the tube body 1904. FIGS. 21A-21D illustrate an embodiment including four teeth 2120 positioned equidistant about a circumference of the outer surface of the tube body 1904. The teeth 2120 can engage the tether ring 1910 of the dispense cap 1102 (see FIGS. 25A-25B, which illustrate positioning of the tether ring 1910 of the dispense cap to the teeth 2120). The ridge 2118 is defined by an upper slope 2122, an apex 2124, and a lower slope 2126 in accordance with the present disclosure (for example, with reference to FIG. 14B).

FIG. 22 illustrates the filter tube assembly 1900 having a seal 1502. The seal 1502 may be in accordance with the present disclosure (for example, with reference to discussion regarding FIG. 15 and filter tube assembly 1100). As an illustrative example, the seal 1502 may be a foil heat-pressed seal. In another example, induction sealing is used to bond the seal 1502 to the tube flange 2110. Alternatively, in some embodiments, the filter tube assembly 1900 may include a travel cap that can create a fluid-tight barrier with the open end 2106 of the tube body 1904. Such a travel cap may be in accordance with the present disclosure (for example, with reference to discussion regarding travel cap 902).

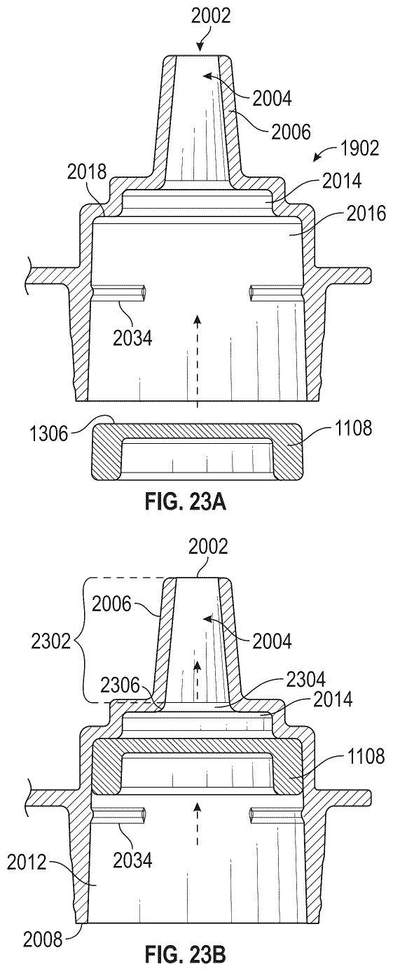

FIG. 23A shows placement of the filter 1108 within the dispense cap 1902. The dispense cap 1902 can include an aperture 2002, a flow path 2004, a nozzle 2006, a proximal end 2008, a transition zone 2014, a filter cavity 2016, a cap body 2020, a distal surface 2018, and the retention bumps 2034. The filter 1108 can be positioned within the dispense cap 1902 in accordance with the present disclosure (for example, with reference to FIGS. 16A and 16B and discussion of the filter 1108 positioned within the dispense cap 1102). FIG. 23B illustrates the filter 1108 positioned within the dispense cap 1902 and illustrates flow of liquid from the interior space 2012 through the filter 1108 to the flow path 2004. The aperture 2002, the flow path 2004, the nozzle 2006, the distal portion 2302, the curved portion 2306, the intermediate portion 2304, and the transition zone 2014 may be in accordance with embodiments of the present disclosure (for example, with reference to discussion of FIG. 16C and the dispense cap 1102).

In some embodiments, the interior volume 2114 of the tube body 1904 can be reduced in order to propel the sample-containing fluid from the filter tube assembly 1900 to a test device, for example for performing a molecular assay, such as but not limited to nucleic acid amplification. Reduction of the interior volume 2114 can be accomplished in several ways. In some embodiments, the material of the tube body 1904, in particular the base region 2104, is flexible enough to allow the user to compress the walls 2116 of the tube body 1904 to propel one or more streams of filtered sample-containing fluid to the test device through the aperture 2002 of the dispense cap 1902. The one or more streams can be continuous streams of fluid propelled through the aperture 2002. The flexibility can arise from a combination of thickness 2112 of the walls 2116 and modulus (for example, a Young's modulus) of the material included in the tube body 1904. For example, the thickness 2112 of the walls 2116 may be less than the thickness of portions of the dispense cap 1902. This combination of the thickness 2112 of walls 2116 and material of the tube body 1904 can be chosen so that the tube body 1904 can be compressed by a user. In another embodiment, the tube body 1904 can have thin sections in the walls 2116, running axially and/or radially, that give the tube body 1904 hinge points where the walls 2116 can flex while other portions of the walls 2116 are thicker and/or stiffer. The user can then compress the tube body 1904, which flexes at the thin hinge points thus reducing the interior volume 2114 and propelling the sample-containing fluid out through the filter 1108 and flow path 2004 and out the aperture 2002 of the dispense cap 1902 without the entire wall 2116 being thin enough to flex. Other configurations to propel a stream or other volume of sample-containing fluid from the tube body 1904 through the dispense cap 1902 are possible. In other embodiments, the tube body 1904 may not require compression or squeezing to dispense fluid from the container and may dispense drops of fluid upon inversion of the filter tube assembly 1900.

In some embodiments, the thickness of the material of the tube body 1904 at the tube flange 2110 may be equal to or larger than or equal to that of the thickness 2112 of the walls 2116. In some embodiments, the thickness 2112 of the material of the closed end 2108 may be equal to or larger than the thickness 2112 of the walls 2116. In some examples, the thickness 2112 of the walls 2116 may be about 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, or 1.25 mm or any value or range within or bounded by any of these ranges or values, although values outside these values or ranges can be used in some cases. Additionally or alternatively, in some examples the thickness 2112 is about 0.60 to 0.90 mm. Additionally or alternatively, in some examples the thickness 2112 is about 0.70 to 0.80 mm.

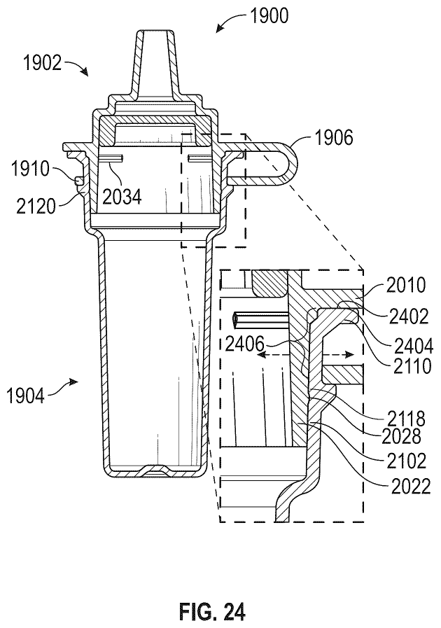

FIG. 24 illustrates the interaction of the dispense cap 1902 and the tube body 1904. The dispense cap 1902 may include a collar 2022 that can create a fluid-tight barrier against the neck region 2102 of the tube body 1904. Interaction of the collar 2022 and the neck region 2102 may be in accordance with embodiments of the present disclosure (for example, in accordance with filter tube assembly 1100 as discussed with respect to FIG. 17). The collar 2022 may contact an interior circumference of the open end 2106 of the tube body 1904, thereby creating a fluid-tight seal. When the dispense cap 1902 is attached to the open end 2106 of the tube body 1904, the collar 2022 and the walls of the neck region 2102 may press on each other as indicated by the dashed arrows in the inset of FIG. 24. When the dispense cap 1902 is attached to the open end 2106 of the tube body 1904, a proximal surface 2402 of the cap flange 2010 may abut a distal surface 2404 of the tube flange 2110. In some embodiments, the cap flange 2010 need not abut the tube flange 2110 to confirm an effective seal is formed between the dispense cap 1902 and the tube body 1904. In such embodiments, there may be sufficient interference fit to form an effective seal between the dispense cap 1902 and the tube body 1904 without abutting the cap flange 2010 and the tube flange 2110. Such an interference fit may occur at an assembly portion 2406. The interference fit at assembly portion 2406 may form a plug seal.

FIGS. 25A-25D illustrate a series of steps for preparing the filter tube assembly 1900 for use in filtering a sample-containing fluid. FIG. 25A illustrates insertion of the filter 1108 into the dispense cap 1902 and attachment of the dispense cap 1902 to the tube body 1904 via the tether ring 1910 and the arm 1906. FIG. 25B illustrates removal of the seal 1502 from the tube body 1904. FIG. 25C illustrates closure of the tube body 1904 with the dispense cap 1902. FIG. 25D illustrates the filter tube assembly 1900 ready for compressions to filter a sample-containing fluid contained therein.

In preparing the filter tube assembly 1100 for use, a step may optionally include inserting the filter 1108 into the dispense cap 1102 and/or attaching the dispense cap 1902 to the tube body 1904 via the arm 1906 and the tube body 1904, as illustrated in FIG. 25A. Placement of the filter 1108 in the dispense cap 1102 may be in accordance with the present disclosure. The tether ring 1910 can encompass a circumference of the tube body 1904 and engage with the teeth 2120. In some other embodiments, the filter tube assembly 1900 may be supplied to a user with the filter 1108 already positioned in the dispense cap 1102. In some embodiments, the filter tube assembly 1900 may be supplied to a user with the dispense cap 1902 already coupled to the tube body 1904 via the arm 1906 and the tether ring 1910.

In embodiments where the filter tube assembly 1900 includes a seal 1502, the user can remove the seal 1502. The user can remove the seal 1502 by, for example, gripping the tab portion 1506 and pulling upward as indicated by the dotted arrow in FIG. 25B. Once the seal 1502 is removed, sample can be added to the tube body 1904.

After sample is added to the tube body 1904, the tube body 1904 can be closed with the dispense cap 1902. The arm 1906 can bend and/or fold to allow the user to place the dispense cap 1902 on the tube body 1904 as indicated by the dotted arrow in FIG. 25C.

When the dispense cap 1902 is positioned on the tube body 1904, the user can compress the sides of the tube body 1904 as shown by the dotted arrows in FIG. 25D. The user may also invert the filter tube assembly 1900 to direct the filtered sample-containing fluid produced by the filter tube assembly 1900 into a receptable for receiving the filtered sample-containing fluid, for example a test device.

### E. Method of Preparing Sample Fluid

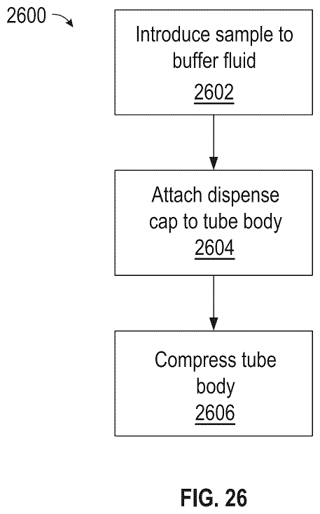

FIG. 26 illustrates an example method 2600 of using a filter tube assembly to prepare a sample for a molecular assay in accordance with the present disclosure.

At step 2602, a sample is introduced to a buffer, thereby creating a sample-containing fluid. In some embodiments, the buffer may be located within a tube body of the filter tube assembly when the sample is introduced to the buffer. In some embodiments, the sample may be introduced to the buffer at a location other than the tube body of the filter tube assembly. In such embodiments, a user can dispense the sample-containing fluid to the tube body of the filter after the sample has been introduced to the buffer.

At step 2604, the dispense cap is attached to the tube body. In some embodiments, attaching the dispense cap to the tube body includes engaging snap-fit features (for example, ridges and/or indentations) of the dispense cap and the tube body. In such embodiments, the snap-fit features, for example ridges and/or indentations, may provide a user with tactile or auditory feedback that the dispense cap is secured to the tube body. In such embodiments, engaging the snap-fit features may create a fluid-tight seal between the dispense cap and the tube body. In some embodiments, attaching the dispense cap to the tube body includes engaging a collar of the dispense cap with an open end of the tube body, thereby creating a fluid-tight seal (for example, a plug seal).

At step 2606, the tube body is compressed. Compressing the tube body may propel the sample-containing fluid held within the tube body through a filter positioned in the dispense cap. After passing through the filter, the sample-containing fluid may be propelled out of the dispense cap. In some embodiments, the user may compress the tube body no more than 1, 2, 3, or 4 times to propel the desired volume of filtered sample-containing fluid from the filter tube assembly. In some embodiments, the filtered sample-containing fluid is propelled from the filter tube assembly to a test device, for example a test device where a molecular assay is performed.

### EXAMPLES

### Example 1

### Filter Efficacy

Testing was performed to determine assay sensitivity of filtered sample-containing fluid. Filter tube assemblies in accordance with the embodiment of FIGS. 1A-7 of the present disclosure were used, each filter tube assembly including one of several different types of filter material as a filter 102. Table 1 lists reactivity for various filters, including HDC40, HDC5, and HDC20 (LA1244) (melt blown polypropylene filters by Pall Corporation (Port Washington, NY)); an Omnipore® filter (a hydrophobic PTFE filter by MilliporeSigma (Burlington, Massachusetts (hereinafter “Omnipore”)); Poly45 μM (a hydrophobic polypropylene filter by MilliporeSigma); and Vivid ACG (a hydrophilic glass fiber filter by Pall Corporation, provided in the BD Veritor™ At-Home COVID-19 Test, the FluA+B Rapid Antigen Test, and the RSV Rapid Antigen Test (Becton Dickinson, Franklin Lakes, New Jersey) (hereinafter “Veritor filter”)). The filters were used to prepare sample-containing fluid for assays measuring presence of Chlamydia trachomatis (CT), Neisseria gonorrhoeae (GC), and an internal control (IC). Reactivity for an unfiltered sample is also listed. Time to detection (Td) is a measure of amplification time, in minutes. Higher Td may be due to inhibition of the assay by inhibitors. Thus, a lower Td may indicate that the filter under test removed a larger portion of the inhibitors from the sample-containing fluid compared to an unfiltered sample-containing fluid.

TABLE 1
Reactivity and Td

| Assay Type | Filter Type | Average Reactivity | Avg. reactive Td |
|---|---|---|---|
| CT | HDC40 | 90% | 3.78 |
| | HDC5 | 90% | 4.06 |
| | HDC20 | 90% | 3.85 |
| | Omnipore | 60% | 4.22 |
| | Poly45 μM | 60% | 4.37 |
| | Veritor Filter | 90% | 3.97 |
| | Unfiltered | 50% | 3.93 |
| GC | HDC40 | 90% | 3.20 |
| | HDC5 | 100% | 3.09 |
| | HDC20 | 100% | 3.17 |
| | Omnipore | 70% | 3.00 |
| | Poly45 μM | 70% | 3.39 |
| | Veritor Filter | 100% | 3.16 |
| | Unfiltered | 50% | 3.45 |
| IC | HDC40 | 100% | 3.23 |
| | HDC5 | 100% | 3.22 |
| | HDC20 | 100% | 3.08 |
| | Omnipore | 80% | 3.13 |
| | Poly45 μM | 80% | 3.61 |
| | Veritor Filter | 100% | 3.13 |
| | Unfiltered | 80% | 4.07 |

All of the filter material tested had a higher reactivity than the unfiltered sample, for each of the three assay types. Accordingly, embodiments of a filter tube assembly according to the present disclosure demonstrated improved assay reactivity and sensitivity relative to an unfiltered sample-containing fluid.

Turbidity of the filtered sample-containing fluid produced by each filter type was measured at a wavelength of 600 nm. Table 2 shows mean OD 600 for filtered sample-containing fluid of each of the filter types, wherein OD 600 refers to the optical density of the sample-containing fluid measured at the 600 nm wavelength. Of the filter types, HDC40, HDC5, HDC20, and the Veritor Filter produced filtered sample-containing fluid that had significantly different OD600 than the OD600 of unfiltered sample-containing fluid at p=0.05.

TABLE 2
Turbidity at OD600

| Filter Type | Mean OD600 |
|---|---|
| HDC40 | 0.038 |
| HDC5 | 0.057 |
| HDC20 | 0.046 |
| Omnipore | 0.192 |
| Poly45 μM | 0.309 |
| Veritor Filter | 0.042 |
| Unfiltered | 0.415 |

Without being bound to a particular theory, it is believed that a lower OD600 indicates that more potential inhibitors are removed by a filter of the filter tube assembly according to the present disclosure. Turbidity was shown to be correlated with assay sensitivity for both CT and GC assays at p=0.05.

### Example 2

### Efficacy of Sintered Polyethylene Filters

Testing was performed to determine the effect on assay sensitivity of several different types of filter material. Table 3 lists reactivity for the following sintered polyethylene filters by Porex Corporation (Richmond, Virginia): Porex 120, Porex 266, Porex 269, and Porex 595. The Veritor Filter, a hydrophilic glass fiber filter, was also tested. The filters were used with filter tube assemblies in accordance with the embodiment of FIGS. 1A-7 of the present disclosure to perform assays measuring presence of CT, GC, and an internal control (IC). Reactivity for unfiltered sample is also listed.

TABLE 3
Reactivity and Td

| Assay Type | Filter Type | Average Reactivity | Avg. reactive Td |
|---|---|---|---|
| CT | Porex 120 | 100% | 3.80 |
| | Porex 266 | 80% | 3.67 |
| | Porex 269 | 80% | 3.42 |
| | Porex 595 | 100% | 3.89 |
| | Veritor Filter | 100% | 3.55 |
| GC | Porex 120 | 90% | 3.20 |
| | Porex 266 | 90% | 3.24 |
| | Porex 269 | 100% | 3.18 |
| | Porex 595 | 100% | 3.24 |
| | Veritor Filter | 80% | 3.27 |
| IC | Porex 120 | 100% | 3.09 |
| | Porex 266 | 90% | 3.05 |
| | Porex 269 | 100% | 3.02 |
| | Porex 595 | 100% | 3.05 |
| | Veritor Filter | 100% | 3.15 |

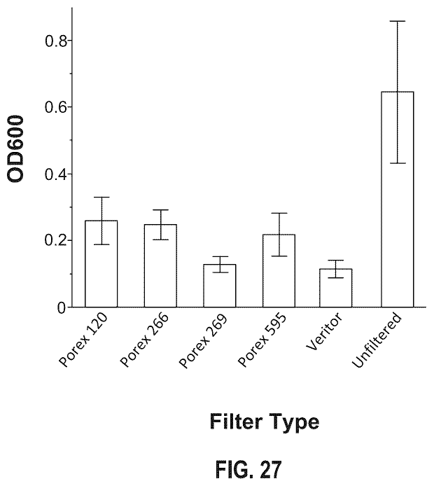

FIG. 27 plots optical density at a wavelength of 600 nm (OD600) of sample-containing fluid filtered by each of the five filters tested using filter tube assemblies according to the present disclosure, along with the OD600 of unfiltered sample-containing fluid. Each of the filters reduced OD600 relative to the unfiltered sample-containing fluid.

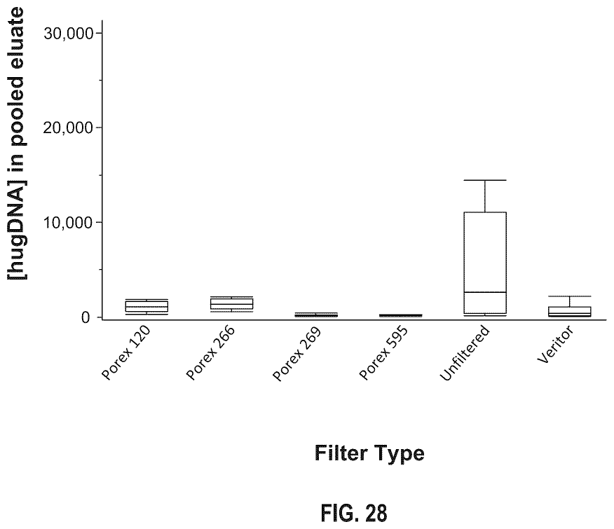

FIG. 28 is a box plot of the concentration of human genomic DNA (hugDNA) of sample-containing fluid filtered by each of the five filters tested using filter tube assemblies according to the present disclosure, along with the hugDNA of unfiltered sample-containing fluid, measured by qPCR. FIG. 28 shows that each filter type tested reduced hugDNA compared to unfiltered sample-containing fluid.

### Example 3

### Filtering Urine Samples

Three different urine samples that were found to be inhibitory to CT, GC, and/or IC archaeal polymerase amplification (APA) assays were tested with and without filtration. Using filter tube assemblies in accordance with the present disclosure having a Porex 120 filter, these urine samples were filtered before being subjected to the assays.

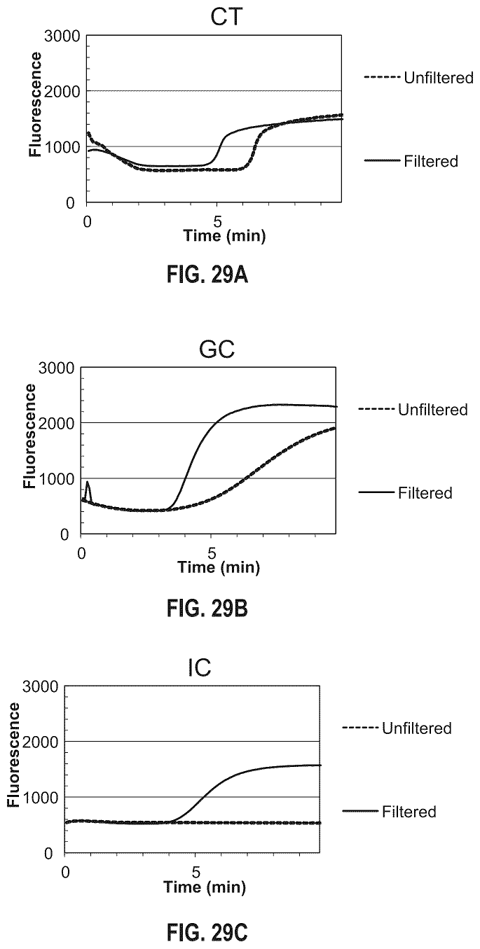

FIGS. 29A, 29B, and 29C plot fluorescence of the CT, GC, and IC assays respectively. The solid lines indicate the fluorescence of the filtered samples, whereas the dotted lines indicate fluorescence of the unfiltered samples. For all three assays, filtration improved amplification. For the IC assay shown in FIG. 29C, amplification was rescued. In particular, the unfiltered sample used in the internal control assay did not amplify, whereas the increase in fluorescence of the filtered sample indicates that the filtered sample successfully amplified.

### Example 4

### Filtration of Samples for Trichomonas Vaginalis Assay

Trichomonas vaginalis (TV) is an infection-causing parasite. The TV trophozoites are 7-30 μm long, a range of lengths that is about 20-100 times larger than many types of bacteria. TV may be detectable, if present, in a vaginal swab specimen. When filtering a sample in preparation for a molecular assay, for example filtering using a filter tube assembly in accordance with the present disclosure, it may be desirable that the filter does not remove the TV trophozoites (thereby comprising or reducing assay sensitivity).

Samples containing a known amount of TV were filtered in accordance with embodiments of the present disclosure. In particular, the BDMAX™ CTGCTV2 assay (Becton Dickinson, Franklin Lakes) was used as a model for measuring relative cycle threshold (Ct) of TV samples, before and after filtration using a filter tube assembly according to the present disclosure, with GC as a control. A filter tube assembly in accordance with the present disclosure, including a Porex 120 filter, was used to filter 1 mL of BDMAX™ buffer having a TV concentration of 1×10⁵ trophozoites/mL. Another 1 mL of BDMAX™ buffer having a TV concentration of 1×10⁵ trophozoites/mL was tested in parallel as an “unfiltered” sample. A filter tube assembly in accordance with the present disclosure, including a Porex 120 filter, was used to filter 1 mL of BDMAX™ buffer having a GC concentration of 1×10⁴ cfu/mL GC.

Another 1 mL of BDMAX™ buffer having a GC concentration of 1×10⁴ cfu/mL GC was tested in parallel as an “unfiltered” sample.

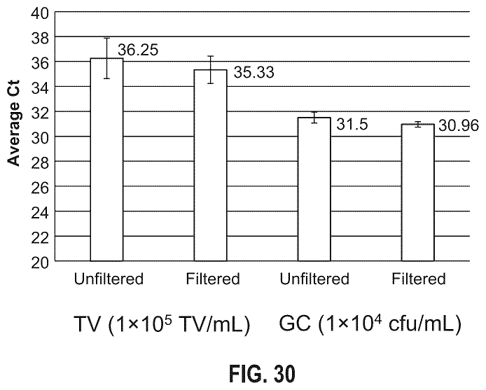

The filtered and unfiltered TV buffer and GC buffer solutions were subjected to an amplification assay. FIG. 30 plots the average Ct score for these samples, with the error bars indicating standard deviation. There was no significant difference in the Ct of the unfiltered and filtered TV samples. There was no significant difference in the unfiltered and filtered GC samples. These results suggest that relatively large parasites, for example TV, are not removed during filtering of vaginal samples using filter tube assemblies according to the present disclosure.

### Terminology

Unless defined otherwise, all technical and scientific terms used herein have the same meaning as is commonly understood by one of ordinary skill in the art.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least-limiting meaning, which includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Conditional language used herein, such as, among others, “can,” “could,” “might,” “may,” and the like, unless specifically stated otherwise, or otherwise understood within the context as used, is generally intended to convey that certain features, elements and/or steps are optional. Thus, such conditional language is not generally intended to imply that features, elements and/or steps are in any way required. The terms “comprising,” “including,” “having,” and the like are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Any methods disclosed herein need not be performed in the order recited. The methods disclosed herein include certain actions taken by a practitioner; however, they can also include any third-party instruction of those actions, either expressly or by implication.

While the above detailed description has shown, described, and pointed out novel features, it can be understood that various omissions, substitutions, and changes in the form and details of the devices, systems, and methods can be made without departing from the spirit of the present disclosure. For instance, features as discussed within the various example embodiments can be interchanged to the extent they are compatible. As can be recognized, certain portions of the description herein can be embodied within a form that does not provide all of the features and benefits set forth herein, as some features can be used or practiced separately from others. Consequently, it is not intended that the present disclosure be limited to the specific embodiments disclosed herein, but that it covers all modifications and alternatives coming within the true scope and spirit of the present disclosure.

1. A filter tube assembly for preparing a sample-containing fluid for use with a molecular assay, the assembly comprising:
a dispense cap comprising a flow path and an aperture,
a filter positioned within the dispense cap; and
a tube body comprising an open end and a closed end, the tube body configured to hold a sample-containing fluid, the dispense cap configured to secure to the open end of the tube body, at least a portion of the tube body comprising a flexible material configured to be compressed to force at least a portion of the sample-containing fluid through the filter, the flow path, and the aperture when the dispense cap is secured to the open end of the tube body and the flexible material is compressed, the dispense cap configured to propel about 2.5 mL to about 3.0 mL of filtered sample-containing fluid in continuous streams through the aperture during no more than two compressions of the flexible material.

2. The filter tube assembly of claim 1, wherein the filter tube assembly is configured to propel a continuous stream of at least about 1 mL of filtered sample-containing fluid through the aperture upon a single compression of the tube.

3. The filter tube assembly of claim 1, wherein the flow path comprises a transition zone positioned proximate the filter, the transition zone configured to reduce back pressure on the filter upon compression of the flexible material, and wherein a diameter of the filter is from about 1.2 to about 1.6 times larger than a diameter of the transition zone.

4. (canceled)

5. The filter tube assembly of claim 4, wherein the diameter of the filter ranges from about 12 mm to about 16 mm and wherein the diameter of the transition zone is between about 8 mm to about 12 mm.

6. (canceled)

7. (canceled)

8. The filter tube assembly of claim 1, wherein the filter is a cup filter comprising walls configured to secure the filter to the dispense cap.

9. (canceled)

10. (canceled)

11. (canceled)

12. (canceled)

13. The filter tube assembly of claim 1, wherein the dispense cap comprises a high-density polyethylene (HDPE) or a polypropylene, and wherein the tube body material comprises a linear low-density polyethylene (LLDPE).

14. (canceled)

15. (canceled)

16. (canceled)

17. (canceled)

18. (canceled)

19. (canceled)

20. The filter tube assembly of claim 1, wherein a thickness of at least a portion of walls of the tube body is between about 0.2 mm and about 1.0 mm.

21. The filter tube assembly of claim 1, wherein the filter comprises a plurality of openings having diameter between about 1 μm and about 250 μm, and wherein the filter comprises a polyethylene, a glass fiber, a polypropylene, or a polytetrafluoroethylene.

22. (canceled)

23. (canceled)

24. (canceled)

25. (canceled)

26. The filter tube assembly of claim 1, wherein the aperture comprises a diameter, and wherein the diameter is between about 2.8 mm and about 3.2 mm.

27. The filter tube assembly of claim 1, wherein the filter is configured to filter molecules entering through a first surface of the filter and exiting through an opposing second surface of the filter, and wherein the surface area of the first surface of the filter is from about 100 mm² to about 200 mm².

28. The filter tube assembly of claim 27, wherein a ratio a:v of the surface area of the first surface of the filter to the internal volume of the tube body is between about 20 m 1 to about 40 m⁻¹.

29. The filter tube assembly of claim 1, wherein the tube body further comprises a snap fit ridge and a flange, the snap fit ridge configured to matingly engage with an indentation of the dispense cap, the flange configured to contact a proximal end of the dispense cap.

30. (canceled)

31. (canceled)

32. The filter tube assembly of claim 1, wherein the tube body and dispense cap are cast as a single piece of plastic, and wherein the filter tube assembly comprises at least one tether connecting the tube body to the dispense cap.

33. The filter tube assembly of claim 32, wherein the plastic of the tube body and dispense cap has a Young's modulus between 200 and 700 MPa.

34. The filter tube assembly of claim 1, comprising a seal covering the open end of the tube body.

35. (canceled)

36. The filter tube assembly of claim 1, further comprising a buffer contained within an interior volume of the tube body.

37. The filter tube assembly of claim 1, wherein the filter is configured to prepare the sample-containing fluid for use in a gonorrhea assay, a chlamydia assay, or a trichomoniasis assay.

38. (canceled)

39. The filter tube assembly of claim 1, wherein the dispense cap further comprises a collar, and the tube body comprises a neck region configured to engage the collar of the dispense cap to form a fluid-tight seal, wherein the collar and neck region are configured to engage via an interference fit.

40. The filter tube assembly of claim 39, wherein a thickness of the wall at a proximal end of the dispense cap collar is less than a thickness of the wall at a distal end of the dispense cap collar.

41. The filter tube assembly of claim 39, wherein an outer diameter of the wall at a proximal end of the dispense cap collar is less than an outer diameter of the wall at a distal end of the dispense cap collar.

42. The filter tube assembly of claim 39, wherein the dispense cap collar comprises a first ridge and the neck region of the tube body comprises a second ridge, the first ridge and the second ridge configured to slidingly engage.

43. The filter tube assembly of claim 39, wherein the tube body further comprises a base portion positioned proximate the closed end of the tube body, the base portion comprising a diameter that is less than a diameter of the neck region.

44. The filter tube assembly of claim 39, wherein the dispense cap comprises one or more retention bumps configured to retain the filter within the dispense cap.

45. A method of preparing a sample for a molecular assay, comprising:
securing a dispense cap to an open end of a tube body, the dispense cap comprising a filter;
compressing the tube body no more than two times, thereby propelling a sample-containing buffer from an interior volume of the tube body through the filter and out the dispense cap in continuous streams, while retaining in the filter at least some inhibitors of a molecular assay from the sample-containing buffer, wherein compressing the tube body propels at least from about 2.5 mL to about 3.0 mL of filtered sample-containing buffer from the dispense cap; and
performing the molecular assay using the filtered sample-containing buffer.

46. (canceled)

47. (canceled)

48. The method of any of claim 45, wherein performing the molecular assay comprises amplification of nucleic acids in the filtered sample-containing buffer.

49. (canceled)

50. (canceled)

51. (canceled)

52. (canceled)

53. The method of claim 45, wherein the dispense cap comprises a flow path and an aperture, the flow path comprises a transition zone, and a diameter of the filter is about 1.2 to about 1.6 times larger than a diameter of the transition zone of the flow path.

54. The method of claim 45, wherein the diameter of the filter ranges from about 12 mm to about 16 mm and the diameter of the transition zone ranges from about 8 mm to about 12 mm.

55. The method of claim 45, wherein compressing the tube body comprises filtering molecules from the sample-containing buffer that enters a first surface of the filter and exits through a second surface of the filter, the surface area of the filter being between about 100 mm² and about 200 mm².

56. The method of claim 45, wherein a ratio a:v of the surface area of the first surface of the filter to the internal volume of the tube body is between about 20 m⁻¹ to about 40 m⁻¹.

57. (canceled)

58. (canceled)

59. (canceled)

60. (canceled)
